(19) United States
(12) Patent Application Publication
(10) Pub. No.: US 2024/0141427 A1
(43) Pub. Date: May 2, 2024

(54) POLYMERASES, COMPOSITIONS, AND METHODS OF USE

(71) Applicant: ILLUMINA, INC., San Diego, CA (US)

(72) Inventors: Misha Golynskiy, San Diego, CA (US); Rahman Rahman Pour, San Diego, CA (US); Jiawen Li, San Diego, CA (US); Ryan Craig, San Diego, CA (US); Hamed Tabatabaei Ghomi, Cambridge (GB); Saurabh Nirantar, Singapore (SG); Hsu Myat Noe, Singapore (SG); Lin Hui Chang, Singapore (SG); Yvonne Devadas, Singapore (SG); Jing Wen Lim, Singapore (SG); Kay Klausing, San Diego, CA (US); Humberto Rojo, San Diego, CA (US); Eric Murtfeldt, San Diego, CA (US); Chris Garcia, San Diego, CA (US)

(73) Assignee: ILLUMINA, INC., San Diego, CA (US)

(21) Appl. No.: 18/373,620

(22) Filed: Sep. 27, 2023

Related U.S. Application Data
(60) Provisional application No. 63/412,241, filed on Sep. 30, 2022.
(60) Provisional application No. 63/433,971, filed on Dec. 20, 2022.

(51) Int. Cl. C12Q 1/6874; C12N 9/12

(52) U.S. Cl. CPC C12Q 1/6874; C12N 9/1252; C12Y 207/07007

(57) ABSTRACT

Presented herein are altered polymerase enzymes for improved incorporation of nucleotides and nucleotide analogues, in particular altered polymerases that maintain low error rate, low phasing rate, or increased incorporation rate for a second generation ffN under reduced incorporation times, as well as methods and kits using the same.

This application claims the benefit of U.S. Provisional Application Ser. No. 63/412,241, filed Sep. 30, 2022, and 63/433,971, filed Dec. 20, 2022, each of which is incorporated by reference herein in its entirety.

### SEQUENCE LISTING

This application contains a Sequence Listing electronically submitted via EFS-Web to the United States Patent and Trademark Office as an XML file entitled “0531.00238US01.xml” having a size of 53,163 bytes and created on Sep. 5, 2023. The information contained in the Sequence Listing is incorporated by reference herein.

### FIELD

The present disclosure relates to, among other things, altered polymerases for use in performing a nucleotide incorporation reaction, particularly in the context of nucleic acid sequencing by synthesis.

### BACKGROUND

Sequencing by synthesis (SBS) technology relies on DNA polymerases and modified nucleotides as components of the sequencing process. The modified nucleotides typically include a 3′-hydroxy protecting group (also referred to as a blocking group) to prevent the polymerase from incorporating additional nucleotides into the polynucleotide chain after the base on the nucleotide is added by the DNA polymerase. After the identity of an added base is determined, the 3′-hydroxy protecting group is removed, and the next modified nucleotide is added by the polymerase. Useful protecting groups prevent additional nucleotides from being added to the polynucleotide chain and are reversible, e.g., are easily removable from the sugar moiety of the nucleotide without causing damage to the polynucleotide chain. Furthermore, the modified nucleotide needs to be compatible with the polymerase used to incorporate it into the polynucleotide chain.

### SUMMARY OF THE APPLICATION

The present disclosure provides polymerases that have improved incorporation rates of modified nucleotides described in, for instance, U.S. Pat. No. 11,293,061. These polymerases exhibit improved sequencing performance over the enzymes currently employed in SBS when tested under fast cycle time SBS conditions with the second generation fully functional nucleotides described herein.

Provided herein are altered archaeal Family B DNA polymerases. In one embodiment, an altered archaeal Family B DNA polymerase includes an amino acid substitution mutation at a position functionally equivalent to an amino acid in a reference archaeal Family B DNA polymerase of SEQ ID NO:1, and the altered archaeal Family B DNA polymerase is capable of incorporating a modified nucleotide including a 3′-OH acetal blocking group or a 3′-OH thiocarbamate blocking group at (i) a lower error rate, (ii) a lower phasing rate, or both (i) and (ii), compared to SEQ ID NO:1; and an amino acid substitution mutation at: position Arg58 to Leu; position Tyr261 to Gly; position Asn269 to Gly or Val; position Phe283 to Ile or Lys; position Pro328 to Asp; position Met329 to Thr; position Gln332 to Ser; position Leu333 to His or Ile; position Ser347 to Asp or Arg or Glu or Val or Thr; position Asn399 to Met or Ala; position Phe405 to Met or Lys or Gln; position Arg406 to Met; position Ile410 to Val; position Ile412 to Val; position Glu458 to Gly; position Glu459 to Thr or Asp; position Gln461 to Trp; position Ala469 to Glu; position Tyr481 to Ile; position Va1485 to Asn or Gly or Met or Gln or Ser or Thr; position Ile486 to Leu; position Trp504 to Asn; position Lys507 to His or Pro; position Glu511 to Lys or Met or Arg; position Trp516 to Ile or Lys or Met or Gln or Leu; position Tyr520 to Ala; position Ile521 to Thr; position Met523 to Ile or Thr; position Arg526 to Asn; position Glu527 to Ile; position Leu528 to Thr; position Ile567 to Leu; position Asn568 to Gln; position Leu571 to Phe or Ile or Met or Trp; position Glu576 to Leu; position Glu580 to Ile or Lys or Gln or Arg or Val or Met; position Thr590 to Lys; position Ala595 to Met; or position Ile603 to Asp. The present disclosure also provides altered archaeal Family B DNA polymerases that include at least two amino acid substitution mutations, at least three amino acid substitution mutation, at least four amino acid substitution mutations, or at least five amino acid substitution mutations.

Also provided herein are altered Family B DNA polymerases. In one embodiment, an altered Family B DNA polymerase includes an amino acid sequence that is at least 80% identical to amino acid sequence SEQ ID NO:1 and one or more substitution mutations as described herein. In another embodiment, altered Family B DNA polymerase is an altered archaeal Family B DNA polymerase that include one or more substitution mutations as described herein. The one or more substitution mutations include, but are not limited to, an amino acid substitution mutation at a position functionally equivalent to Glu580 in a reference archaeal Family B DNA polymerase of SEQ ID NO:1, an amino acid substitution mutation at a position functionally equivalent to Phe405 and Va1485 in a reference archaeal Family B DNA polymerase of SEQ ID NO:1, an amino acid substitution mutation at a position functionally equivalent to Phe405 and Ile410 in a reference archaeal Family B DNA polymerase of SEQ ID NO:1, an amino acid substitution mutation at a position functionally equivalent to Phe140 and Ser407 in a reference archaeal Family B DNA polymerase of SEQ ID NO:1, an amino acid substitution mutation at a position functionally equivalent to Leu403, Ala408, Ile410, and Gly497 in a reference archaeal Family B DNA polymerase of SEQ ID NO:1, an amino acid substitution mutation at a position functionally equivalent to Phe405, Ile410, Ile412, Thr514, and Ile521 in a reference archaeal Family B DNA polymerase of SEQ ID NO:1, or an amino acid substitution mutation at a position functionally equivalent to Phe405, Ala408, Ile410, Ile412, Thr514, Ile521 in a reference archaeal Family B DNA polymerase of SEQ ID NO:1, where the altered archaeal Family B DNA polymerase is capable of incorporating a modified nucleotide including a 3′-OH acetal blocking group or a 3′-OH thiocarbamate blocking group at (i) a lower error rate, (ii) a lower phasing rate, or both (i) and (ii), compared to SEQ ID NO:1. In one embodiment,

Terms used herein will be understood to take on their ordinary meaning in the relevant art unless specified otherwise. Several terms used herein and their meanings are set forth below.

The term “and/or” means one or all of the listed elements or a combination of any two or more of the listed elements.

The words “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

The terms “comprises” and variations thereof do not have a limiting meaning where these terms appear in the description and claims.

It is understood that wherever embodiments are described herein with the language “include,” “includes,” or “including,” and the like, otherwise analogous embodiments described in terms of “consisting of” and/or “consisting essentially of” are also provided.

Unless otherwise specified, “a,” “an,” “the,” and “at least one” are used interchangeably and mean one or more than one.

Conditions that are “suitable” for an event to occur or “suitable” conditions are conditions that do not prevent such events from occurring. Thus, these conditions permit, enhance, facilitate, and/or are conducive to the event.

As used herein, “providing” in the context of a polymerase or a composition means making the polymerase or composition, purchasing the polymerase or composition, or otherwise obtaining the polymerase or composition.

Also herein, the recitations of numerical ranges by endpoints include all numbers subsumed within that range (e.g., 1 to 5 includes 1, 1.5, 2, 2.75, 3, 3.80, 4, 5, etc.).

Reference throughout this specification to “one embodiment,” “an embodiment,” “certain embodiments,” or “some embodiments,” etc., means that a particular feature, configuration, composition, or characteristic described in connection with the embodiment is included in at least one embodiment of the disclosure. Thus, the appearances of such phrases in various places throughout this specification are not necessarily referring to the same embodiment of the disclosure. Furthermore, the particular features, configurations, compositions, or characteristics may be combined in any suitable manner in one or more embodiments.

The above summary of the present disclosure is not intended to describe each disclosed embodiment or every implementation of the present disclosure. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

The following detailed description of illustrative embodiments of the present disclosure may be best understood when read in conjunction with the following drawings.

FIGS. 1A to 1E is a schematic showing alignment of polymerase amino acid sequences from Thermococcus sp. 9° N-7 (9° N, SEQ ID NO:9), Thermococcus litoralis (Vent, SEQ ID NO:10 and Deep Vent, SEQ ID NO:11), Thermococcus waiotapuensis (Twa, SEQ ID NO:12), Thermococcus kodakarensis (KOD, SEQ ID NO:13), Pyrococcus furiosus (Pfu, SEQ ID NO:14), Pyrococcus abyssi (Pab, SEQ ID NO:15). An “*” (asterisk) indicates positions which have a single, fully conserved residue between all polymerases. A “:” (colon) indicates conservation between groups of strongly similar properties as below -roughly equivalent to scoring >0.5 in the Gonnet PAM 250 matrix. A “.” (period) indicates conservation between groups of weakly similar properties as below - roughly equivalent to scoring =<0.5 and >0 in the Gonnet PAM 250 matrix.

FIGS. 2A-2B shows some exemplary mutations considered for secondary screening.

FIG. 3 shows Tier 1 mutant performance resulting from secondary screening.

FIGS. 4A-4B shows Tier 2 mutant performance resulting from secondary screening.

FIG. 5 shows examples of double mutant performance during secondary screening.

FIG. 6 shows examples of polymerases (polymerases A, B, E, F, H, I, J, L, N, and 0) tested in 150 cycle runs with short incubation time (20 s).

FIG. 7 shows polymerase performance comparisons in 150 cycle runs at short incorporation time.

FIGS. 8A to 8P shows amino acid sequences of SEQ ID NOs:1-32.

The schematic drawings are not necessarily to scale. Like numbers used in the figures refer to like components, steps and the like. However, it will be understood that the use of a number to refer to a component in a given figure is not intended to limit the component in another figure labeled with the same number. In addition, the use of different numbers to refer to components is not intended to indicate that the different numbered components cannot be the same or similar to other numbered components.

### DETAILED DESCRIPTION

### Polymerases

Provided herein are polymerases, compositions and kits that include a polymerase, and methods of using a polymerase. A polymerase described herein is a DNA polymerase. In one embodiment, a polymerase of the present disclosure, also referred to herein as an “altered polymerase,” is based on the amino acid sequence of a reference polymerase. An altered polymerase includes substitution mutations at one or more residues when compared to the reference polymerase. A substitution mutation can be at the same position or a functionally equivalent position compared to the reference polymerase. Reference polymerases and functionally equivalent positions are described herein. The skilled person will readily appreciate that altered polymerases of the present disclosure are not naturally occurring.

The altered polymerases described herein have useful activities compared to a reference polymerase. A reference polymerase described herein can be used in SBS reactions with modified nucleotides having a 3′-OH acetal blocking group or a 3′-OH thiocarbamate blocking group. Modified nucleotides having a 3′-OH acetal blocking group or a 3′-OH thiocarbamate blocking group are referred to herein as “second generation fully functional nucleotides,” or “second generation ffNs.” In one or more embodiments, a second generation ffNs can also include a cleavable linker. Second generation ffNs are described herein. Unless specifically noted otherwise, a description herein of incorporation of nucleotides by a polymerase is the incorporation of second generation ffNs.

A reference polymerase described herein has error rates that are useful in SBS reactions when using second generation ffNs and standard incorporation rates; however, using a reference polymerase in SBS reactions with fast incorporation times increases the error rate. Maintaining or surpassing current levels of performance at faster incorporation times can be aided by a new generation of polymerases. Presented herein are DNA polymerases having significantly improved performance under SBS fast cycle time conditions with second generation ffNs. The inventors have surprisingly identified certain altered polymerases which exhibit improved characteristics including improved accuracy during fast incorporations times with second generation ffNs. Improved accuracy includes reduced error rate and reduced phasing, and results in improved quality metrics in SBS reactions. Thus, altered polymerases of the present disclosure have one or more activities selected from lower error rate, lower phasing rate, and/or increased incorporation rate of second generation ffNs in comparison to a reference sequence, such as the reference sequence SEQ ID NO:1.

“Error rate” refers to a measurement of the frequency of error in the identification of the correct base, i.e., the complement of the template sequence at a specific position, during calls on a cluster during a sequencing reaction. The fidelity with which a sequenced library matches the original genome sequence can vary depending on the frequency of base mutation occurring at any stage from the extraction of the nucleic acid to its sequencing on a sequencing platform. This frequency places an upper limit on the probability of a sequenced base being correct. In some embodiments, the quality score is presented as a numerical value. For example, the quality score can be quoted as QXX where the XX is the score and it means that that particular call has a probability of error of 10^{-XX/10}. Thus, as an example, Q30 equates to an error rate of 1 in 1000, or 0.1%, and Q40 equates to an error rate of 1 in 10,000, or 0.01%.

Phasing is a term known to those of skill in the art and is used to describe the loss of synchrony in the readout of the sequence copies of a cluster. Phasing causes the extracted intensities for a specific cycle to include the signal of the current cycle and noise from the preceding cycle. Thus, as used herein, the term “phasing” refers to a phenomenon in SBS that is caused by incomplete incorporation of a nucleotide in some portion of DNA strands within clusters by polymerases at a given sequencing cycle, and is thus a measure of the rate at which single molecules within a cluster lose sync with each other. Phasing can be measured during detection of cluster signal at each cycle and can be reported as a percentage of detectable signal from a cluster that is out of synchrony with the signal in the cluster. As an example, a cluster is detected by a “green” fluorophore signal during cycle N. In the subsequent cycle (cycle N+1), 99.9% of the cluster signal is detected in the “red” channel and 0.1% of the signal remains from the previous cycle and is detected in the “green” channel. This result would indicate that phasing is occurring, and can be reported as a numerical value, such as a phasing value of 0.1, indicating that 0.1% of the molecules in the cluster are falling behind at each cycle.

Detection of phasing can be performed and reported according to any suitable methodology as is known in the art, for example, as described in U.S. Pat No. 8,965,076 and U.S. Provisional Patent No. 62/535,558. For example, as described in the Examples below, phasing is detected and reported routinely during SBS sequencing runs on sequencing instrument such as HiSeg™, Genome Analyzer™, NextSeg™, NextSeq 1000™, NextSeq 2000™, NovaSeg™, iSeg™, MiniSeg™, or MiSeg™ sequencing platforms from Illumina, Inc. (San Diego, CA) or any other suitable instrument known in the art.

Reduced cycle times can increase the occurrence of phasing, which contributes to error rate. The discovery of the mutations described herein of the altered polymerases which decrease the incidence of phasing when used in fast cycle time conditions with second generation ffNs was unexpected and provides a great advantage in SBS applications. For example, the altered polymerases can provide faster SBS cycle time with lower phasing, and optionally longer sequencing read length when using second generation ffNs. The characterization of error rate and phasing for altered polymerases as provided herein is set forth in the Example section below.

In one embodiment, reduced error rates occur in comparison to enzymes currently employed in SBS, such as SEQ ID NO:1 when the altered polymerase is tested using fast incorporation times. Incorporation refers to the amount of time a DNA polymerase is in contact with a template. As used herein, a slow incorporation time is the incorporation time used under a standard cycle using a MiniSeg™ benchtop sequencing system. Slow incorporation times include from 40 seconds to 50 seconds. As used herein, a fast cycle time refers to an incorporation step that is from 10 seconds to 40 seconds. In one embodiment, SBS fast cycle time conditions are an incorporation time of no greater than 40 seconds, no greater than 30 seconds, no greater than 20 seconds, no greater than 18 seconds, no greater than 16 seconds, no greater than 14 seconds, no greater than 12 seconds, or no greater than 10 seconds. In one embodiment, SBS fast cycle time conditions are an incorporation time of at least 10 seconds, at least 12 seconds, at least 14 seconds, at least 16 seconds, at least 18 seconds, at least 20 seconds, or at least 30 seconds.

An altered polymerase described herein can be used in SBS reactions for runs of different lengths. A “run” refers to the number of nucleotides that are identified on a template. A run typically includes a run based on the first primer (e.g., a readl primer) which reads one strand of a template and a run based on the second primer (e.g., a read2 primer) which reads the complementary strand of the template. In one embodiment, the number of nucleotides identified using the first primer or the second primer can be from 10 to 300 nucleotides. In one embodiment, the number of nucleotides identified using the first primer or the second primer can be no greater than 300 nucleotides, no greater than 250 nucleotides, no greater than 200 nucleotides, no greater than 150 nucleotides, no greater than 150 nucleotides, no greater than 130 nucleotides, no greater than 110 nucleotides, no greater than 90 nucleotides, no greater than 70 nucleotides, no greater than 50 nucleotides, no greater than 30 nucleotides, or no greater than 20 nucleotides. In one embodiment, the number of nucleotides identified using the first primer or the second primer can be at least 10, at least 20, at least 30, at least 50, at least 70, at least 90, at least 110, or at least 130 nucleotides.

In certain embodiments, an altered polymerase is based on a family B type DNA polymerase. An altered polymerase can be based on, for example, a family B archaeal DNA polymerase, a human DNA polymerase-a, or a phage polymerase. An altered polymerase of the present disclosure that is “based on” a family B type DNA polymerase means the altered polymerase is a family B type DNA polymerase that includes one or more of the substitution mutations described herein. An altered polymerase of the present disclosure that is “based on” a family B type DNA polymerase can also include one or more conservative and/or one or more nonconservative mutations as described herein.

Family B archaeal DNA polymerases are well known in the art as exemplified by the disclosure of U.S. Pat. No. 8,460,910. In certain embodiments, altered polymerase of the present disclosure is based on a family B archaeal DNA polymerase is from a hyperthermophilic archaeon and is thermostable.

In certain embodiments, a family B archaeal DNA polymerase is from a genus such as, for example, Thermococcus, Pyrococcus, Methanococcus, Pyrobaculum, Pyrodictium, and Aeropyrum. Members of the genus Thermococcus are well known in the art and include, but are not limited to T. 4557, T. barophilus, T. gammatolerans, T. onnurineus, T. sibiricus, T. kodakarensis, T. gorgonarius (TGO), and T. waiotapuensis. Members of the genus Pyrococcus are well known in the art and include, but are not limited to P. NA2, P. abyssi, P. furiosus, P. horikoshii, P. yayanosii, P. endeavori, P. glycovorans, and P. woesei. Members of the genus Methanococcus are well known in the art and include, but are not limited to M. aeolicus, M. maripaludis, M. vannielii, M. voltae, M. thermolithotrophicus, and M. jannaschii. Members of the genus Pyrobaculum are well known in the art and include, but are not limited to, P. calidifontis (Pc). Members of the genus Pyrodictium are well known in the art and include, but are not limited to, P. occultum. Members of the genus Aeropyrum are well known in the art and include, but are not limited to, A. pernix.

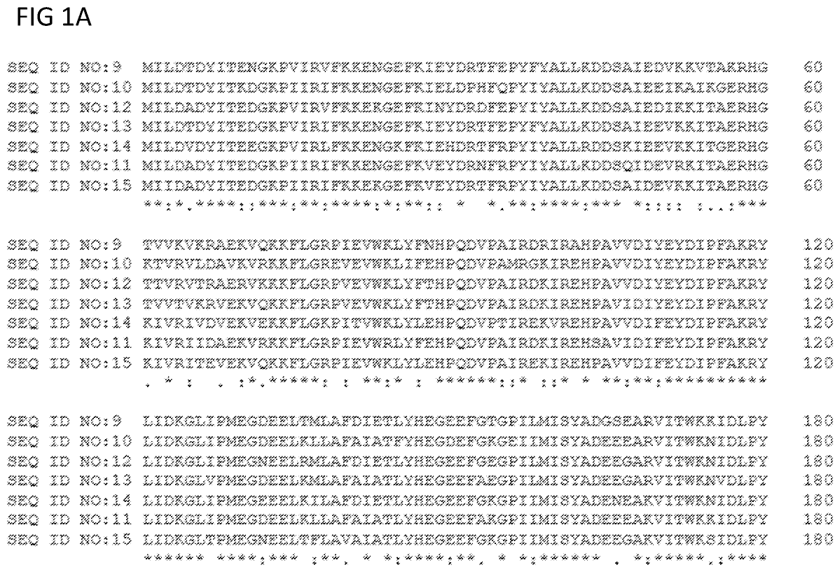

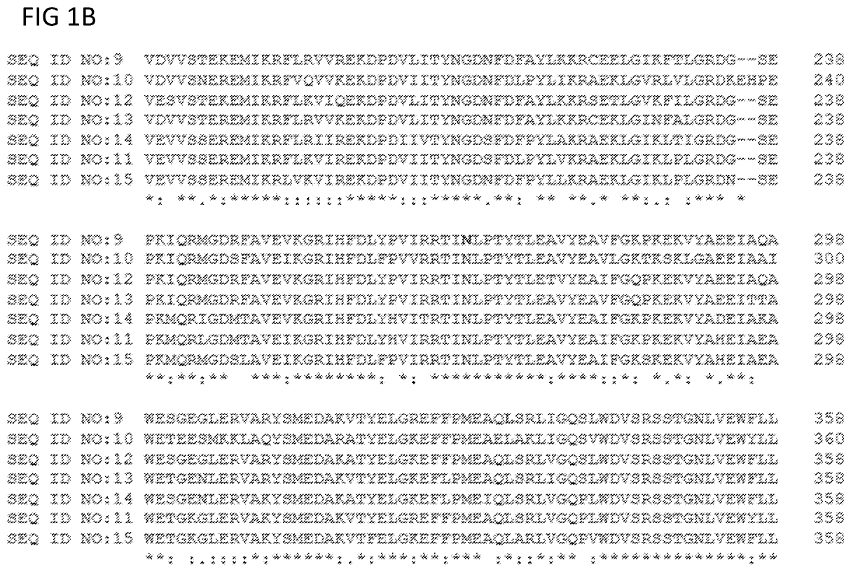

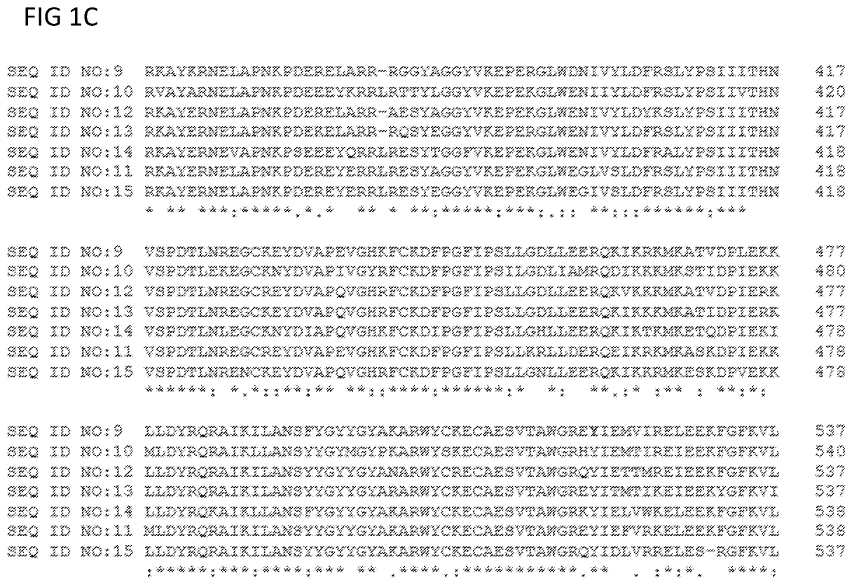

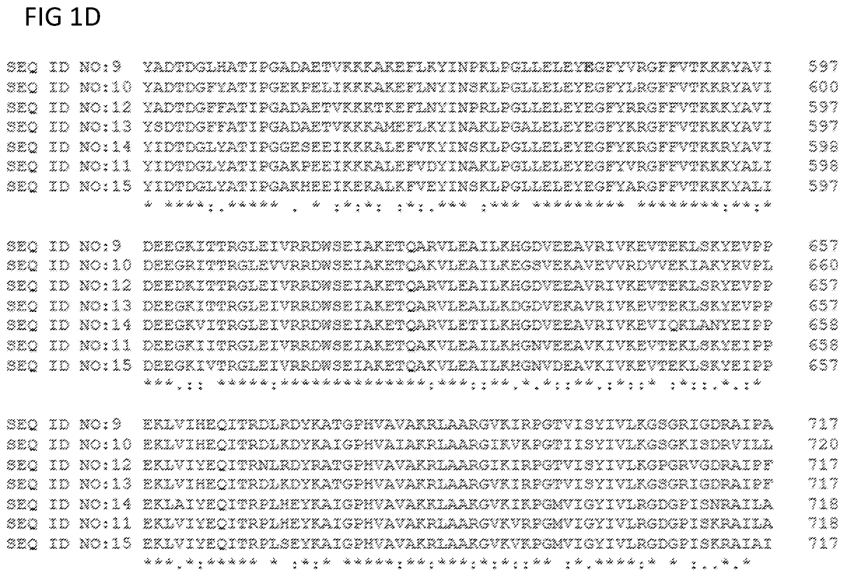

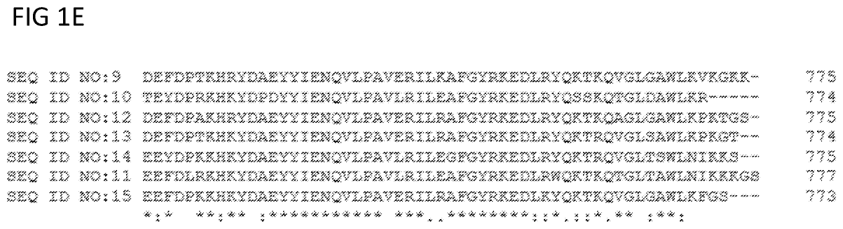

In one embodiment an altered family B DNA polymerase is based on Vent®, Deep Vent®, 9° N, Pfu, KOD, or a Pab polymerase. Vent® and Deep Vent® are commercial names used for family B DNA polymerases isolated from the hyperthermophilic archaeon Thermococcus litoralis. 9° N polymerase is a family B polymerase isolated from Thermococcus sp. 9° N-7. Pfu polymerase is a family B polymerase isolated from Pyrococcus furiosus. KOD polymerase is a family B polymerase isolated from Thermococcus kodakarensis. Pab polymerase is a family B polymerase isolated from Pyrococcus abyssi. Twa is a family B polymerase isolated from T. waiotapuensis. Examples of Vent®, Deep Vent®, 9° N, Pfu, KOD, Pab, and Twa polymerases are disclosed in FIG. 1.

In certain embodiments, an altered polymerase is based on a family B DNA polymerase from a phage such as, for example, T4, RB69, or phi29 phage.

FIG. 1 shows a sequence alignment for proteins having the amino acid sequences shown in SEQ ID NOs:9-15. The alignment indicates amino acids that are conserved in the different family B polymerases. The skilled person will appreciate that the conserved amino acids and conserved regions are most likely conserved because they are important to the function of the polymerases, and therefore show a correlation between structure and function of the polymerases. The alignment also shows regions of variability across the different family B polymerases. A person of ordinary skill in the art can deduce from such data regions of a polymerase in which substitutions, particularly conservative substitutions, may be permitted without unduly affecting biological activity of the altered polymerase.

An altered polymerase described herein can be based on the amino acid sequence of a known polymerase (also referred to herein as a reference polymerase) and further includes substitution mutations at one or more residues. In one embodiment, a substitution mutation is at a position functionally equivalent to an amino acid of a reference polymerase. By “functionally equivalent” it is meant that the altered polymerase has the amino acid substitution at the amino acid position in the reference polymerase that has the same functional role in both the reference polymerase and the altered polymerase.

In general, functionally equivalent substitution mutations in two or more different polymerases occur at homologous amino acid positions in the amino acid sequences of the polymerases. Hence, use herein of the term “functionally equivalent” also encompasses mutations that are “positionally equivalent” or “homologous” to a given mutation, regardless of whether or not the particular function of the mutated amino acid is known. It is possible to identify the locations of functionally equivalent and positionally equivalent amino acid residues in the amino acid sequences of two or more different polymerases on the basis of sequence alignment and/or molecular modelling. An example of sequence alignment to identify positionally equivalent and/or functionally equivalent residues is set forth in FIG. 1. For example, the residues in the Twa, KOD, Pab, Pfu, Deep Vent, and Vent polymerases of FIG. 1 that are vertically aligned are considered positionally equivalent as well as functionally equivalent to the corresponding residue in the 9° N polymerase amino acid sequence. Thus, for example residue 358 of the 9° N, Twa, KOD, Pfu, Deep Vent, and Pab polymerases and residue 360 of the Vent polymerase are functionally equivalent and positionally equivalent. Likewise, for example residue 633 of the 9° N, Twa, KOD, and Pab polymerases, residue 634 of the Pfu and Deep Vent polymerases, and residue 636 of the Vent polymerase are functionally equivalent and positionally equivalent. The skilled person can easily identify functionally equivalent residues in DNA polymerases.

In certain embodiments, the substitution mutation comprises a mutation to a residue having a non-polar side chain. Amino acids having non-polar side chains are well-known in the art and include, for example: alanine, glycine, isoleucine, leucine, methionine, phenylalanine, proline, tryptophan, and valine.

In certain embodiments, the substitution mutation comprises a mutation to a residue having a polar side chain. Amino acids having polar side chains are well-known in the art and include, for example: arginine, asparagine, aspartic acid, glutamine, glutamic acid, histidine, lysine, serine, cysteine, tyrosine, and threonine.

In certain embodiments, the substitution mutation comprises a mutation to a residue having a hydrophobic side chain. Amino acids having hydrophobic side chains are well-known in the art and include, for example: glycine, alanine, valine, leucine, isoleucine, proline, phenylalanine, methionine, and tryptophan.

In certain embodiments, the substitution mutation comprises a mutation to a residue having an uncharged side chain. Amino acids having uncharged side chains are well-known in the art and include, for example: glycine, serine, cysteine, asparagine, glutamine, tyrosine, and threonine.

In one or more embodiments, an altered polymerase has an amino acid sequence that is structurally similar to a reference polymerase disclosed herein. In one embodiment, a reference polymerase is one that includes the amino acid sequence of the archaeal family B DNA polymerase 1901, also referred to here as Pol 1901 (SEQ ID NO:1). Other reference sequences include SEQ ID NO:9, 10, 11, 12, 13, 14, and 15with substitution mutations functionally equivalent to the following substitution mutations in SEQ ID NO:1: Met129Ala, Aspl41 Ala, Glul43 Ala, Cys223Ser, Arg247Tyr, Thr349Lys, Leu408Ala, Tyr409Ala, Pro410Ile, Ala485Val, Tyr497Gly, Glu599Asp, and His633Gly.

As used herein, an altered polymerase may be “structurally similar” to a reference polymerase if the amino acid sequence of the altered polymerase possesses a specified amount of sequence similarity and/or sequence identity compared to the reference polymerase.

Structural similarity of two amino acid sequences can be determined by aligning the residues of the two sequences (for example, a candidate polymerase and a reference polymerase described herein) to optimize the number of identical amino acids along the lengths of their sequences; gaps in either or both sequences are permitted in making the alignment in order to optimize the number of identical amino acids, although the amino acids in each sequence must nonetheless remain in their proper order. A candidate polymerase is the polymerase being compared to the reference polymerase. A candidate polymerase that has structural similarity with a reference polymerase and polymerase activity is an altered polymerase.

A pair-wise comparison analysis of amino acid sequences or nucleotide sequences can be conducted, for instance, by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Current Protocols in Molecular Biology, Ausubel et al., eds., Current Protocols, a joint venture between Greene Publishing Associates, Inc. and John Wiley & Sons, Inc., supplemented through 2004).

Unless modified as otherwise described herein, the algorithm used to determine structural similarity is the BLAST algorithm, which is described in Altschul et al., J. Mol. Biol. 215:403-410 (1990). Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., J. Mol. Biol. 215:403-410 (1990)). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff (1989) Proc. Natl. Acad. Sci. USA 89:10915).

In addition to calculating percent sequence identity, the BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more preferably less than about 0.01, and most preferably less than about 0.001.

In the comparison of two amino acid sequences, structural similarity may be referred to by percent “identity” or may be referred to by percent “similarity.” “Identity” refers to the presence of identical amino acids. “Similarity” refers to the presence of not only identical amino acids but also the presence of conservative substitutions. A conservative substitution for an amino acid in a protein may be selected from other members of the class to which the amino acid belongs. For example, it is well-known in the art of protein biochemistry that an amino acid belonging to a grouping of amino acids having a particular size or characteristic (such as charge, hydrophobicity, or hydrophilicity) can be substituted for another amino acid without altering the activity of a protein, particularly in regions of the protein that are not directly associated with biological activity. For example, non-polar amino acids include alanine, glycine, isoleucine, leucine, methionine, phenylalanine, proline, tryptophan, and valine. Hydrophobic amino acids include glycine, alanine, valine, leucine, isoleucine, proline, phenylalanine, methionine, and tryptophan. Polar amino acids include arginine, asparagine, aspartic acid, glutamine, glutamic acid, histidine, lysine, serine, cysteine, tyrosine, and threonine. The uncharged amino acids include glycine, serine, cysteine, asparagine, glutamine, tyrosine, and threonine, among others.

Thus, as used herein, reference to a polymerase as described herein, such as reference to the amino acid sequence of one or more SEQ ID NOs described herein can include a protein with at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% amino acid sequence similarity to the reference polymerase.

Alternatively, as used herein, reference to a polymerase as described herein, such as reference to the amino acid sequence of one or more SEQ ID NOs described herein can include a protein with at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% amino acid sequence identity to the reference polymerase.

The present disclosure describes mutations that result in a polymerase having one or more of the polymerase activities described herein. A polymerase described herein can include any number of mutations, e.g., at least 1, at least 2, at least 3, at least 4, or at least 5 mutations compared to a reference polymerase, such as SEQ ID NO:1. Likewise, a polymerase described herein can include the mutations in any combination.

In one or more embodiments, an altered family B polymerase described herein includes a substitution mutation at a position functionally equivalent to R58, Y261, N269, F283, P328, M329, Q332, L333, S347, N399, F405, R406, 1410, 1412, E458, E459, Q461, A469, Y481, V485, 1486, W504, K507, E511, W516, Y520, 1521, M523, R526, E527, 1567, N568, L571, E576, E580, T590, A595, or 1603 in Pol 1901 (SEQ ID NO:1).

In one embodiment, an altered family B polymerase includes a substitution mutation at a position functionally equivalent to Arg58 in Pol 1901 (SEQ ID NO:1). In one embodiment, the substitution mutation at a position functionally equivalent to Arg58 is a mutation to a non-polar or hydrophobic amino acid, for example Leu.

In one embodiment, an altered family B polymerase includes a substitution mutation at a position functionally equivalent to Tyr261 in Pol 1901 (SEQ ID NO:1). In one embodiment, the substitution mutation at a position functionally equivalent to Tyr261 is a mutation to a non-polar, hydrophobic, or uncharged amino acid, for example Gly.

In one embodiment, an altered family B polymerase includes a substitution mutation at a position functionally equivalent to Asn269 in Pol 1901 (SEQ ID NO:1). In one embodiment, the substitution mutation at a position functionally equivalent to Asn269 is a mutation to a non-polar amino acid, for example Gly or Val. In one embodiment, the substitution mutation at a position functionally equivalent to Asn269 is a mutation to a hydrophobic amino acid, for example Gly or Val. In one embodiment, the substitution mutation at a position functionally equivalent to Asn269 is a mutation to an uncharged amino acid, for example Gly.

In one embodiment, an altered family B polymerase includes a substitution mutation at a position functionally equivalent to Phe283 in Pol 1901 (SEQ ID NO:1). In one embodiment, the substitution mutation at a position functionally equivalent to Phe283 is a mutation to a non-polar, hydrophobic, or uncharged amino acid, for example Ile. In one embodiment, the substitution mutation at a position functionally equivalent to Phe283 is a mutation to a polar for example Lys.

In one embodiment, an altered family B polymerase includes a substitution mutation at a position functionally equivalent to Pro328 in Pol 1901 (SEQ ID NO:1). In one embodiment, the substitution mutation at a position functionally equivalent to Pro328 is a mutation to a polar or uncharged amino acid, for example Asp.

In one embodiment, an altered family B polymerase includes a substitution mutation at a position functionally equivalent to Met329 in Pol 1901 (SEQ ID NO:1). In one embodiment, the substitution mutation at a position functionally equivalent to Met329 is a mutation to a polar or uncharged amino acid, for example Thr.

In one embodiment, an altered family B polymerase includes a substitution mutation at a position functionally equivalent to Gln332 in Pol 1901 (SEQ ID NO:1). In one embodiment, the substitution mutation at a position functionally equivalent to Gln332 is a mutation to a polar or uncharged amino acid, for example Ser.

In one embodiment, an altered family B polymerase includes a substitution mutation at a position functionally equivalent to Leu333 in Pol 1901 (SEQ ID NO:1). In one embodiment, the substitution mutation at a position functionally equivalent to Leu333 is a mutation to a polar amino acid, for example His. In one embodiment, the substitution mutation at a position functionally equivalent to Leu333 is a mutation to a non-polar amino acid, for example Ile. In one embodiment, the substitution mutation at a position functionally equivalent to Leu333 is a mutation to a hydrophobic amino acid, for example Ile.

In one embodiment, an altered family B polymerase includes a substitution mutation at a position functionally equivalent to S347 in Pol 1901 (SEQ ID NO:1). In one embodiment, the substitution mutation at a position functionally equivalent to S347 is a mutation to a polar amino acid, for example Asp, Arg, or Glu. In one embodiment, the substitution mutation at a position functionally equivalent to S347 is a mutation to a non-polar amino acid, for example Val. In one embodiment, the substitution mutation at a position functionally equivalent to S347 is a mutation to a hydrophobic amino acid, for example Val. In one embodiment, the substitution mutation at a position functionally equivalent to S347 is a mutation to a polar or uncharged amino acid, for example Thr.

In one embodiment, an altered family B polymerase includes a substitution mutation at a position functionally equivalent to Asn399 in Pol 1901 (SEQ ID NO:1). In one embodiment, the substitution mutation at a position functionally equivalent to Asn399 is a mutation to a non-polar amino acid, for example Ala or Met. In one embodiment, the substitution mutation at a position functionally equivalent to Asn399 is a mutation to a hydrophobic amino acid, for example Ala or Met.

In one embodiment, an altered family B polymerase includes a substitution mutation at a position functionally equivalent to Phe405 in Pol 1901 (SEQ ID NO:1). In one embodiment, the substitution mutation at a position functionally equivalent to Phe405 is a mutation to a non-polar amino acid or hydrophobic amino acid, for example Met. In one embodiment, the substitution mutation at a position functionally equivalent to Phe405 is a mutation to a polar amino acid, for example Lys and Gln. In one embodiment, the substitution mutation at a position functionally equivalent to Phe405 is a mutation to an uncharged amino acid, for example Gln. In one embodiment, the substitution mutation at a position functionally equivalent to Phe405 is a mutation to Met. In one embodiment, the substitution mutation at a position functionally equivalent to Phe405 is a mutation to Lys.

In one embodiment, an altered family B polymerase includes a substitution mutation at a position functionally equivalent to Arg406 in Pol 1901 (SEQ ID NO:1). In one embodiment, the substitution mutation at a position functionally equivalent to Arg406 is a mutation to a non-polar or hydrophobic amino acid, for example Met.

In one embodiment, an altered family B polymerase includes a substitution mutation at a position functionally equivalent to Ile410 in Pol 1901 (SEQ ID NO:1). In one embodiment, the substitution mutation at a position functionally equivalent to Ile410 is a mutation to a polar or uncharged amino acid, for example Ser.

In one embodiment, an altered family B polymerase includes a substitution mutation at a position functionally equivalent to Ile412 in Pol 1901 (SEQ ID NO:1). In one embodiment, the substitution mutation at a position functionally equivalent to Ile412 is a mutation to a non-polar amino acid, for example Val. In one embodiment, the substitution mutation at a position functionally equivalent to Ile412 is a mutation to a hydrophobic amino acid, for example Val.

In one embodiment, an altered family B polymerase includes a substitution mutation at a position functionally equivalent to Glu458 in Pol 1901 (SEQ ID NO:1). In one embodiment, the substitution mutation at a position functionally equivalent to Glu458 is a mutation to a non-polar, hydrophobic, or uncharged amino acid, for example Gly.

In one embodiment, an altered family B polymerase includes a substitution mutation at a position functionally equivalent to Glu459 in Pol 1901 (SEQ ID NO:1). In one embodiment, the substitution mutation at a position functionally equivalent to Glu459 is a mutation to a polar or uncharged amino acid, for example Thr. In one embodiment, the substitution mutation at a position functionally equivalent to Glu459 is a mutation to a polar or uncharged amino acid, for example Asp.

In one embodiment, an altered family B polymerase includes a substitution mutation at a position functionally equivalent to Gln461 in Pol 1901 (SEQ ID NO:1). In one embodiment, the substitution mutation at a position functionally equivalent to Gln461 is a mutation to a polar amino acid, for example Tyr. In one embodiment, the substitution mutation at a position functionally equivalent to Gln461 is a mutation to an uncharged amino acid, for example Tyr.

In one embodiment, an altered family B polymerase includes a substitution mutation at a position functionally equivalent to Ala469 in Pol 1901 (SEQ ID NO:1). In one embodiment, the substitution mutation at a position functionally equivalent to Ala469 is a mutation to a polar amino acid, for example Glu.

In one embodiment, an altered family B polymerase includes a substitution mutation at a position functionally equivalent to Tyr481 in Pol 1901 (SEQ ID NO:1). In one embodiment, the substitution mutation at a position functionally equivalent to Tyr481 is a mutation to a non-polar amino acid, for example Ile.

In one embodiment, an altered family B polymerase includes a substitution mutation at a position functionally equivalent to Val485 in Pol 1901 (SEQ ID NO:1). In one embodiment, the substitution mutation at a position functionally equivalent to Val485 is a mutation to a non-polar or hydrophobic amino acid, for example Met or Gly, preferably Gly. In one embodiment, the substitution mutation at a position functionally equivalent to Val485 is a mutation to a polar amino acid, for example Lys, Asn, Gln, Ser, or Thr, preferably Asn or Lys. In one embodiment, the substitution mutation at a position functionally equivalent to Val485 is a mutation to a hydrophobic amino acid, for example Gly. In one embodiment, the substitution mutation at a position functionally equivalent to Val485 is a mutation to an uncharged amino acid, for example Asn, Gly, Gln, Ser, or Thr.

In one embodiment, an altered family B polymerase includes a substitution mutation at a position functionally equivalent to Ile486 in Pol 1901 (SEQ ID NO:1). In one embodiment, the substitution mutation at a position functionally equivalent to Ile486 is a mutation to a non-polar amino acid, for example Leu. In one embodiment, the substitution mutation at a position functionally equivalent to Ile486 is a mutation to a hydrophobic amino acid, for example Leu.

In one embodiment, an altered family B polymerase includes a substitution mutation at a position functionally equivalent to Trp504 in Pol 1901 (SEQ ID NO:1). In one embodiment, the substitution mutation at a position functionally equivalent to Trp504 is a mutation to a polar amino acid, for example Asn. In one embodiment, the substitution mutation at a position functionally equivalent to Trp504 is a mutation to an uncharged amino acid, for example Asn.

In one embodiment, an altered family B polymerase includes a substitution mutation at a position functionally equivalent to Lys507 in Pol 1901 (SEQ ID NO:1). In one embodiment, the substitution mutation at a position functionally equivalent to Lys507 is a mutation to a polar amino acid, for example His. In one embodiment, the substitution mutation at a position functionally equivalent to Lys507 is a mutation to a non-polar or hydrophobic amino acid, for example Pro.

In one embodiment, an altered family B polymerase includes a substitution mutation at a position functionally equivalent to Glu511 in Pol 1901 (SEQ ID NO:1). In one embodiment, the substitution mutation at a position functionally equivalent to Glu511 is a mutation to a polar amino acid, for example Arg or Lys. In one embodiment, the substitution mutation at a position functionally equivalent to Glu511 is a mutation to a non-polar amino acid, for example Met. In one embodiment, the substitution mutation at a position functionally equivalent to Glu511 is a mutation to a hydrophobic amino acid, for example Met.

In one embodiment, an altered family B polymerase includes a substitution mutation at a position functionally equivalent to Trp516 in Pol 1901 (SEQ ID NO:1). In one embodiment, the substitution mutation at a position functionally equivalent to Trp516 is a mutation to a polar amino acid, for example Gln or Lys. In one embodiment, the substitution mutation at a position functionally equivalent to Trp516 is a mutation to a non-polar or hydrophobic amino acid, for example Ile or Met. In one embodiment, the substitution mutation at a position functionally equivalent to Trp516 is a mutation to a hydrophobic amino acid, for example Ile. In one embodiment, the substitution mutation at a position functionally equivalent to Trp516 is a mutation to an uncharged amino acid, for example Gln. In one embodiment, the substitution mutation at a position functionally equivalent to Trp516 is a mutation to an uncharged amino acid, for example Leu.

In one embodiment, an altered family B polymerase includes a substitution mutation at a position functionally equivalent to Tyr520 in Pol 1901 (SEQ ID NO:1). In one embodiment, the substitution mutation at a position functionally equivalent to Tyr520 is a mutation to a non-polar amino acid, for example Ala. In one embodiment, the substitution mutation at a position functionally equivalent to Tyr520 is a mutation to a hydrophobic amino acid, for example Ala.

In one embodiment, an altered family B polymerase includes a substitution mutation at a position functionally equivalent to Ile521 in Pol 1901 (SEQ ID NO:1). In one embodiment, the substitution mutation at a position functionally equivalent to Ile521 is a mutation to a polar or uncharged amino acid, for example Thr.

In one embodiment, an altered family B polymerase includes a substitution mutation at a position functionally equivalent to Met523 in Pol 1901 (SEQ ID NO:1). In one embodiment, the substitution mutation at a position functionally equivalent to Met523 is a mutation to a polar or uncharged amino acid, for example Thr. In one embodiment, the substitution mutation at a position functionally equivalent to Met523 is a mutation to a non-polar or hydrophobic amino acid, for example Ile.

In one embodiment, an altered family B polymerase includes a substitution mutation at a position functionally equivalent to Arg526 in Pol 1901 (SEQ ID NO:1). In one embodiment, the substitution mutation at a position functionally equivalent to Arg526 is a mutation to a polar or uncharged amino acid, for example Asn.

In one embodiment, an altered family B polymerase includes a substitution mutation at a position functionally equivalent to Glu527 in Pol 1901 (SEQ ID NO:1). In one embodiment, the substitution mutation at a position functionally equivalent to Glu527 is a mutation to a non-polar or hydrophobic amino acid, for example Ile.

In one embodiment, an altered family B polymerase includes a substitution mutation at a position functionally equivalent to Leu528 in Pol 1901 (SEQ ID NO:1). In one embodiment, the substitution mutation at a position functionally equivalent to Leu528 is a mutation to a polar or uncharged amino acid, for example Thr.

In one embodiment, an altered family B polymerase includes a substitution mutation at a position functionally equivalent to Ile567 in Pol 1901 (SEQ ID NO:1). In one embodiment, the substitution mutation at a position functionally equivalent to Ile567 is a mutation to a non-polar amino acid, for example Leu. In one embodiment, the substitution mutation at a position functionally equivalent to Ile567 is a mutation to a hydrophobic amino acid, for example Leu.

In one embodiment, an altered family B polymerase includes a substitution mutation at a position functionally equivalent to Asn568 in Pol 1901 (SEQ ID NO:1). In one embodiment, the substitution mutation at a position functionally equivalent to Asn568 is a mutation to a polar amino acid, for example Gln. In one embodiment, the substitution mutation at a position functionally equivalent to Asn568 is a mutation to an uncharged amino acid, for example Gln.

In one embodiment, an altered family B polymerase includes a substitution mutation at a position functionally equivalent to Leu571 in Pol 1901 (SEQ ID NO:1). In one embodiment, the substitution mutation at a position functionally equivalent to Leu571 is a mutation to a non-polar or hydrophobic amino acid, for example Phe, Ile, Met, or Trp.

In one embodiment, an altered family B polymerase includes a substitution mutation at a position functionally equivalent to Glu576 in Pol 1901 (SEQ ID NO:1). In one embodiment, the substitution mutation at a position functionally equivalent to Glu576 is a mutation to a non-polar or hydrophobic amino acid, for example Leu.

In one embodiment, an altered family B polymerase includes a substitution mutation at a position functionally equivalent to Glu580 in Pol 1901 (SEQ ID NO:1). In one embodiment, the substitution mutation at a position functionally equivalent to Glu580 is a mutation to a polar amino acid, for example Gln, Lys, or Arg, preferably Lys. In one embodiment, the substitution mutation at a position functionally equivalent to Glu580 is a mutation to a non-polar amino acid, for example Ile, Val, or Met. In one embodiment, the substitution mutation at a position functionally equivalent to Glu580 is a mutation to a hydrophobic amino acid, for example Ile or Val. In one embodiment, the substitution mutation at a position functionally equivalent to Glu580 is a mutation to an uncharged amino acid, for example Gln. In one embodiment, the substitution mutation at a position functionally equivalent to Glu580 is a mutation to Arg. In one embodiment, the substitution mutation at a position functionally equivalent to Glu580 is a mutation to Ile. In one embodiment, the substitution mutation at a position functionally equivalent to Glu580 is a mutation to Lys.

In one embodiment, an altered family B polymerase includes a substitution mutation at a position functionally equivalent to Thr590 in Pol 1901 (SEQ ID NO:1). In one embodiment, the substitution mutation at a position functionally equivalent to Thr590 is a mutation to a polar amino acid, for example Lys.

In one embodiment, an altered family B polymerase includes a substitution mutation at a position functionally equivalent to Ala595 in Pol 1901 (SEQ ID NO:1). In one embodiment, the substitution mutation at a position functionally equivalent to Ala595 is a mutation to a non-polar or hydrophobic amino acid, for example Met.

In one embodiment, an altered family B polymerase includes a substitution mutation at a position functionally equivalent to Ile603 in Pol 1901 (SEQ ID NO:1). In one embodiment, the substitution mutation at a position functionally equivalent to Ile603 is a mutation to a polar amino acid, for example Asp.

The present disclosure also provides altered family B polymerases having combinations of the substitution mutations including, but not limited to, those described hereinabove. In one or more embodiments, an altered family B polymerase includes substitution mutation at least two, three, four, five, or six positions.

In one embodiment, an altered polymerase includes at least two substitution mutations. The first substitution mutation is at a position functionally equivalent to Glu580 in Pol 1901 (SEQ ID NO:1), where the substitution mutation at a position functionally equivalent to Glu580 is a mutation to a polar amino acid, for example Gln, Lys, or Arg, preferably Lys. The second substitution mutation can beat a position functionally equivalent to Trp516 in Pol 1901, where the substitution mutation at a position functionally equivalent to Trp516 is a mutation to a non-polar amino acid, for example Ile or Met, preferably Met. Alternatively, the second substitution mutation can be at a position functionally equivalent to Trp516 in Pol 1901, where the substitution mutation at a position functionally equivalent to Trp516 is a mutation to a non-polar or hydrophobic amino acid, for example Leu. In one embodiment, the substitution mutation a position functionally equivalent to Glu580 is a mutation to Lys and the substitution mutation a position functionally equivalent to Trp516 is a mutation to Met or to Leu.

In one embodiment, an altered polymerase includes at least two substitution mutations. A first substitution mutation is at a position functionally equivalent to Glu580 in Pol 1901 (SEQ ID NO:1), where the substitution mutation at a position functionally equivalent to Glu580 is a mutation to a polar amino acid, for example Gln, Lys, or Arg, preferably Lys. The second substitution mutation is at a position functionally equivalent to Ala408 in Pol 1901, where the substitution mutation at a position functionally equivalent to Ala408 is a mutation to a polar or uncharged amino acid, for example Ser. In one embodiment, the substitution mutation at a position functionally equivalent to Glu580 is a mutation to Lys and the substitution mutation at a position functionally equivalent to Ala408 is a mutation to Ser.

In one embodiment, an altered polymerase includes at least two substitution mutations. A first substitution mutation is at a position functionally equivalent to Phe405 in Pol 1901 (SEQ ID NO:1), where the substitution mutation at a position functionally equivalent to Phe405 is a mutation to a non-polar amino acid, for example Met. The second substitution mutation is at a position functionally equivalent to Val485 in Pol 1901, where the substitution mutation at a position functionally equivalent to Val485 is a mutation to a non-polar, hydrophobic, or uncharged amino acid, for example Gly. In one embodiment, the substitution mutation at a position functionally equivalent to Val485 is a mutation to Met and the substitution mutation is at a position functionally equivalent to Val485 is a mutation to Gly.

In one embodiment, an altered polymerase includes at least two substitution mutations. A first substitution mutation is at a position functionally equivalent to Leu571 in Pol 1901 (SEQ ID NO:1), where the substitution mutation at a position functionally equivalent to Leu571 is a mutation to a non-polar or hydrophobic amino acid, for example Phe, Ile, Met, or Trp. The second substitution mutation is at a position functionally equivalent to Glu580 in Pol 1901, where the substitution mutation at a position functionally equivalent to Glu580 is a mutation to a polar amino acid, for example Gln, Lys, or Arg. In one embodiment, the substitution mutation a position functionally equivalent to Leu571 is a mutation to Phe and the substitution mutation a position functionally equivalent to Glu580 is a mutation to Lys. In another embodiment, the substitution mutation at a position functionally equivalent to Leu571 is a mutation to Met and the substitution mutation at a position functionally equivalent to Glu580 is a mutation to Gln.

In one embodiment, an altered polymerase includes at least two substitution mutations. A first substitution mutation is at a position functionally equivalent to Val485 in Pol 1901 (SEQ ID NO:1), where the substitution mutation at a position functionally equivalent to Val485 is a mutation to a polar amino acid, for example Lys, Asn, Gln, Ser, or Thr, or an uncharged amino acid, for example Asn, Gly, Gln, Ser, or Thr. The second substitution mutation is at a position functionally equivalent to Glu580 in Pol 1901, where the substitution mutation at a position functionally equivalent to Glu580 is a mutation to a polar amino acid, for example Gln, Lys, or Arg. In one embodiment, the substitution mutation at a position functionally equivalent to Val485 is a mutation to Thr and the substitution mutation at a position functionally equivalent to Glu580 is a mutation to Lys.

In one embodiment, an altered polymerase includes at least two substitution mutations. A first substitution mutation is at a position functionally equivalent to Ile410 in Pol 1901 (SEQ ID NO:1), where the substitution mutation at a position functionally equivalent to Ile410 is a mutation to a non-polar or hydrophobic amino acid, for example Val. The second substitution mutation is at a position functionally equivalent to Glu580 in Pol 1901, where the substitution mutation at a position functionally equivalent to Glu580 is a mutation to a polar amino acid, for example Gln, Lys, or Arg. In one embodiment, the substitution mutation at a position functionally equivalent to Ile410 is a mutation to Val and the substitution mutation at a position functionally equivalent to Glu580 is a mutation to Lys.

In one embodiment, an altered polymerase includes at least two substitution mutations. A first substitution mutation is at a position functionally equivalent to Phe405 in Pol 1901, where the substitution mutation at a position functionally equivalent to Phe405 is a mutation to a non-polar amino acid or hydrophobic amino acid, for example Met. The second substitution mutation is at a position functionally equivalent to Ile410 in Pol 1901 (SEQ ID NO:1), where the substitution mutation at a position functionally equivalent to Ile410 is a mutation to a non-polar or hydrophobic amino acid, for example Val. In one embodiment, the substitution mutation at a position functionally equivalent to Phe405 is a mutation to Met, and the substitution mutation at a position functionally equivalent to Ile410 is a mutation to Val.

In one embodiment, an altered polymerase includes at least two substitution mutations. A first substitution mutation is at a position functionally equivalent to Phe 140 in Pol 1901, where the substitution mutation at a position functionally equivalent to Phe140 is a mutation to a non-polar amino acid or hydrophobic amino acid, for example Leu. The second substitution mutation is at a position functionally equivalent to Ser407 in Pol 1901 (SEQ ID NO:1), where the substitution mutation at a position functionally equivalent to Ser407 is a mutation to a non-polar amino acid or hydrophobic amino acid, for example Leu. In one embodiment, the substitution mutation at a position functionally equivalent to Phe 140 is a mutation to Leu, and the substitution mutation at a position functionally equivalent to Ser407 is a mutation to Leu.

In one embodiment, an altered polymerase includes at least two substitution mutations. A first substitution mutation is at a position functionally equivalent to Phe405 in Pol 1901, where the substitution mutation at a position functionally equivalent to Phe405 is a mutation to a non-polar amino acid or hydrophobic amino acid, for example Met. The second substitution mutation is at a position functionally equivalent to Glu580 in Pol 1901, where the substitution mutation at a position functionally equivalent to Glu580 is a mutation to a polar amino acid, for example Gln, Lys, or Arg. In one embodiment, the substitution mutation at a position functionally equivalent to Phe405 is a mutation to Met, and the substitution mutation at a position functionally equivalent to Glu580 is a mutation to Arg.

In one embodiment, an altered polymerase includes at least two substitution mutations. A first substitution mutation is at a position functionally equivalent to Phe405 in Pol 1901, where the substitution mutation at a position functionally equivalent to Phe405 is a mutation to a non-polar amino acid or hydrophobic amino acid, for example Met. The second substitution mutation is at a position functionally equivalent to Glu580 in Pol 1901, where the substitution mutation at a position functionally equivalent to Glu580 is a mutation to a polar amino acid, for example Gln, Lys, or Arg. In one embodiment, the substitution mutation at a position functionally equivalent to Phe405 is a mutation to Met, and the substitution mutation at a position functionally equivalent to Glu580 is a mutation to Lys.

In one embodiment, an altered polymerase includes at least three substitution mutations. A first substitution mutation is at a position functionally equivalent to Glu580 in Pol 1901 (SEQ ID NO:1), where the substitution mutation at a position functionally equivalent to Glu580 is a mutation to a polar amino acid, for example Gln, Lys, or Arg, preferably Lys. The second substitution mutation is at a position functionally equivalent to Ala408 in Pol 1901, where the substitution mutation at a position functionally equivalent to Ala408 is a mutation to a polar or uncharged amino acid, for example Ser. The third substitution mutation is at a position functionally equivalent to Ile410 in Pol 1901, where the substitution mutation at a position functionally equivalent to Ile410 is a mutation to a non-polar or hydrophobic amino acid, for example Val. In one embodiment, the substitution mutation at a position functionally equivalent to Glu580 is a mutation to Lys, the substitution mutation at a position functionally equivalent to Ala408 is a mutation to Ser, and the substitution mutation at a position functionally equivalent to Ile410 is a mutation to Val.

In one embodiment, an altered polymerase includes at least three substitution mutations. A first substitution mutation is at a position functionally equivalent to Phe405 in Pol 1901 (SEQ ID NO:1), where the substitution mutation at a position functionally equivalent to Phe405 is a mutation to a non-polar amino acid, for example Met. The second substitution mutation is at a position functionally equivalent to Val485 in Pol 1901, where the substitution mutation at a position functionally equivalent to Val485 is a mutation to a non-polar, hydrophobic, or uncharged amino acid, for example Gly. The third substitution mutation is at a position functionally equivalent to Ala408 in Pol 1901, where the substitution mutation at a position functionally equivalent to Ala408 is a mutation to a polar or uncharged amino acid, for example Ser. In one embodiment, the substitution mutation at a position functionally equivalent to Phe405 is a mutation to Met, the substitution mutation at a position functionally equivalent to Val485 is a mutation to Gly, and the substitution mutation at a position functionally equivalent to Ala408 is a mutation to Ser.

In one embodiment, an altered polymerase includes at least four substitution mutations. A first substitution mutation is at a position functionally equivalent to Phe405 in Pol 1901 (SEQ ID NO:1), where the substitution mutation at a position functionally equivalent to Phe405 is a mutation to a non-polar amino acid, for example Met. The second substitution mutation is at a position functionally equivalent to Val485 in Pol 1901, where the substitution mutation at a position functionally equivalent to Val485 is a mutation to a non-polar, hydrophobic, or uncharged amino acid, for example Gly. The third substitution mutation is at a position functionally equivalent to Ala408 in Pol 1901, where the substitution mutation at a position functionally equivalent to Ala408 is a mutation to a polar or uncharged amino acid, for example Ser. The fourth substitution mutation is at a position functionally equivalent to Ile410 in Pol 1901, where the substitution mutation at a position functionally equivalent to Ile410 is a mutation to a non-polar or hydrophobic amino acid, for example Val.

In one embodiment, an altered polymerase includes at least four substitution mutations. A first substitution mutation is at a position functionally equivalent to Leu403 in Pol 1901 (SEQ ID NO:1), where the substitution mutation at a position functionally equivalent to Leu403 is a mutation to a non-polar amino acid, for example Met. The second substitution mutation is at a position functionally equivalent to Ala408 in Pol 1901, where the substitution mutation at a position functionally equivalent to Ala408 is a mutation to a polar or uncharged amino acid, for example Ser. The third substitution mutation is at a position functionally equivalent to Ile410 in Pol 1901, where the substitution mutation at a position functionally equivalent to Ile410 is a mutation to a non-polar or hydrophobic amino acid, for example Val. The fourth substitution mutation is at a position functionally equivalent to Gly497 in Pol 1901 (SEQ ID NO:1), where the substitution mutation at a position functionally equivalent to Gly497 is a mutation to a non-polar amino acid, for example Met. In one embodiment, the substitution mutation at a position functionally equivalent to Leu403 is a mutation to Met, the substitution mutation at a position functionally equivalent to Ala408 is a mutation to Ser, the substitution mutation at a position functionally equivalent to Ile410 is a mutation to Val, and the substitution mutation at a position functionally equivalent to Gly497 is a mutation to Met.

In one embodiment, an altered polymerase includes at least five substitution mutations. A first substitution mutation is at a position functionally equivalent to Phe405 in Pol 1901 (SEQ ID NO:1), where the substitution mutation at a position functionally equivalent to Phe405 is a mutation to a non-polar or hydrophobic amino acid, for example Ile. The second substitution mutation is at a position functionally equivalent to Ile410 in Pol 1901, where the substitution mutation at a position functionally equivalent to Ile410 is a mutation to a non-polar or hydrophobic amino acid, for example Pro. The third substitution mutation is at a position functionally equivalent to Ile412 in Pol 1901, where the substitution mutation at a position functionally equivalent to Ile412 is a mutation to a non-polar amino acid, for example Met. The fourth substitution mutation is at a position functionally equivalent to Thr514 in Pol 1901 (SEQ ID NO:1), where the substitution mutation at a position functionally equivalent to Thr514 is a mutation to a non-polar or hydrophobic amino acid, for example Ala. The fifth substitution mutation is at a position functionally equivalent to Ile521 in Pol 1901 (SEQ ID NO:1), where the substitution mutation at a position functionally equivalent to Ile521 is a mutation to a non-polar or hydrophobic amino acid, for example Ala. In one embodiment, the substitution mutation at a position functionally equivalent to Phe405 is a mutation to Ile, the substitution mutation at a position functionally equivalent to Ile410 is a mutation to Pro, the substitution mutation at a position functionally equivalent to Ile412 is a mutation to Met, the substitution mutation at a position functionally equivalent to Thr514 is a mutation to Ala, and the substitution mutation at a position functionally equivalent to Ile521 is a mutation to Ala.

In one embodiment, an altered polymerase includes at least four substitution mutations. A first substitution mutation is at a position functionally equivalent to Phe405 in Pol 1901 (SEQ ID NO:1), where the substitution mutation at a position functionally equivalent to Phe405 is a mutation to a non-polar or hydrophobic amino acid, for example Leu. The second substitution mutation is at a position functionally equivalent to Ala408 in Pol 1901, where the substitution mutation at a position functionally equivalent to Ala408 is a mutation to a polar or uncharged amino acid, for example Ser. The third substitution mutation is at a position functionally equivalent to Ile410 in Pol 1901, where the substitution mutation at a position functionally equivalent to Ile410 is a mutation to a non-polar or hydrophobic amino acid, for example Val. The fourth substitution mutation is at a position functionally equivalent to Ile412 in Pol 1901, where the substitution mutation at a position functionally equivalent to Ile412 is a mutation to a polar or uncharged amino acid, for example Thr. The fifth substitution mutation is at a position functionally equivalent to Thr514 in Pol 1901 (SEQ ID NO:1), where the substitution mutation at a position functionally equivalent to Thr514 is a mutation to a non-polar or hydrophobic amino acid, for example Ala. The sixth substitution mutation is at a position functionally equivalent to Ile521 in Pol 1901 (SEQ ID NO:1), where the substitution mutation at a position functionally equivalent to Ile521 is a mutation to a non-polar or hydrophobic amino acid, for example Ala. In one embodiment, the substitution mutation at a position functionally equivalent to Phe405 is a mutation to Leu, the substitution mutation at a position functionally equivalent to Ala408 is a mutation to Ser, the substitution mutation at a position functionally equivalent to Ile410 is a mutation to Val, the substitution mutation at a position functionally equivalent to Ile412 is a mutation to Thr, the substitution mutation at a position functionally equivalent to Thr514 is a mutation to Ala, and the substitution mutation at a position functionally equivalent to Ile521 is a mutation to Ala.

In one or more embodiments, an altered family B type polymerase of the present disclosure can include additional substitutions mutations. For instance, a polymerase described herein with one or more substitution mutations can also include one or more substitution mutation at a position functionally equivalent to Lys349, Ala281, Trp397, or Gly633 in Pol 1901.

In one embodiment, an altered family B polymerase optionally includes a substitution mutation at a position functionally equivalent to Lys349 in Pol 1901 (SEQ ID NO:1). In one embodiment, the substitution mutation at a position functionally equivalent to Lys349 is a mutation to a polar or uncharged amino acid, for example Asn or Ser.

In one embodiment, an altered family B polymerase optionally includes a substitution mutation at a position functionally equivalent to Ala281 in Pol 1901 (SEQ ID NO:1). In one embodiment, the substitution mutation at a position functionally equivalent to Ala281 is a mutation to a non-polar or hydrophobic amino acid, for example Gly or Phe. In one embodiment, the substitution mutation at a position functionally equivalent to Ala281 is a mutation to an uncharged amino acid, for example Gly.

In one embodiment, an altered family B polymerase optionally includes a substitution mutation at a position functionally equivalent to Phe283 in Pol 1901 (SEQ ID NO:1). In one embodiment, the substitution mutation at a position functionally equivalent to Phe283 is a mutation to a polar or uncharged amino acid, for example Ser.

In one embodiment, an altered family B polymerase optionally includes a substitution mutation at a position functionally equivalent to Trp397 in Pol 1901 (SEQ ID NO:1). In one embodiment, the substitution mutation at a position functionally equivalent to Trp397 is a mutation to a polar or uncharged amino acid, for example Cys. In one embodiment, the substitution mutation at a position functionally equivalent to Trp397 is a mutation to a non-polar or hydrophobic amino acid, for example Phe.

In one embodiment, an altered family B polymerase optionally includes a substitution mutation at a position functionally equivalent to Gly633 in Pol 1901 (SEQ ID NO:1). In one embodiment, the substitution mutation at a position functionally equivalent to Gly633 is a mutation to a polar or uncharged amino acid, for example Thr.

Specific examples of altered polymerases include the polymerases disclosed at SEQ ID NOs:2-32.

An altered polymerase described herein can include additional mutations that are known to affect polymerase activity. On such substitution mutation is at a position functionally equivalent to Arg713 in Pol 1901 (SEQ ID NO:1). Any of a variety of substitution mutations at one or more of positions known to result in reduced exonuclease activity can be made, as is known in the art and exemplified by U.S. Pat. No. 8,623,628. In one embodiment, the substitution mutation at position Arg713 is a mutation to a non-polar, hydrophobic, or uncharged amino acid, for example Gly, Met, or Ala.

In one embodiment, an altered polymerase includes a substitution mutation at a position functionally equivalent to Arg743 or Lys705, or a combination thereof, in Pol 1901 (SEQ ID NO:1), as is known in the art and exemplified by the disclosure of U.S. Pat. No. 8,623,628. In one embodiment, the substitution mutation at position Arg743 or Lys705 is a mutation to a non-polar or hydrophobic amino acid, for example Ala.

Second generation ffNs

A polymerase of the present disclosure can be used with modified nucleotides. A modified nucleotide typically includes a modification at the 3′-OH of the nucleotide sugar moiety, a detectable label attached to the base via a cleavable linker, or both a 3′-OH modification and the detectable label attached to the base via a cleavable linker.

In one or more embodiments, a second generation ffN described herein includes or has the structure of Formula (I):

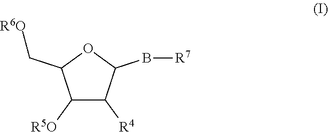

wherein B is the nucleobase; R4 is OH; R5 is H, or a 3′-OH blocking group; R6 is H, monophosphate, diphosphate, triphosphate, thiophosphate, a phosphate ester analog, a reactive phosphorous containing group, or a hydroxy protecting group; R7 is H or L1-L-L2-Label (U.S. Published Patent Application 2021/0403500).

The nucleobase can be purine or a pyrimidine. Examples of pyrimidines include cytosine (C) and thymine (T), 5,6-dihydrouracil and 5-alkylcytosine (e.g., 5-methylcytosine). Examples of purines include adenine (A) and guanine (G), and optionally substituted purine bases such as deazapurine, adenine, 7-deaza adenine, guanine, 7-deaza guanine, hypoxanthine, xanthine, alloxanthine, 7-alkylguanine (e.g. ,7-methylguanine), theobromine, caffeine, uric acid and isoguanine.

Label is a detectable label. Useful detectable labels are known in the art and include, but are not limited to, a fluorophore such as a fluorescent dye.

L is a cleavable linker

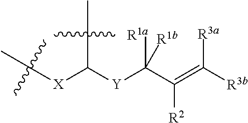

and each of L1 and L2 is independently an optionally present linker moiety.

In some embodiments of the cleavable linker L, each of X and Y is O. In some other embodiments, X is S and Y is O, or X is O and Y is S. In some embodiments each of R1a, R1b, R2 R3a, and R3b is H. In other embodiments, at least one of R1a, R1b, R2, R3a and R3b is halogen (e.g., fluoro, chloro) or unsubstituted C1-C6 alkyl (e.g., methyl, ethyl, isopropyl, isobutyl, or t-butyl). In some such instances, each of R1a and R1b is H and at least one of R2, R3a, and R3b is unsubstituted C1-C6 alkyl or halogen (for example, R2 is unsubstituted C1-C6 alkyl and each of R3a and R3b is H; or R2 is H and one or both of R3a and R3b is halogen or unsubstituted C1-C6 alkyl). In one embodiment, the cleavable linker L includes

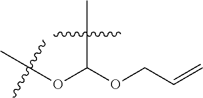 [SMILES: *OC(*)OCC=C]

(the “AOL” linker moiety).

The squiggly lines refer to the attachment of the cleavable linker L to the optional L1 and L2 or, in the absence of L1 and/or L2, the attachment of the cleavable linker L to the nucleobase and/or to the detectable label. In one or more embodiments, the attachment of the optional L1 or L2 or, in the absence of L1 and/or L2, the attachment of the cleavable linker L to the nucleobase is at the C5 position of a pyrimidine base or the C7 position of a purine base.In one or more embodiments, L1-L-L2 is selected from

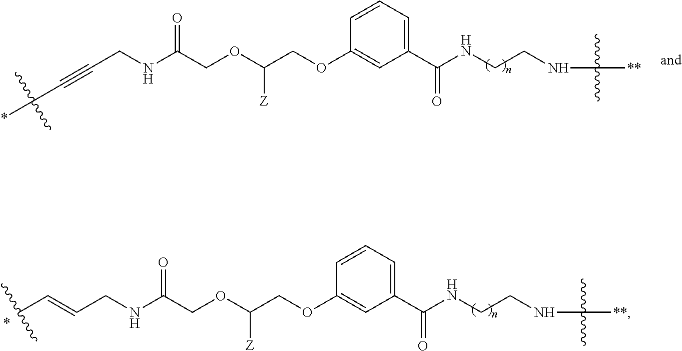

wherein Z is —O—CH₂—CH═CH₂; n is an integer of 1, 2, 3, 4 or 5; * indicates the attachment point of the cleavable linker to the nucleobase; and ** indicates the attachment point of the cleavable linker to the detectable label.

Useful modifications at the 3′-OH of the nucleotide sugar moiety are known in the art, such as a 3′—O—azidomethyl blocking group —CH₂N₃. In one or more embodiments, a polymerase disclosed herein is used with modified nucleotides having a 3′-OH acetal blocking group or a 3′-OH thiocarbamate blocking group (U.S. Pat. No. 11,293,061; U.S. application Ser. No. 17/748,498).

Examples of 3′-hydroxy acetal blocking groups attached to nucleotides having a deoxyribose with the removable 3′-OH blocking group include those forming a structure

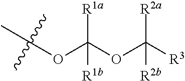

covalently attached to the 3′-carbon atom, where:

each R^{1a} and R^{1b} is independently H, C₁-C₆ alkyl, C₁-C₆ haloalkyl, C₁-C₆ alkoxy, C₁-C₆ haloalkoxy, cyano, halogen, optionally substituted phenyl, or optionally substituted aralkyl; each R^{2a} and R^{2b} is independently H, C₁-C₆ alkyl, C₁-C₆ haloalkyl, cyano, or halogen; alternatively R1a and R2a together with the atoms to which they are attached form an optionally substituted five to eight membered heterocyclyl group;

R³ is H, optionally substituted C₂-C₆ alkenyl, optionally substituted C₃-C₇ cycloalkenyl, optionally substituted C₂-C₆ alkynyl, or optionally substituted (C₁-C₆ alkylene)Si(R⁴)₃; and each R⁴ is independently H, C₁-C₆ alkyl, or optionally substituted C₆-C₁₀ aryl; provided that when each R^{1a}, R^{1b}, R^{2a} and R^{2b} is H, then R³ is not H (all technical and scientific terms regarding 3′-hydroxy acetal blocking groups are defined in U.S. Pat. No. 11,293,061). In one or more embodiments, the 3′-hydroxy acetal blocking group has the following structure

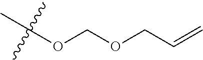 [SMILES: *OCOCC=C]

(the “AOM” 3′ blocking group).

The squiggly line refers to the attachment of the oxygen to the 3′ carbon of the nucleotide sugar.

Examples of 3′-hydroxy thiocarbamate blocking groups attached to nucleotides having a deoxyribose with the removable 3′-OH blocking group include those forming a structure

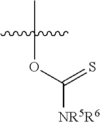

covalently attached to the 3′-carbon atom, where:

each of R⁵ and R⁶ is independently H, C₁-C₆ alkyl, C₂-C₆ alkenyl, C₂-C₆ alkynyl, C₁-C₆ haloalkyl, C₂-C₈ alkoxyalkyl, optionally substituted -(CH₂)ₘ-phenyl, optionally substituted —(CH₂)ₙ-(5 or 6 membered heteroaryl), optionally substituted —(CH₂)ₖ— C₃-C₇ carbocyclyl, or optionally substituted —(CH₂)ₚ-(3 to 7 membered heterocyclyl); alternatively, R⁵ and R⁶ together with the atoms to which they are attached form an optionally substituted five to seven membered heterocycyl;

each of —(CH₂)ₘ—, —(CH₂)ₙ—, —(CH₂)ₖ— and —(CH₂)ₚ— is optionally substituted; and each of m, n, k, and p is independently 0, 1, 2, 3, or 4 (all technical and scientific terms regarding 3′-hydroxy thiocarbamate blocking groups are defined in U.S. Pat. No. 11,293,061).

Non-limiting examples of second generation ffNs useful with an altered polymerase of the present disclosure include, but are not limited to, those with Formula (Ia), (Ia′), (Ib), (Ic), (Ic′), or (Id):

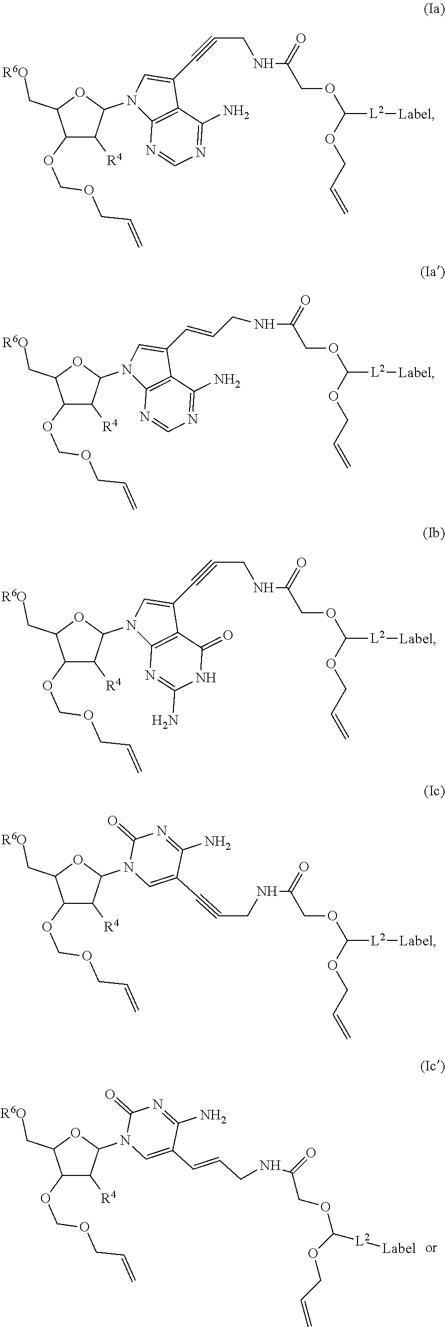

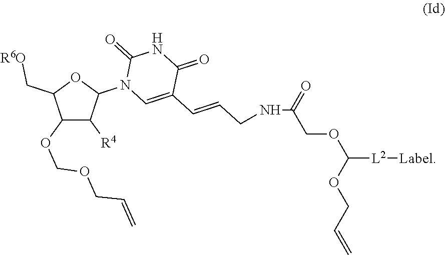

where R⁴ is OH, and where R⁶ is H, monophosphate, diphosphate, triphosphate,

thiophosphate, a phosphate ester analog, a reactive phosphorous containing group, or a hydroxy protecting group.

The methods of the present disclosure include the synthesis of a polynucleotide using second generation ffNs (U.S. Published Patent Application 2021/0403500; U.S. application Ser. No. 17/748,498). An example of a polynucleotide including a nucleotide of Formula (Ia') includes the following structure:

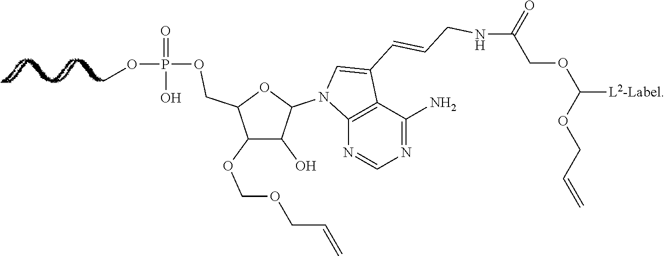

Non-limiting examples of label conjugates, such as fluorescent dye conjugates, include those with the following structures:

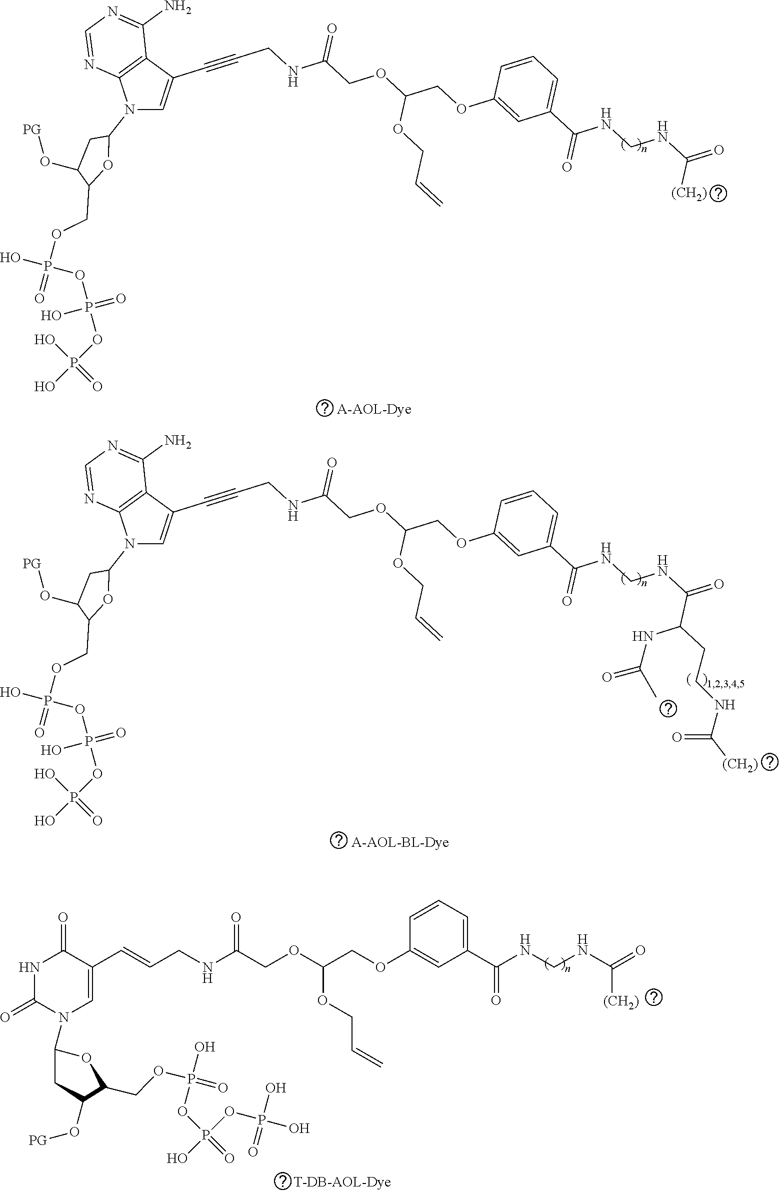

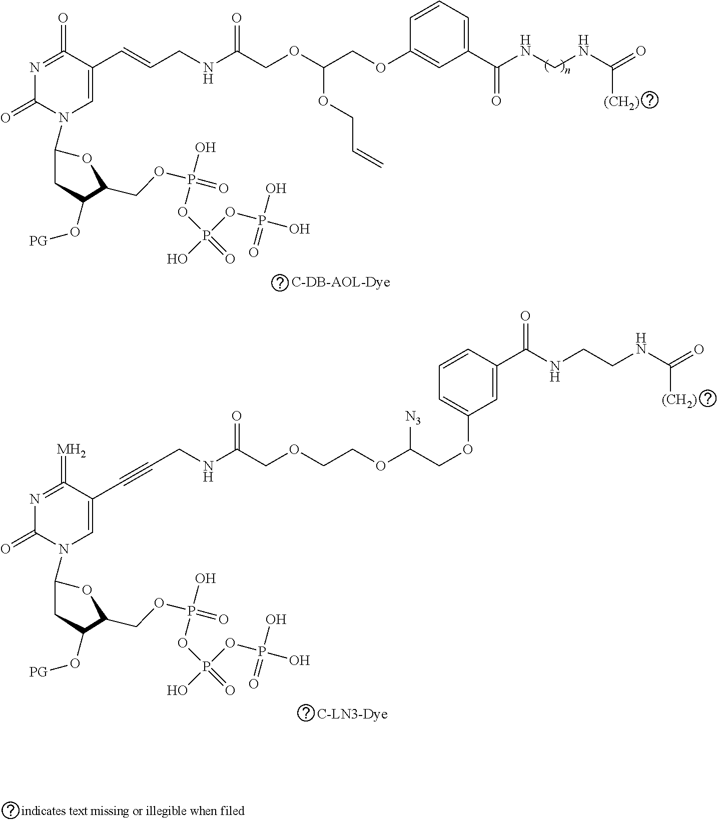

wherein PG stands for the 3′-OH blocking groups described in U.S. Published Patent Application 2021/0403500; n is an integer of 1, 2, 3, 4, 5, 6, 7, 8, 9, or 10; and k is 0, 1, 2, 3, 4, or 5. In one embodiment, —O—PG is AOM. In another embodiment, —O—PG is —O— azidomethyl. In one embodiment, n is 5.

### Mutating Polymerases

Various types of mutagenesis are optionally used in the present disclosure, e.g., to modify polymerases to produce variants, e.g., in accordance with polymerase models and model predictions as discussed herein, or using random or semi-random mutational approaches. In general, any available mutagenesis procedure can be used for making polymerase mutants. Such mutagenesis procedures optionally include selection of mutant nucleic acids and polypeptides for one or more activity of interest (e.g., lower error rate, lower phasing rate, or increased incorporation rate for a second generation ffN). Procedures that can be used include, but are not limited to: site-directed point mutagenesis, random point mutagenesis, in vitro or in vivo homologous recombination (DNA shuffling and combinatorial overlap PCR), mutagenesis using uracil containing templates, oligonucleotide-directed mutagenesis, phosphorothioate-modified DNA mutagenesis, mutagenesis using gapped duplex DNA, point mismatch repair, mutagenesis using repair-deficient host strains, restriction-selection and restriction-purification, deletion mutagenesis, mutagenesis by total gene synthesis, degenerate PCR, double-strand break repair, and many others known to persons of skill. The starting polymerase for mutation can be any of those reference polymerases noted herein, including available polymerase mutants such as those identified in, for instance, U.S. Pat. Nos. 8,460,910, 8,623,628, 10,421,996, 9,765,309, 9,677,057, 11,104,888, 11,001,816.

Optionally, mutagenesis can be guided by known information from a naturally occurring polymerase molecule, or of a known altered or mutated polymerase (e.g., using an existing mutant polymerase), e.g., sequence, sequence comparisons, physical properties, crystal structure and/or the like as discussed above. However, in another class of embodiments, modification can be essentially random (e.g., as in classical or “family” DNA shuffling, see, e.g., Crameri et al. (1998) “DNA shuffling of a family of genes from diverse species accelerates directed evolution” Nature 391:288-291).

Additional information on mutation formats is found in: Sambrook et al., Molecular Cloning—A Laboratory Manual (3rd Ed.), Vol. 1-3, Cold Spring Harbor Laboratory, Cold Spring Harbor, N.Y., 2000 (“Sambrook”); Current Protocols in Molecular Biology, F. M. Ausubel et al., eds., Current Protocols, a joint venture between Greene Publishing Associates, Inc. and John Wiley & Sons, Inc., (supplemented through 2011) (“Ausubel”)) and PCR Protocols A Guide to Methods and Applications (Innis et al. eds) Academic Press Inc. San Diego, Calif. (1990) (“Innis”). The following publications and references cited within provide additional detail on mutation formats: Arnold, Protein engineering for unusual environments, Current Opinion in Biotechnology 4:450-455 (1993); Bass et al., Mutant Trp repressors with new DNA-binding specificities, Science 242:240-245 (1988); Bordo and Argos (1991) Suggestions for “Safe” Residue Substitutions in Site-directed Mutagenesis 217:721-729; Botstein & Shortle, Strategies and applications of in vitro mutagenesis, Science 229:1193-1201 (1985); Carter et al., Improved oligonucleotide site-directed mutagenesis using M13 vectors, Nucl. Acids Res. 13: 4431-4443 (1985); Carter, Site-directed mutagenesis, Biochem. J. 237:1-7 (1986); Carter, Improved oligonucleotide-directed mutagenesis using M13 vectors, Methods in Enzymol. 154: 382-403 (1987); Dale et al., Oligonucleotide-directed random mutagenesis using the phosphorothioate method, Methods Mol. Biol. 57:369-374 (1996); Eghtedarzadeh & Henikoff, Use of oligonucleotides to generate large deletions, Nucl. Acids Res. 14: 5115 (1986); Fritz et al., Oligonucleotide-directed construction of mutations: a gapped duplex DNA procedure without enzymatic reactions in vitro, Nucl. Acids Res. 16: 6987-6999 (1988); Grundstrom et al., Oligonucleotide-directed mutagenesis by microscale ‘shot-gun’ gene synthesis, Nucl. Acids Res. 13: 3305-3316 (1985); Hayes (2002) Combining Computational and Experimental Screening for rapid Optimization of Protein Properties PNAS 99(25) 15926-15931; Kunkel, The efficiency of oligonucleotide directed mutagenesis, in Nucleic Acids & Molecular Biology (Eckstein, F. and Lilley, D. M. J. eds., Springer Verlag, Berlin)) (1987); Kunkel, Rapid and efficient site-specific mutagenesis without phenotypic selection, Proc. Natl. Acad. Sci. USA 82:488-492 (1985); Kunkel et al., Rapid and efficient site-specific mutagenesis without phenotypic selection, Methods in Enzymol. 154, 367-382 (1987); Kramer et al., The gapped duplex DNA approach to oligonucleotide-directed mutation construction, Nucl. Acids Res. 12: 9441-9456 (1984); Kramer & Fritz Oligonucleotide-directed construction of mutations via gapped duplex DNA, Methods in Enzymol. 154:350-367 (1987); Kramer et al., Point Mismatch Repair, Cell 38:879-887 (1984); Kramer et al., Improved enzymatic in vitro reactions in the gapped duplex DNA approach to oligonucleotide-directed construction of mutations, Nucl. Acids Res. 16: 7207 (1988); Ling et al., Approaches to DNA mutagenesis: an overview, Anal Biochem. 254(2): 157-178 (1997); Lorimer and Pastan Nucleic Acids Res. 23, 3067-8 (1995); Mandecki, Oligonucleotide-directed double-strand break repair in plasmids of Escherichia coli: a method for site-specific mutagenesis, Proc. Natl. Acad. Sci. USA, 83:7177-7181(1986); Nakamaye & Eckstein, Inhibition of restriction endonuclease Nci I cleavage by phosphorothioate groups and its application to oligonucleotide-directed mutagenesis, Nucl. Acids Res. 14: 9679-9698 (1986); Nambiar et al., Total synthesis and cloning of a gene coding for the ribonuclease S protein, Science 223: 1299-1301(1984); Sakamar and Khorana, Total synthesis and expression of a gene for the a-subunit of bovine rod outer segment guanine nucleotide-binding protein (transducin), Nucl. Acids Res. 14: 6361-6372 (1988); Sayers et al., Y-T Exonucleases in phosphorothioate-based oligonucleotide-directed mutagenesis, Nucl. Acids Res. 16:791-802 (1988); Sayers et al., Strand specific cleavage of phosphorothioate-containing DNA by reaction with restriction endonucleases in the presence of ethidium bromide, (1988) Nucl. Acids Res. 16: 803-814; Sieber, et al., Nature Biotechnology, 19:456-460 (2001); Smith, In vitro mutagenesis, Ann. Rev. Genet. 19:423-462 (1985); Methods in Enzymol. 100: 468-500 (1983); Methods in Enzymol. 154: 329-350 (1987); Stemmer, Nature 370, 389-91(1994); Taylor et al., The use of phosphorothioate-modified DNA in restriction enzyme reactions to prepare nicked DNA, Nucl. Acids Res. 13: 8749-8764 (1985); Taylor et al., The rapid generation of oligonucleotide-directed mutations at high frequency using phosphorothioate-modified DNA, Nucl. Acids Res. 13: 8765-8787 (1985); Wells et al., Importance of hydrogen-bond formation in stabilizing the transition state of subtilisin, Phil. Trans. R. Soc. Lond. A 317: 415-423 (1986); Wells et al., Cassette mutagenesis: an efficient method for generation of multiple mutations at defined sites, Gene 34:315-323 (1985); Zoller & Smith, Oligonucleotide-directed mutagenesis using M 13-derived vectors: an efficient and general procedure for the production of point mutations in any DNA fragment, Nucleic Acids Res. 10:6487-6500 (1982); Zoller & Smith, Oligonucleotide-directed mutagenesis of DNA fragments cloned into M13 vectors, Methods in Enzymol. 100:468-500 (1983); Zoller & Smith, Oligonucleotide-directed mutagenesis: a simple method using two oligonucleotide primers and a single-stranded DNA template, Methods in Enzymol. 154:329-350 (1987); Clackson et al. (1991) “Making antibody fragments using phage display libraries” Nature 352:624-628; Gibbs et al. (2001) “Degenerate oligonucleotide gene shuffling (DOGS): a method for enhancing the frequency of recombination with family shuffling” Gene 271:13-20; and Hiraga and Arnold (2003) “General method for sequence-independent site-directed chimeragenesis: J. Mol. Biol. 330:287-296. Additional details on many of the above methods can be found in Methods in Enzymology Volume 154, which also describes useful controls for trouble-shooting problems with various mutagenesis methods.

### Making and Isolating Recombinant Polymerases

Generally, nucleic acids encoding a polymerase as presented herein can be made by cloning, recombination, in vitro synthesis, in vitro amplification and/or other available methods, or obtained from a commercial vendor. A variety of recombinant methods can be used for expressing an expression vector that encodes a polymerase of the present disclosure. Methods for making recombinant nucleic acids, expression and isolation of expressed products are well known and described in the art. A number of exemplary mutations and combinations of mutations, as well as strategies for design of desirable mutations, are described herein. Methods for making and selecting mutations in the active site of polymerases, including for modifying steric features in or near the active site to permit improved access by modified nucleotides are found herein and, e.g., in WO 2007/076057 and WO 2008/051530.

Additional useful references for mutation, recombinant and in vitro nucleic acid manipulation methods (including cloning, expression, PCR, and the like) include Berger and Kimmel, Guide to Molecular Cloning Techniques, Methods in Enzymology volume 152 Academic Press, Inc., San Diego, Calif. (Berger); Kaufman et al. (2003) Handbook of Molecular and Cellular Methods in Biology and Medicine Second Edition Ceske (ed) CRC Press (Kaufman); The Nucleic Acid Protocols Handbook Ralph Rapley (ed) (2000) Cold Spring Harbor, Humana Press Inc (Rapley); Chen et al. (ed) PCR Cloning Protocols, Second Edition (Methods in Molecular Biology, volume 192) Humana Press; and in Viljoen et al. (2005) Molecular Diagnostic PCR Handbook Springer, ISBN 1402034032.

In addition, many kits are commercially available for the purification of plasmids or other relevant nucleic acids from cells, (see, e.g., StrataClean™, from Stratagene; and QIAprep™ from Qiagen). Any isolated and/or purified nucleic acid can be further manipulated to produce other nucleic acids, used to transfect cells, incorporated into related vectors to infect organisms for expression, and/or the like. Typical cloning vectors contain transcription and translation terminators, transcription and translation initiation sequences, and promoters useful for regulation of the expression of the particular target nucleic acid. The vectors optionally comprise generic expression cassettes containing at least one independent terminator sequence, sequences permitting replication of the cassette in eukaryotes, or prokaryotes, or both, (e.g., shuttle vectors) and selection markers for both prokaryotic and eukaryotic systems. Vectors are suitable for replication and integration in prokaryotes, eukaryotes, or both.

Other useful references, e.g. for cell isolation and culture (e.g., for subsequent nucleic acid isolation) include Freshney (1994) Culture of Animal Cells, a Manual of Basic Technique, third edition, Wiley-Liss, New York and the references cited therein; Payne et al. (1992) Plant Cell and Tissue Culture in Liquid Systems John Wiley & Sons, Inc. New York, N.Y.; Gamborg and Phillips (eds) (1995) Plant Cell, Tissue and Organ Culture; Fundamental Methods Springer Lab Manual, Springer-Verlag (Berlin Heidelberg New York); and Atlas and Parks (eds) The Handbook of Microbiological Media (1993) CRC Press, Boca Raton, Fla. Construction of vectors containing a nucleic acid encoding an altered polymerase described herein employs standard ligation techniques known in the art. See, e.g., Sambrook et al, Molecular Cloning: A Laboratory Manual., Cold Spring Harbor Laboratory Press (1989) or Ausubel, R. M., ed. Current Protocols in Molecular Biology (1994).

The present disclosure also includes nucleic acids encoding the altered polymerases disclosed herein. A particular amino acid can be encoded by multiple codons, and certain translation systems (e.g., prokaryotic or eukaryotic cells) often exhibit codon bias, e.g., different organisms often prefer one of the several synonymous codons that encode the same amino acid. As such, nucleic acids presented herein are optionally “codon optimized,” meaning that the nucleic acids are synthesized to include codons that are preferred by the particular translation system being employed to express the polymerase. For example, when it is desirable to express the polymerase in a bacterial cell (or even a particular strain of bacteria), the nucleic acid can be synthesized to include codons most frequently found in the genome of that bacterial cell, for efficient expression of the polymerase. A similar strategy can be employed when it is desirable to express the polymerase in a eukaryotic cell, e.g., the nucleic acid can include codons preferred by that eukaryotic cell.

A variety of protein isolation and detection methods are known and can be used to isolate polymerases, e.g., from recombinant cultures of cells expressing the recombinant polymerases presented herein. A variety of protein isolation and detection methods are well known in the art, including, e.g., those set forth in R. Scopes, Protein Purification, Springer-Verlag, N.Y. (1982); Deutscher, Methods in Enzymology Vol. 182: Guide to Protein Purification, Academic Press, Inc. N.Y. (1990); Sandana (1997) Bioseparation of Proteins, Academic Press, Inc .; Bollag et al. (1996) Protein Methods, 2nd Edition Wiley-Liss, NY; Walker (1996) The Protein Protocols Handbook Humana Press, NJ, Harris and Angal (1990) Protein Purification Applications: A Practical Approach IRL Press at Oxford, Oxford, England; Harris and Angal Protein Purification Methods: A Practical Approach IRL Press at Oxford, Oxford, England; Scopes (1993) Protein Purification: Principles and Practice 3rd Edition Springer Verlag, NY; Janson and Ryden (1998) Protein Purification: Principles, High Resolution Methods and Applications, Second Edition Wiley-VCH, NY; and Walker (1998) Protein Protocols on CD-ROM Humana Press, NJ; and the references cited therein. Additional details regarding protein purification and detection methods can be found in Satinder Ahuja ed., Handbook of Bioseparations, Academic Press (2000).

### Methods of Use

The altered polymerases presented herein can be used in a sequencing procedure, such as a sequencing-by-synthesis (SBS) technique. Briefly, SBS can be initiated by contacting the target nucleic acids with one or more nucleotides (e.g., labelled, synthetic, modified, or a combination thereof), DNA polymerase, etc. Those features where a primer is extended using the target nucleic acid as template will incorporate a labeled nucleotide that can be detected. Use of an altered polymerase described herein can result in lower error rate, lower phasing rate, lower pre-phasing, or increased incorporation rate (i.e., reduced incorporation time) in a sequencing run. In one or more embodiments, the labeled nucleotides can be modified, e.g., further include a reversible termination property that terminates further primer extension once a nucleotide has been added to a primer. For example, a modified nucleotide having a reversible terminator moiety can be added to a primer such that subsequent extension cannot occur until a deblocking agent is delivered to remove the moiety. In one or more embodiments, the reversible terminator moiety includes an 3′-OH acetal blocking group or a 3′-OH thiocarbamate blocking group. Thus, for embodiments that use reversible termination, a deblocking reagent can be delivered to the flow cell (before or after detection occurs). Washes can be carried out between the various delivery steps. The cycle can then be repeated n times to extend the primer by n nucleotides, thereby detecting a sequence of length n. Exemplary SBS procedures, fluidic systems, and detection platforms that can be readily adapted for use with an array produced by the methods of the present disclosure are described, for example, in Bentley et al., Nature 456:53-59 (2008); WO 04/018497; WO 91/06678; WO 07/123744; U.S. Pat. Nos. 7,057,026, 7,329,492, 7,211,414, 7,315,019, 7,405,281, and 8,343,746.

Other sequencing procedures that use cyclic reactions can be used, such as pyrosequencing. Pyrosequencing detects the release of inorganic pyrophosphate (PPi) as particular nucleotides are incorporated into a nascent nucleic acid strand (Ronaghi, et al., Analytical Biochemistry 242(1), 84-9 (1996); Ronaghi, Genome Res. 11(1), 3-11 (2001); Ronaghi et al. Science 281(5375), 363 (1998); U.S. Pat. Nos. 6,210,891; 6,258,568 and 6,274,320). In pyrosequencing, released PPi can be detected by being converted to adenosine triphosphate (ATP) by ATP sulfurylase, and the resulting ATP can be detected via luciferase-produced photons. Thus, the sequencing reaction can be monitored via a luminescence detection system. Excitation radiation sources used for fluorescence-based detection systems are not necessary for pyrosequencing procedures. Useful fluidic systems, detectors and procedures that can be used for application of pyrosequencing to arrays of the present disclosure are described, for example, in WO 2012/058096, US Pat. App. Pub. No. 2005/0191698 A1, U.S. Pat. Nos. 7,595,883 and 7,244,559.

Some embodiments can use methods involving the real-time monitoring of DNA polymerase activity. For example, nucleotide incorporations can be detected through fluorescence resonance energy transfer (FRET) interactions between a fluorophore-bearing polymerase and γ-phosphate-labeled nucleotides, or with zeromode waveguides. Techniques and reagents for FRET-based sequencing are described, for example, in Levene et al. Science 299, 682-686 (2003); Lundquist et al. Opt. Lett. 33, 1026-1028 (2008); Korlach et al. Proc. Natl. Acad. Sci. USA 105, 1176-1181 (2008).

Some SBS embodiments include detection of a proton released upon incorporation of a nucleotide into an extension product. For example, sequencing based on detection of released protons can use an electrical detector and associated techniques that are commercially available from Ion Torrent (Thermo Fisher Scientific) or sequencing methods and systems described in U.S. Pat. Nos. 8,262,900, 7,948,015, 8,349,167, and US Published Patent Application No. 2010/0137143 A1.

Accordingly, presented herein are methods for incorporating modified nucleotides into DNA including allowing the following components to interact: (i) an altered polymerase described herein, (ii) a DNA template; and (iii) a nucleotide solution. In certain embodiments, the DNA template can be associated with an array, including a clustered array. The DNA template can be double-stranded or single-stranded. In one or more embodiments, the nucleotides are modified nucleotides, such as a second generation ffN described herein. A modified nucleotide typically includes a modification at the 3′-OH of the nucleotide sugar moiety, a detectable label attached to the base via a cleavable linker, or both a 3′-OH modification and the detectable label attached to the base via a cleavable linker.

### Nucleic Acids Encoding Altered Polymerases

The present disclosure also includes nucleic acid molecules encoding the altered polymerases described herein. For any given altered polymerase which is a mutant version of a polymerase for which the amino acid sequence and preferably also the wild type nucleotide sequence encoding the polymerase is known, it is possible to obtain a nucleotide sequence encoding the mutant according to the basic principles of molecular biology. For example, given that the wild type nucleotide sequence encoding 9° N polymerase is known, it is possible to deduce a nucleotide sequence encoding any given mutant version of 9° N having one or more amino acid substitutions using the standard genetic code. Similarly, nucleotide sequences can readily be derived for mutant versions other polymerases such as, for example, Vent® polymerase, Deep Vent® polymerase, Pfu polymerase, KOD polymerase, Pab polymerase, etc. Nucleic acid molecules having the required nucleotide sequence may then be constructed using standard molecular biology techniques known in the art.

In accordance with the embodiments presented herein, a defined nucleic acid includes not only the identical nucleic acid but also any minor base variations including, in particular, substitutions in cases which result in a synonymous codon (a different codon specifying the same amino acid residue) due to the degenerate code in conservative amino acid substitutions. The class of nucleotide sequences encoding an altered polymerase disclosed herein is large but finite, and the nucleotide sequence of each member of the class may be readily determined by reference to the standard genetic code.

The term “nucleic acid sequence” also includes the complementary sequence to any single stranded sequence given regarding base variations, and the corresponding RNA sequences.

The nucleic acid molecules described herein may also, advantageously, be included in a suitable expression vector to express the altered polymerase proteins encoded therefrom in a suitable host. Incorporation of cloned DNA into a suitable expression vector for subsequent transformation of said cell and subsequent selection of the transformed cells is well known to those skilled in the art as provided in Sambrook et al. (1989), Molecular cloning: A Laboratory Manual, Cold Spring Harbor Laboratory.

Such an expression vector includes a vector having a nucleic acid according to the embodiments presented herein operably linked to regulatory sequences, such as promoter regions, that are capable of effecting expression of said DNA fragments. The term “operably linked” refers to a juxtaposition wherein the components described are in a relationship permitting them to function in their intended manner. Such vectors may be transformed into a suitable host cell to provide for the expression of a protein according to the embodiments presented herein.

The nucleic acid molecule may encode a mature protein or a protein having a pro-sequence, including that encoding a leader sequence on the preprotein which is then cleaved by the host cell to form a mature protein. The vectors may be, for example, plasmid, virus or phage vectors provided with an origin of replication, and optionally a promoter for the expression of said nucleotide and optionally a regulator of the promoter. The vectors may contain one or more selectable markers, such as, for example, an antibiotic resistance gene.

Regulatory elements required for expression include promoter sequences to bind RNA polymerase and to direct an appropriate level of transcription initiation and also translation initiation sequences for ribosome binding. For example, a bacterial expression vector may include a promoter such as the lac promoter and for translation initiation the Shine-Dalgarno sequence and the start codon AUG. Similarly, a eukaryotic expression vector may include a heterologous or homologous promoter for RNA polymerase II, a downstream polyadenylation signal, the start codon AUG, and a termination codon for detachment of the ribosome. Such vectors may be obtained commercially or be assembled from the sequences described by methods well known in the art.

Transcription of DNA encoding the polymerase by higher eukaryotes may be optimized by including an enhancer sequence in the vector. Enhancers are cis-acting elements of DNA that act on a promoter to increase the level of transcription. Vectors will also generally include origins of replication in addition to the selectable markers.

### Kits

The present disclosure also provides kits for performing a nucleotide incorporation reaction. The kit includes at least one altered polymerase described herein and a nucleotide solution in a suitable packaging material in an amount sufficient for at least one nucleotide incorporation reaction. Optionally, other reagents such as buffers and solutions needed to use the altered polymerase and nucleotide solution are also included. Examples of other reagents include deblocking reagents, palladium catalysts, palladium scavengers, and the like suitable for use with second generation ffNs (U.S. Published Patent Application 2021/0403500; U.S. application Ser. No. 17/748,498). Instructions for use of the packaged components may be included.

In certain embodiments, the nucleotide solution includes labelled nucleotides. In certain embodiments, the nucleotides are synthetic nucleotides. In certain embodiments, the nucleotides are modified nucleotides, such as a second generation ffN. In certain embodiments, a modified nucleotide has been modified at the 3′ sugar hydroxyl such that the substituent is larger in size than the naturally occurring 3′ hydroxyl group. In certain embodiments, the modified nucleotides include a modified nucleotide molecule that includes a purine or pyrimidine base and a deoxyribose sugar moiety having a removable 3′-OH blocking group covalently attached thereto. In one or more embodiments, the 3′ carbon atom has attached a 3′-OH acetal blocking group or a 3′-OH thiocarbamate blocking group. 3′-OH acetal blocking groups and 3′-OH thiocarbamate blocking groups are described herein. In one or more embodiments, the 3′-hydroxy acetal blocking group has the following structure.

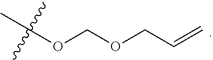 [SMILES: *OCOCC=C]

In certain embodiments, the modified nucleotides are fluorescently labelled to allow their detection. In certain embodiments, the modified nucleotides include a nucleotide having a base attached to a detectable label via a cleavable linker. In certain embodiments, the detectable label includes a fluorescent label. In certain embodiments the cleavable linker has the following structure

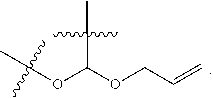 [SMILES: *OC(*)OCC=C]

As used herein, the phrase “packaging material” refers to one or more physical structures used to house the contents of the kit. The packaging material is constructed by known methods, preferably to provide a sterile, contaminant-free environment. The packaging material has a label which indicates that the components can be used for conducting a nucleotide incorporation reaction. In addition, the packaging material may contain instructions indicating how the materials within the kit are employed to practice a nucleotide incorporation reaction. As used herein, the term “package” refers to a solid matrix or material such as glass, plastic, paper, foil, and the like, capable of holding within fixed limits the polypeptides. “Instructions for use” typically include a tangible expression describing the reagent concentration or at least one assay method parameter, such as the relative amounts of reagent and sample to be admixed, maintenance time periods for reagent/sample admixtures, temperature, buffer conditions, and the like.

### Compositions

The present disclosure also provides compositions that include an altered polymerase described herein. The composition can include other components in addition to the altered polymerase. For example, the composition can include a buffer, a nucleotide solution, or a combination thereof. The nucleotide solution can include nucleotides, such as nucleotides that are labelled, synthetic, modified, or a combination thereof. In one embodiment, a composition includes target nucleic acids, such as a library of target nucleic acids. In one embodiment, a composition can include the altered polymerase present with an array, such as a flowcell or a bead.

The invention is defined in the claims. However, below there is provided a non-exhaustive listing of non-limiting exemplary aspects. Any one or more of the features of these aspects may be combined with any one or more features of another example, embodiment, or aspect described herein.

### Exemplary Aspects

Aspect 1 is an altered archaeal Family B DNA polymerase, wherein the altered archaeal Family B DNA polymerase comprises an amino acid substitution mutation at a position functionally equivalent to an amino acid in a reference archaeal Family B DNA polymerase of SEQ ID NO:1, and the altered archaeal Family B DNA polymerase is capable of incorporating a modified nucleotide comprising a 3′-OH acetal blocking group or a 3′-OH thiocarbamate blocking group at (i) a lower error rate, (ii) a lower phasing rate, or both (i) and (ii), compared to SEQ ID NO:1; wherein the amino acid is at position Arg58 and comprises a mutation to Leu; wherein the amino acid is at position Tyr261 and comprises a mutation to Gly; wherein the amino acid is at position Asn269 and comprises a mutation to Gly or Val; wherein the amino acid is at position Phe283 and comprises a mutation to Ile or Lys; wherein the amino acid is at position Pro328 and comprises a mutation to Asp; wherein the amino acid is at position Met329 and comprises a mutation to Thr; wherein the amino acid is at position Gln332 and comprises a mutation to Ser; wherein the amino acid is at position Leu333 and comprises a mutation to His or Ile; wherein the amino acid is at position Ser347 and comprises a mutation to Asp or Arg or Glu or Val or Thr; wherein the amino acid is at position Asn399 and comprises a mutation to Met or Ala; wherein the amino acid is at position Phe405 and comprises a mutation to Met or Lys or Gln; wherein the amino acid is at position Arg406 and comprises a mutation to Met; wherein the amino acid is at position Ile410 and comprises a mutation to Val; wherein the amino acid is at position Ile412 and comprises a mutation to Val; wherein the amino acid is at position Glu458 and comprises a mutation to Gly; wherein the amino acid is at position Glu459 and comprises a mutation to Thr or Asp; wherein the amino acid is at position Gln461 and comprises a mutation to Trp; wherein the amino acid is at position Ala469 and comprises a mutation to Glu; wherein the amino acid is at position Tyr481 and comprises a mutation to Ile; wherein the amino acid is at position Val485 and comprises a mutation to Asn or Gly or Met or Gln or Ser or Thr; wherein the amino acid is at position Ile486 and comprises a mutation to Leu; wherein the amino acid is at position Trp504 and comprises a mutation to Asn; wherein the amino acid is at position Lys507 and comprises a mutation to His or Pro; wherein the amino acid is at position Glu511 and comprises a mutation to Lys or Met or Arg; wherein the amino acid is at position Trp516 and comprises a mutation to Ile or Lys or Met or Gln or Leu; wherein the amino acid is at position Tyr520 and comprises a mutation to Ala; wherein the amino acid is at position Ile521 and comprises a mutation to Thr; wherein the amino acid is at position Met523 and comprises a mutation to Ile or Thr; wherein the amino acid is at position Arg526 and comprises a mutation to Asn; wherein the amino acid is at position Glu527 and comprises a mutation to Ile; wherein the amino acid is at position Leu528 and comprises a mutation to Thr; wherein the amino acid is at position Ile567 and comprises a mutation to Leu; wherein the amino acid is at position Asn568 and comprises a mutation to Gln; wherein the amino acid is at position Leu571 and comprises a mutation to Phe or Ile or Met or Trp; wherein the amino acid is at position Glu576 and comprises a mutation to Leu; wherein the amino acid is at position Glu580 and comprises a mutation to Ile or Lys or Gln or Arg or Val or Met; wherein the amino acid is at position Thr590 and comprises a mutation to Lys; wherein the amino acid is at position Ala595 and comprises a mutation to Met; or wherein the amino acid is at position Ile603 and comprises a mutation to Asp, or a combination thereof.

Aspect 2 is the altered archaeal Family B DNA polymerase of aspect 1, wherein the amino acid is at position Leu333 and comprises a mutation to His; wherein the amino acid is at position Ser347 and comprises a mutation to Asp or Arg; wherein the amino acid is at position Asn399 and comprises a mutation to Met; wherein the amino acid is at position Phe405 and comprises a mutation to Met; wherein the amino acid is at position Tyr481 and comprises a mutation to Ile; wherein the amino acid is at position Val485 and comprises a mutation to Asn; wherein the amino acid is at position Ile486 and comprises a mutation to Lys; wherein the amino acid is at position Trp504 and comprises a mutation to Asn; wherein the amino acid is at position Glu511 and comprises a mutation to Lys or Met; wherein the amino acid is at position Asn568 and comprises a mutation to Gln; or wherein the amino acid is at position Glu580 and comprises a mutation to Ile or Lys or Gln or Arg or Val, or a combination thereof.

Aspect 3 is the altered archaeal Family B DNA polymerase of aspect 1 or 2, wherein the amino acid is at position Asn269 and comprises a mutation to Gly or Val; wherein the amino acid is at position Leu333 and comprises a mutation to Ile; wherein the amino acid is at position Ser347 and comprises a mutation to Glu or Val; wherein the amino acid is at position Asn399 and comprises a mutation to Ala; wherein the amino acid is at position Phe405 and comprises a mutation to Lys or Gln; wherein the amino acid is at position Ile412 and comprises a mutation to Val; wherein the amino acid is at position Glu459 and comprises a mutation to Thr; wherein the amino acid is at position Gln461 and comprises a mutation to Trp; wherein the amino acid is at position Val485 and comprises a mutation to Gly or Met or Gln or Ser or Thr; wherein the amino acid is at position Glu511 and comprises a mutation to Arg; wherein the amino acid is at position Trp516 and comprises a mutation to Ile or Lys or Met or Gln; wherein the amino acid is at position Tyr520 and comprises a mutation to Ala; wherein the amino acid is at position Ile567 and comprises a mutation to Leu; or wherein the amino acid is at position Glu580 and comprises a mutation to Met, or a combination thereof.

Aspect 4 is an altered archaeal Family B DNA polymerase comprising an amino acid substitution mutation at a position functionally equivalent to Glu580 in a reference archaeal Family B DNA polymerase of SEQ ID NO:1, and the altered archaeal Family B DNA polymerase is capable of incorporating a modified nucleotide comprising a 3′-OH acetal blocking group or a 3′-OH thiocarbamate blocking group at (i) a lower error rate, (ii) a lower phasing rate, or both (i) and (ii), compared to SEQ ID NO:1.

Aspect 5 is the altered archaeal Family B DNA polymerase of aspect 4, wherein the substitution mutation at the position functionally equivalent to Glu580 comprises a mutation to a polar amino acid.

Aspect 6 is the altered archaeal Family B DNA polymerase of aspect 4 or 5, wherein the substitution mutation at the position functionally equivalent to Glu580 comprises a mutation to Lys or Arg.

Aspect 7 is the altered archaeal Family B DNA polymerase of any of aspects 4-6, the polymerase further comprising an amino acid substitution mutation at a position functionally equivalent to Phe405 in the reference archaeal Family B DNA polymerase.

Aspect 8 is the altered archaeal Family B DNA polymerase of any of aspects 4-7, wherein the substitution mutation at the position functionally equivalent to Phe405 comprises a mutation to a non-polar or hydrophobic amino acid.

Aspect 9 is the altered archaeal Family B DNA polymerase of any of aspects 4-8, wherein the substitution mutation at the position functionally equivalent to Phe405 comprises a mutation to Met.

Aspect 10 is the altered archaeal Family B DNA polymerase of any of aspects 4-9, wherein the substitution mutation at the position functionally equivalent to Glu580 comprises a mutation to Arg, further comprising a substitution mutation at a position functionally equivalent to Phe405 that comprises a mutation to Met.

Aspect 11 is the altered archaeal Family B DNA polymerase of any of aspects 4-10, the polymerase further comprising an amino acid substitution mutation at a position functionally equivalent to Val485 in the reference archaeal Family B DNA polymerase.

Aspect 12 is the altered archaeal Family B DNA polymerase of any of aspects 4-11, wherein the substitution mutation at the position functionally equivalent to Val485 comprises a mutation to a polar or uncharged amino acid.

Aspect 13 is the altered archaeal Family B DNA polymerase of any of aspects 4-12, wherein the substitution mutation at the position functionally equivalent to Val485 comprises a mutation to Thr.

Aspect 14 is the altered archaeal Family B DNA polymerase of any of aspects 4-13, wherein the substitution mutation at the position functionally equivalent to Glu580 comprises a mutation to Lys, further comprising a substitution mutation at a position functionally equivalent to Val485 that comprises a mutation to Thr.

Aspect 15 is the altered archaeal Family B DNA polymerase of any of aspects 4-14, the polymerase further comprising an amino acid substitution mutation at a position functionally equivalent to Trp516 in the reference archaeal Family B DNA polymerase.

Aspect 16 is the altered archaeal Family B DNA polymerase of any of aspects 4-15, wherein the substitution mutation at the position functionally equivalent to Trp516 comprises a mutation to a non-polar or hydrophobic amino acid.

Aspect 17 is the altered archaeal Family B DNA polymerase of any of aspects 4-16, wherein the substitution mutation at the position functionally equivalent to Trp516 comprises a mutation to Met or to Leu.

Aspect 18 is the altered archaeal Family B DNA polymerase of any of aspects 4-17, the polymerase further comprising an amino acid substitution mutation at a position functionally equivalent to Leu571 in the reference archaeal Family B DNA polymerase.

Aspect 19 is the altered archaeal Family B DNA polymerase of any of aspects 4-18, wherein the substitution mutation at the position functionally equivalent to Leu571 comprises a mutation to a non-polar or hydrophobic amino acid.

Aspect 20 is the altered archaeal Family B DNA polymerase of aspect any of aspects 4-19, wherein the substitution mutation at the position functionally equivalent to Leu571 comprises a mutation to Phe or Met.

Aspect 21 is the altered archaeal Family B DNA polymerase of any of aspects 4-20, wherein the substitution mutation at the position functionally equivalent to Glu580 comprises a mutation to Lys, further comprising a substitution mutation at a position functionally equivalent to Leu571 that comprises a mutation to Phe, or wherein the substitution mutation at the position functionally equivalent to Glu580 comprises a mutation to Gln, further comprising a substitution mutation at a position functionally equivalent to Leu571 that comprises a mutation to Met.

Aspect 22 is the altered archaeal Family B DNA polymerase of any of aspects 4-21, the polymerase further comprising an amino acid substitution mutation at a position functionally equivalent to Ala408, Ile410, or both Ala408 and Ile410, in the reference archaeal Family B DNA polymerase.

Aspect23 is the altered archaeal Family B DNA polymerase of any of aspects 4-22, wherein the substitution mutation at the position functionally equivalent to Ala408 comprises a mutation to a polar or uncharged amino acid.

Aspect 24 is the altered archaeal Family B DNA polymerase of any of aspects 4-23, wherein the substitution mutation at the position functionally equivalent to Ala408 comprises a mutation to Ser.

Aspect 25 is the altered archaeal Family B DNA polymerase of any of aspects 4-24, wherein the substitution mutation at the position functionally equivalent to Ile410 comprises a mutation to a non-polar or hydrophobic amino acid.

Aspect 26 is the altered archaeal Family B DNA polymerase of any of aspects 4-25, wherein the substitution mutation at the position functionally equivalent to Ile410 comprises a mutation to Val.

Aspect 27 is the altered archaeal Family B DNA polymerase of any of aspects 4-26, wherein the substitution mutation at the position functionally equivalent to Glu580 comprises a mutation to Lys, further comprising a substitution mutation at a position functionally equivalent to Trp516 that comprises a mutation to Met or to Leu.

Aspect 28 is the altered archaeal Family B DNA polymerase of any of aspects 4-27, wherein the substitution mutation at the position functionally equivalent to Glu580 comprises a mutation to Lys, further comprising a substitution mutation at a position functionally equivalent to Ala408 that comprises a mutation to Ser and a substitution mutation at a position functionally equivalent to Ile410 that comprises a mutation to Val.

Aspect 29 is the altered archaeal Family B DNA polymerase of any of aspects 4-28, wherein the substitution mutation at the position functionally equivalent to Glu580 comprises a mutation to Lys, further comprising a substitution mutation at a position functionally equivalent to Ala408 that comprises a mutation to Ser.

Aspect 30 is the altered archaeal Family B DNA polymerase of any of aspects 4-29, wherein the substitution mutation at the position functionally equivalent to Glu580 comprises a mutation to Lys, further comprising a substitution mutation at a position functionally equivalent to Phe405 that comprises a mutation to a non-polar or hydrophobic amino acid.

Aspect 31 is the altered archaeal Family B DNA polymerase of any of aspects 4-30, wherein the substitution mutation at the position functionally equivalent to Phe405 comprises a mutation to Met.

Aspect 32 is the altered archaeal Family B DNA polymerase of any of aspects 4-31, wherein the substitution mutation at the position functionally equivalent to Glu580 comprises a mutation to Lys, further comprising a substitution mutation at a position functionally equivalent to Phe405 that comprises a mutation to Met.

Aspect 33 is the altered archaeal Family B DNA polymerase of any of aspects 4-32, wherein the substitution mutation at the position functionally equivalent to Glu580 comprises a mutation to Lys, further comprising a substitution mutation at a position functionally equivalent to Ile410 that comprises a mutation to a non-polar amino acid.

Aspect 34 is the altered archaeal Family B DNA polymerase of any of aspects 4-33, wherein the substitution mutation at the position functionally equivalent to Ile410 comprises a mutation to Val.

Aspect 35 is the altered archaeal Family B DNA polymerase of any of aspects 4-34, wherein the substitution mutation at the position functionally equivalent to Glu580 comprises a mutation to Lys, further comprising a substitution mutation at a position functionally equivalent to Ile410 that comprises a mutation to Val.

Aspect 36 is an altered archaeal Family B DNA polymerase comprising an amino acid substitution mutation at a position functionally equivalent to Phe405 and Val485 in a reference archaeal Family B DNA polymerase of SEQ ID NO:1, and the altered archaeal Family B DNA polymerase is capable of incorporating a modified nucleotide comprising a 3′-OH acetal blocking group or a 3′-OH thiocarbamate blocking group at (i) a lower error rate, (ii) a lower phasing rate, or both (i) and (ii), compared to SEQ ID NO:1.

Aspect 37 is the altered archaeal Family B DNA polymerase of aspect 36, wherein the substitution mutation at the position functionally equivalent to Phe405 comprises a mutation to a non-polar or hydrophobic amino acid.

Aspect 38 is the altered archaeal Family B DNA polymerase of aspect 36 or 37, wherein the substitution mutation at the position functionally equivalent to Phe405 comprises a mutation to Met.

Aspect 39 is the altered archaeal Family B DNA polymerase of any of aspects 36-38, wherein the substitution mutation at the position functionally equivalent to Val485 comprises a mutation to a non-polar or hydrophobic or uncharged amino acid.

Aspect 40 is the altered archaeal Family B DNA polymerase of any of aspects 36-39, wherein the substitution mutation at the position functionally equivalent to Val485 comprises a mutation to Gly.

Aspect 41 is the altered archaeal Family B DNA polymerase of aspect any of aspects 36-40, wherein the substitution mutation at the position functionally equivalent to Phe405 comprises a mutation to Met and the substitution mutation at the position functionally equivalent to Val485 comprises a mutation to Gly.

Aspect 42 is the altered archaeal Family B DNA polymerase of any of aspects 36-41, the polymerase further comprising an amino acid substitution mutation at a position functionally equivalent to Ala408, Ile410, or both Ala408 and Ile410, in the reference archaeal Family B DNA polymerase.

Aspect 43 is the altered archaeal Family B DNA polymerase of any of aspects 36-42, wherein the substitution mutation at the position functionally equivalent to Ala408 comprises a mutation to a polar or uncharged amino acid.

Aspect 44 is the altered archaeal Family B DNA polymerase of any of aspects 36-43, wherein the substitution mutation at the position functionally equivalent to Ala408 comprises a mutation to Ser.

Aspect 45 is the altered archaeal Family B DNA polymerase of any of aspects 36-44, wherein the substitution mutation at the position functionally equivalent to Ile410 comprises a mutation to a non-polar or hydrophobic amino acid.

Aspect 46 is the altered archaeal Family B DNA polymerase of any of aspects 36-45, wherein the substitution mutation at the position functionally equivalent to Ile410 comprises a mutation to Val.

Aspect 47 is the altered archaeal Family B DNA polymerase of any of aspects 36-46, wherein the substitution mutation at the position functionally equivalent to Phe405 comprises a mutation to Met and the substitution mutation at the position functionally equivalent to Val485 comprises a mutation to Gly, further comprising a substitution mutation at a position functionally equivalent to Ala408 that comprises a mutation to Ser and a substitution mutation at a position functionally equivalent to Ile410 that comprises a mutation to Val.

Aspect 48 is the altered archaeal Family B DNA polymerase of any of aspects 36-47, wherein the substitution mutation at the position functionally equivalent to Phe405 comprises a mutation to Met and the substitution mutation at the position functionally equivalent to Val485 comprises a mutation to Gly, further comprising a substitution mutation at a position functionally equivalent to Ala408 that comprises a mutation to Ser.

Aspect 49 is an altered archaeal Family B DNA polymerase comprising an amino acid substitution mutation at a position functionally equivalent to Phe405 and Ile410 in a reference archaeal Family B DNA polymerase of SEQ ID NO:1, and the altered archaeal Family B DNA polymerase is capable of incorporating a modified nucleotide comprising a 3′-OH acetal blocking group or a 3′-OH thiocarbamate blocking group at (i) a lower error rate, (ii) a lower phasing rate, or both (i) and (ii), compared to SEQ ID NO:1.

Aspect 50 is the altered archaeal Family B DNA polymerase of aspect 49, wherein the substitution mutation at the position functionally equivalent to Phe405 comprises a mutation to a non-polar or hydrophobic amino acid.

Aspect 51 is the altered archaeal Family B DNA polymerase of aspect 49 or 50, wherein the substitution mutation at the position functionally equivalent to Phe405 comprises a mutation to Met.

Aspect 52 is the altered archaeal Family B DNA polymerase of any of aspects 49-51, wherein the substitution mutation at the position functionally equivalent to Ile410 comprises a mutation to a non-polar amino acid.

Aspect 53 is the altered archaeal Family B DNA polymerase of any of aspects 49-52, wherein the substitution mutation at the position functionally equivalent to Ile410 comprises a mutation to Val.

Aspect 54 is the altered archaeal Family B DNA polymerase of any of aspects 49-53, wherein the substitution mutation at the position functionally equivalent to Phe405 comprises a mutation to Met and the substitution mutation at the position functionally equivalent to Ile410 comprises a mutation to Val.

Aspect 55 is an altered archaeal Family B DNA polymerase comprising an amino acid substitution mutation at a position functionally equivalent to Phe140 and Ser407 in a reference archaeal Family B DNA polymerase of SEQ ID NO:1, and the altered archaeal Family B DNA polymerase is capable of incorporating a modified nucleotide comprising a 3′-OH acetal blocking group or a 3′-OH thiocarbamate blocking group at (i) a lower error rate, (ii) a lower phasing rate, or both (i) and (ii), compared to SEQ ID NO:1.

Aspect 56 is the altered archaeal Family B DNA polymerase of any of aspects 49-55, wherein the substitution mutation at the position functionally equivalent to Phe140 comprises a mutation to a non-polar or hydrophobic amino acid.

Aspect 57 is the altered archaeal Family B DNA polymerase of any of aspects 49-56, wherein the substitution mutation at the position functionally equivalent to Phe140 comprises a mutation to Leu.

Aspect 58 is the altered archaeal Family B DNA polymerase of any of aspects 49-57, wherein the substitution mutation at the position functionally equivalent to Ser407 comprises a mutation to a non-polar or hydrophobic amino acid.

Aspect 59 is the altered archaeal Family B DNA polymerase of any of aspects 49-58, wherein the substitution mutation at the position functionally equivalent to Ser407 comprises a mutation to Leu.

Aspect 60 is the altered archaeal Family B DNA polymerase of any of aspects 49-59, wherein the substitution mutation at the position functionally equivalent to Phe405 comprises a mutation to Leu and the substitution mutation at the position functionally equivalent to Ser407 comprises a mutation to Leu.

Aspect 61 is an altered archaeal Family B DNA polymerase comprising an amino acid substitution mutation at a position functionally equivalent to Leu403, Ala408, Ile410, and Gly497 in a reference archaeal Family B DNA polymerase of SEQ ID NO: 1, and the altered archaeal Family B DNA polymerase is capable of incorporating a modified nucleotide comprising a 3′-OH acetal blocking group or a 3′-OH thiocarbamate blocking group at (i) a lower error rate, (ii) a lower phasing rate, or both (i) and (ii), compared to SEQ ID NO:1.

Aspect 62 is the altered archaeal Family B DNA polymerase of aspect 61, wherein the substitution mutation at the position functionally equivalent to Leu403 comprises a mutation to a non-polar or hydrophobic amino acid.

Aspect 63 is the altered archaeal Family B DNA polymerase of aspect 61 or 62, wherein the substitution mutation at the position functionally equivalent to Leu403 comprises a mutation to Met.

Aspect 64 is the altered archaeal Family B DNA polymerase of any of aspects 61-63, wherein the substitution mutation at the position functionally equivalent to Ala408 comprises a mutation to a polar or uncharged amino acid.

Aspect 65 is the altered archaeal Family B DNA polymerase of any of aspects 61-64, wherein the substitution mutation at the position functionally equivalent to Ala408 comprises a mutation to Ser.

Aspect 66 is the altered archaeal Family B DNA polymerase of any of aspects 61-65, wherein the substitution mutation at the position functionally equivalent to Ile410 comprises a mutation to a non-polar or hydrophobic amino acid.

Aspect 67 is the altered archaeal Family B DNA polymerase of any of aspects 61-66, wherein the substitution mutation at the position functionally equivalent to Ile410 comprises a mutation to Val.

Aspect 68 is the altered archaeal Family B DNA polymerase of any of aspects 61-67, wherein the substitution mutation at the position functionally equivalent to Gly497 comprises a mutation to a non-polar or hydrophobic amino acid.

Aspect 69 is the altered archaeal Family B DNA polymerase of any of aspects 61-68, wherein the substitution mutation at the position functionally equivalent to Gly497 comprises a mutation to Met.

Aspect 70 is the altered archaeal Family B DNA polymerase of any of aspects 61-69, wherein the substitution mutation at the position functionally equivalent to Leu403 comprises a mutation to Met, the substitution mutation at the position functionally equivalent to Ala408 comprises a mutation to Ser, the substitution mutation at the position functionally equivalent to Ile410 comprises a mutation to Val, and the substitution mutation at the position functionally equivalent to Gly497 comprises a mutation to Met.

Aspect 71 is an altered archaeal Family B DNA polymerase comprising an amino acid substitution mutation at a position functionally equivalent to Phe405, Ile410, Ile412, Thr514, and Ile521 in a reference archaeal Family B DNA polymerase of SEQ ID NO:1, and the altered archaeal Family B DNA polymerase is capable of incorporating a modified nucleotide comprising a 3′-OH acetal blocking group or a 3′-OH thiocarbamate blocking group at (i) a lower error rate, (ii) a lower phasing rate, or both (i) and (ii), compared to SEQ ID NO:1.

Aspect 72 is the altered archaeal Family B DNA polymerase of aspect 71, wherein the substitution mutation at the position functionally equivalent to Phe405 comprises a mutation to a non-polar or hydrophobic amino acid.

Aspect 73 is the altered archaeal Family B DNA polymerase of aspect 71 or 72, wherein the substitution mutation at the position functionally equivalent to Phe405 comprises a mutation to Ile.

Aspect 74 is the altered archaeal Family B DNA polymerase of any of aspects 71-73, wherein the substitution mutation at the position functionally equivalent to Ile410 comprises a mutation to a non-polar or hydrophobic amino acid.

Aspect 75 is the altered archaeal Family B DNA polymerase of any of aspects 71-74, wherein the substitution mutation at the position functionally equivalent to Ile410 comprises a mutation to Pro.

Aspect 76 is the altered archaeal Family B DNA polymerase of any of aspects 71-75, wherein the substitution mutation at the position functionally equivalent to Ile412 comprises a mutation to a non-polar or hydrophobic amino acid.

Aspect 77 is the altered archaeal Family B DNA polymerase of any of aspects 71-76, wherein the substitution mutation at the position functionally equivalent to Ile412 comprises a mutation to Met.

Aspect 78 is the altered archaeal Family B DNA polymerase of any of aspects 71-77, wherein the substitution mutation at the position functionally equivalent to Thr514 comprises a mutation to a non-polar or hydrophobic amino acid.

Aspect 79 is the altered archaeal Family B DNA polymerase of any of aspects 71-78, wherein the substitution mutation at the position functionally equivalent to Thr514 comprises a mutation to Ala.

Aspect 80 is the altered archaeal Family B DNA polymerase of any of aspects 71-79, wherein the substitution mutation at the position functionally equivalent to Ile521 comprises a mutation to a non-polar or hydrophobic amino acid.

Aspect 81 is the altered archaeal Family B DNA polymerase of any of aspects 71-80, wherein the substitution mutation at the position functionally equivalent to Ile521 comprises a mutation to Ala.

Aspect 82 is the altered archaeal Family B DNA polymerase of any of aspects 71-81, wherein the substitution mutation at the position functionally equivalent to Phe405 comprises a mutation to Ile, the substitution mutation at the position functionally equivalent to Ile410 comprises a mutation to Pro, the substitution mutation at the position functionally equivalent to Ile412 comprises a mutation to Met, the substitution mutation at the position functionally equivalent to Thr514 comprises a mutation to Ala, and the substitution mutation at the position functionally equivalent to Ile521 comprises a mutation to Ala.

Aspect 83 is an altered archaeal Family B DNA polymerase comprising an amino acid substitution mutation at a position functionally equivalent to Phe405, Ala408, Ile410, Ile412, Thr514, Ile521 in a reference archaeal Family B DNA polymerase of SEQ ID NO:1, and the altered archaeal Family B DNA polymerase is capable of incorporating a modified nucleotide comprising a 3′-OH acetal blocking group or a 3′-OH thiocarbamate blocking group at (i) a lower error rate, (ii) a lower phasing rate, or both (i) and (ii), compared to SEQ ID NO:1.

Aspect 84 is the altered archaeal Family B DNA polymerase of aspect 83, wherein the substitution mutation at the position functionally equivalent to Phe405 comprises a mutation to a non-polar or hydrophobic amino acid.

Aspect 85 is the altered archaeal Family B DNA polymerase of aspect 83 or 84, wherein the substitution mutation at the position functionally equivalent to Phe405 comprises a mutation to Leu.

Aspect 86 is the altered archaeal Family B DNA polymerase of any of aspects 83-85, wherein the substitution mutation at the position functionally equivalent to Ala408 comprises a mutation to a polar or uncharged amino acid.

Aspect 87 is the altered archaeal Family B DNA polymerase of any of aspects 83-86, wherein the substitution mutation at the position functionally equivalent to Ala408 comprises a mutation to Ser.

Aspect 88 is the altered archaeal Family B DNA polymerase of any of aspects 83-87, wherein the substitution mutation at the position functionally equivalent to Ile410 comprises a mutation to a non-polar or hydrophobic amino acid.

Aspect 89 is the altered archaeal Family B DNA polymerase of any of aspects 83-88, wherein the substitution mutation at the position functionally equivalent to Ile410 comprises a mutation to Val.

Aspect 90 is the altered archaeal Family B DNA polymerase of any of aspects 83-89, wherein the substitution mutation at the position functionally equivalent to Ile412 comprises a mutation to a polar or uncharged amino acid.

Aspect 91 is the altered archaeal Family B DNA polymerase of any of aspects 83-90, wherein the substitution mutation at the position functionally equivalent to Ile412 comprises a mutation to Thr.

Aspect 92 is the altered archaeal Family B DNA polymerase of any of aspects 83-91, wherein the substitution mutation at the position functionally equivalent to Thr514 comprises a mutation to a non-polar or hydrophobic amino acid.

Aspect 93 is the altered archaeal Family B DNA polymerase of any of aspects 83-92, wherein the substitution mutation at the position functionally equivalent to Thr514 comprises a mutation to Ala.

Aspect 94 is the altered archaeal Family B DNA polymerase of any of aspects 83-93, wherein the substitution mutation at the position functionally equivalent to Ile521 comprises a mutation to a non-polar or hydrophobic amino acid.

Aspect 95 is the altered archaeal Family B DNA polymerase of any of aspects 83-94, wherein the substitution mutation at the position functionally equivalent to Ile521 comprises a mutation to Ala.

Aspect 96 is the altered archaeal Family B DNA polymerase of any of aspects 83-95, wherein the substitution mutation at the position functionally equivalent to Phe405 comprises a mutation to Leu, the substitution mutation at the position functionally equivalent to Ala408 comprises a mutation to Ser, the substitution mutation at the position functionally equivalent to Ile410 comprises a mutation to Val, the substitution mutation at the position functionally equivalent to Ile412 comprises a mutation to Thr, the substitution mutation at the position functionally equivalent to Thr514 comprises a mutation to Ala, and the substitution mutation at the position functionally equivalent to Ile521 comprises a mutation to Ala.

Aspect 97 is an altered archaeal Family B DNA polymerase comprising the amino acid sequence of any of SEQ ID NOs:2-8 or any of SEQ ID NOs: 16-32.

Aspect 98 is the altered archaeal Family B DNA polymerase of any of aspects 1-97, wherein the polymerase comprises reduced exonuclease activity as compared to SEQ ID NO:9.

Aspect 99 is a nucleic acid molecule encoding a DNA polymerase as defined in any of aspects 1-98.

Aspect 100 is an expression vector comprising the nucleic acid molecule of aspect 99.

Aspect 101 is a host cell comprising the vector of aspect 100.

Aspect 102 is a method for incorporating modified nucleotides into a polynucleotide complementary to a target nucleic acid comprising allowing the following components to interact: (i) the altered archaeal Family B DNA polymerase of one of aspects 1-97, (ii) a DNA template; and (iii) a nucleotide solution.

Aspect 103 is the method of aspect 102, wherein the DNA template comprises a clustered array.

Aspect 104 is a kit for performing a nucleotide incorporation reaction comprising: the altered archaeal Family B DNA polymerase of any of aspects 1-97, and a solution comprising second generation fully functional nucleotides.

Aspect 105 is the kit of aspect 104, wherein the second generation fully functional nucleotides comprise a detectable label.

Aspect 106 is the kit of aspect 104 or 105, wherein the second generation fully functional nucleotides have been modified at the 3′ sugar hydroxyl such that the substituent is larger in size than the naturally occurring 3′ hydroxyl group.

Aspect 107 is the kit of any of aspects 104-106, wherein the second generation fully functional nucleotides comprise a modified nucleotide molecule comprising a purine or pyrimidine base and a deoxyribose sugar moiety comprising a removable 3′-OH acetal blocking group or a 3′-OH thiocarbamate blocking group attached to the 3′ carbon of the deoxyribose sugar moiety.

Aspect 108 is the kit of any of aspects 104-107, wherein the 3′-OH acetal blocking group has the structure

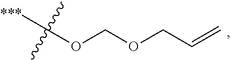

wherein the *** indicates the attachment point of the 3′-OH acetal blocking group to the 3′ carbon of the modified nucleotide sugar.

Aspect 109 is the kit of any of aspects 104-108, wherein the second generation fully functional nucleotides comprise a nucleotide molecule comprising a purine or pyrimidine base attached to a detectable label via a cleavable linker.

Aspect 110 is the kit of any of aspects 104-109, wherein the detectable label comprises a fluorescent label.

Aspect 111 is the kit of any of aspects 104-110, wherein the cleavable linker is selected from the group consisting of

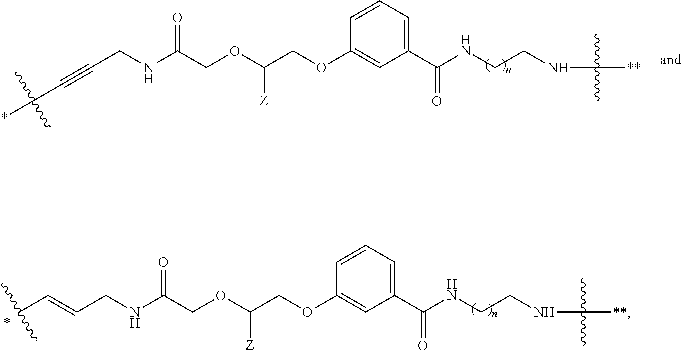

wherein Z is —O—CH₂-CH-CH2; n is an integer of 1, 2, 3, 4 or 5; * indicates the attachment point of the cleavable linker to the purine or pyrimidine base; and ** indicates the attachment point of the cleavable linker to the detectable label.

Aspect 112 is the kit of any of aspects 104-111, further comprising one or more DNA template molecules and/or primers.

### EXAMPLES

The present disclosure is illustrated by the following examples. It is to be understood that the particular examples, materials, amounts, and procedures are to be interpreted broadly in accordance with the scope and spirit of the disclosure as set forth herein.

Sequencing experiments were used to compare error rates, phasing values. Unless indicated otherwise, the experiments were carried out on a MiniSeq™ system (Illumina, Inc., San Diego, Calif.), For example, for each polymerase, a separate incorporation mix (IMX) was prepared and used in a short reads at either 30 second or 20 second allowed reaction times. In case of the short reads, the use of a modified kit allowed for 6 to 9 distinct incorporation mixes. Modifications to the kit included replacing the deblocking reagent and deblock scavenger wash buffer. In these experiments, first and last reads contained the internal controls for analysis of phasing values and error rates to assess polymerase performance. The first read and second read was used to establish baseline performance of the control polymerase and custom IMX formulation. The last read was used to assess potential quality degradation throughout the duration of the run. Controls used were SEQ ID NO: 1 when polymerases with a single substitution mutation were compared in a sequencing run, and SEQ ID NO:2 when polymerases with two or more substitution mutations were compared in a sequencing run.

### Primary Screening

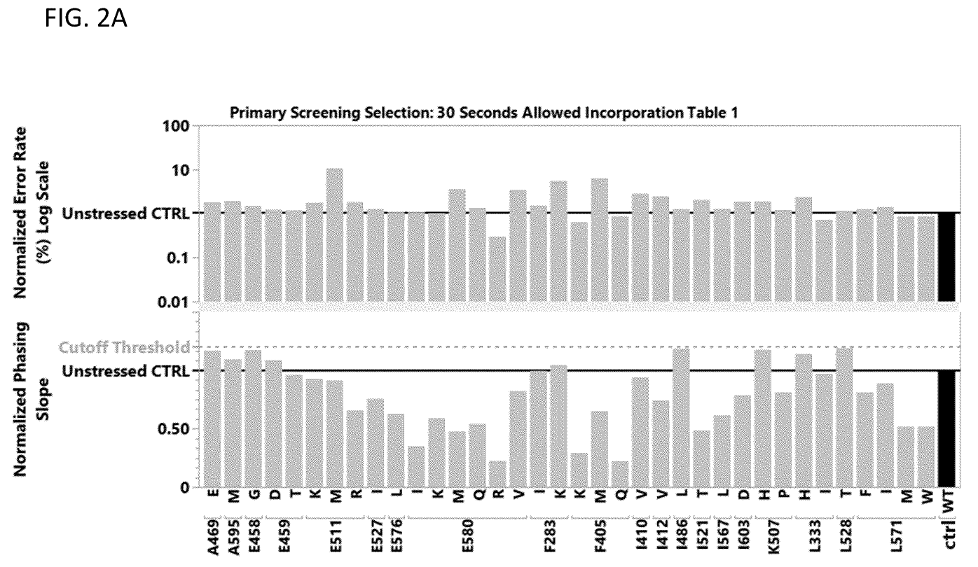

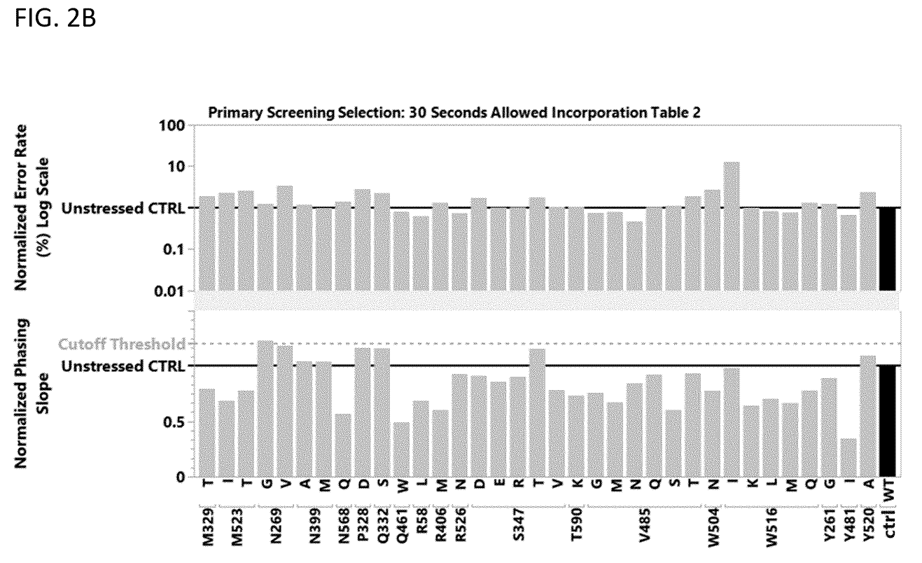

Sequencing experiments were used to compare error rates, phasing values. Unless indicated otherwise, the experiments were carried out on a MiniSeq™ system (Illumina, Inc., San Diego, Calif.), For example, for each polymerase, a separate incorporation mix (IMX) was prepared and used in a short reads at 30 second allowed reaction times. Modified MiniSeq Mid Output Reagent Cartridge formulations were used, with the standard polymerase substituted with the polymerase being tested. The use of the modified kit allowed for 6 to 9 distinct incorporation mixes. In these experiments, first and last reads contained the internal controls for analysis of phasing values and error rates to assess polymerase performance. The first read was used to establish baseline performance of the control polymerase and custom IMX formulation. The last read was used to assess potential quality degradation throughout the duration of the run. Observed phasing values and error rates were reported for each 36 cycle read containing distinct polymerases. Phasing and error rate values for mutants were normalized to the Read 1 control by dividing observed phasing and error rates observed by the phasing and error rates of the Read 1 control. Mutants with normalized phasing values equal to or less than 1.2 were selected for secondary screening (FIG. 2).

### Secondary Screening

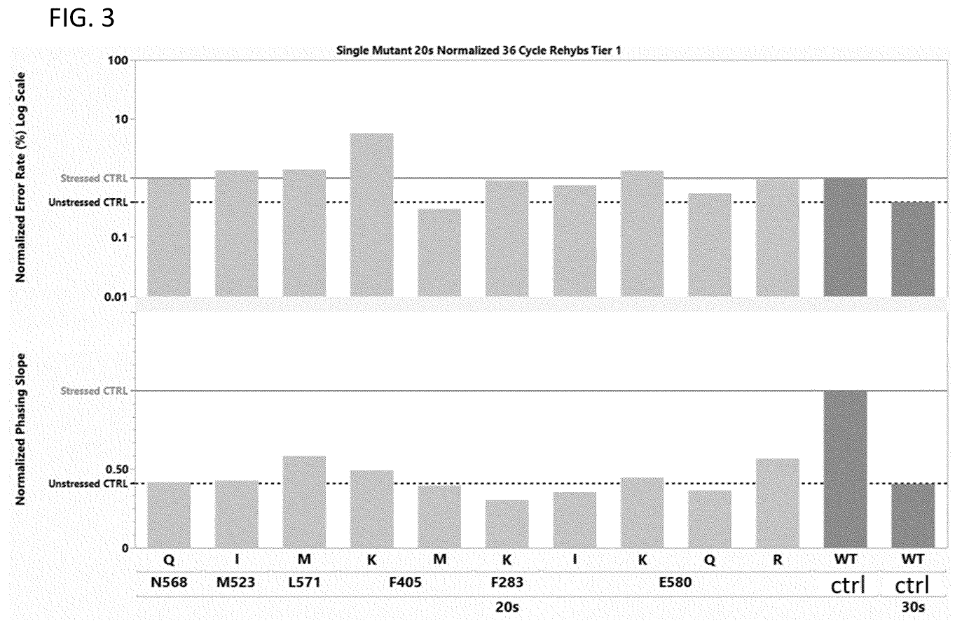

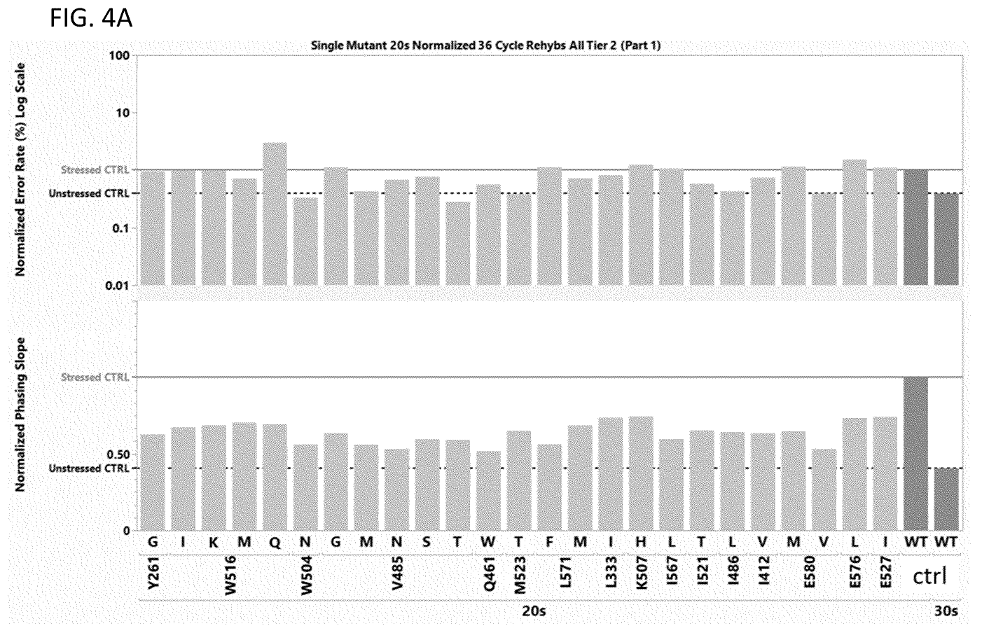

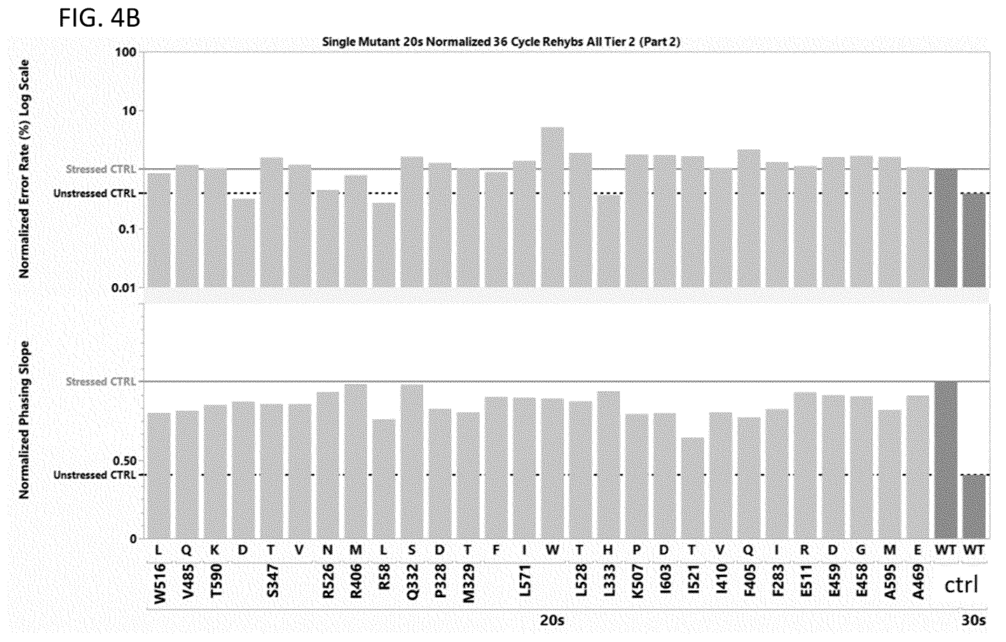

Sequencing experiments were used to compare error rates and phasing values. Unless indicated otherwise, the experiments were carried out on a MiniSeq™ system (Illumina, Inc., San Diego, Calif.), For example, for each polymerase, a separate incorporation mix (IMX) was prepared and used in a short reads at 30 second allowed reaction times. Modified MiniSeq Mid Output Reagent Cartridge formulations were used, with the standard polymerase substituted with the polymerase being tested. The use of the modified kit allowed for 6 to 9 distinct incorporation mixes. In these experiments, first, second and last reads contained the internal controls for analysis of phasing values and error rates to assess polymerase performance. The first read was used to establish baseline performance of the control polymerase and custom IMX formulation under permissive conditions, with a 30 second allowed reaction time. The Second read was used to establish performance of the control polymerase and custom IMX formulation under stressed conditions, with a 20 second allowed reaction time. The last read was used to assess potential quality degradation throughout the duration of the run, also under stressed conditions, with a 20 second allowed reaction time. Observed phasing values and error rates were reported for each 36 cycle read containing distinct polymerases. Phasing and error rate values for mutants were normalized to the Read 2 control. Normalized values were obtained by dividing observed phasing and error rates observed by the phasing and error rates of the Read 2 control. Mutants with an average normalized value less than or equal to 0.5 were classed as Tier 1 (FIG. 3). Mutants with an average normalized value greater than 0.5 and less than or equal to 1.0 were classed as Tier 2 (FIG. 4). Mutants with an average normalized phasing values greater than 1.0 were not classed.

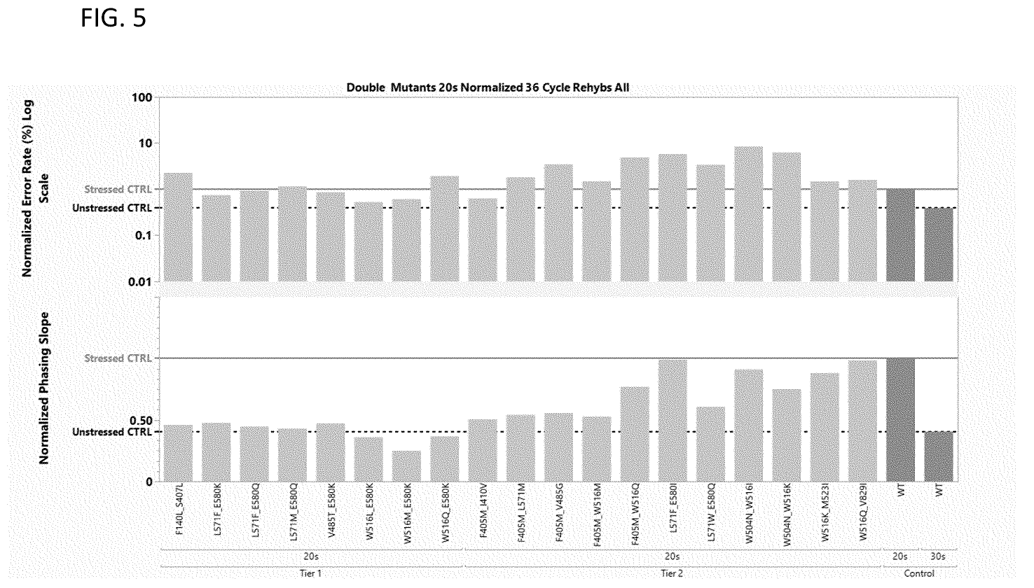

The Tier 1 and Tier 2 mutations were recombined to further identify any additional improvements to sequencing performance—some exemplary double mutants are shown in FIG. 5.

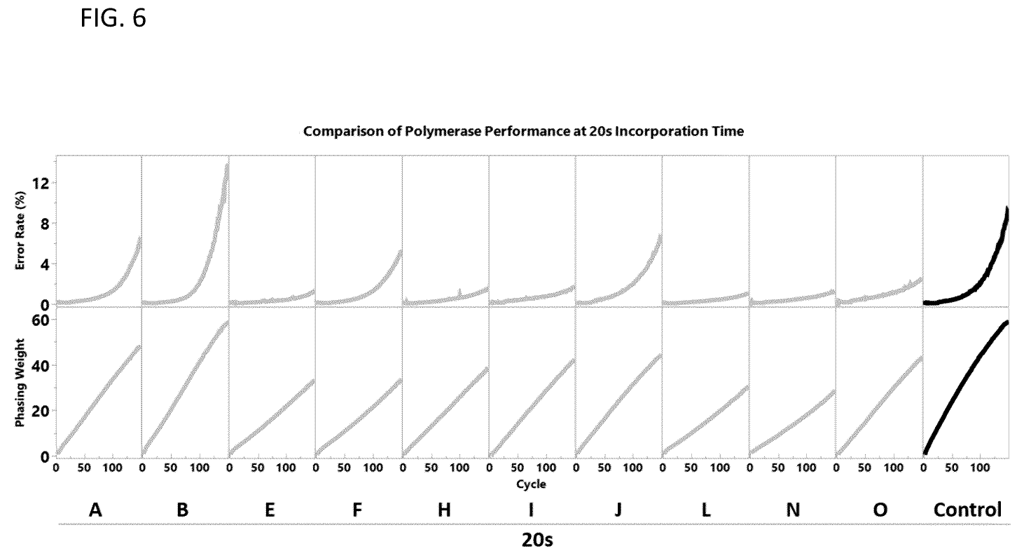

Last, in some instances these double mutants or similar recombinations may have been used as a new backbone to which further mutations were added prior to validation in 150 cycle runs (FIG. 6) examples of 151 cycle validation runs.

Validation runs (150 cycle)

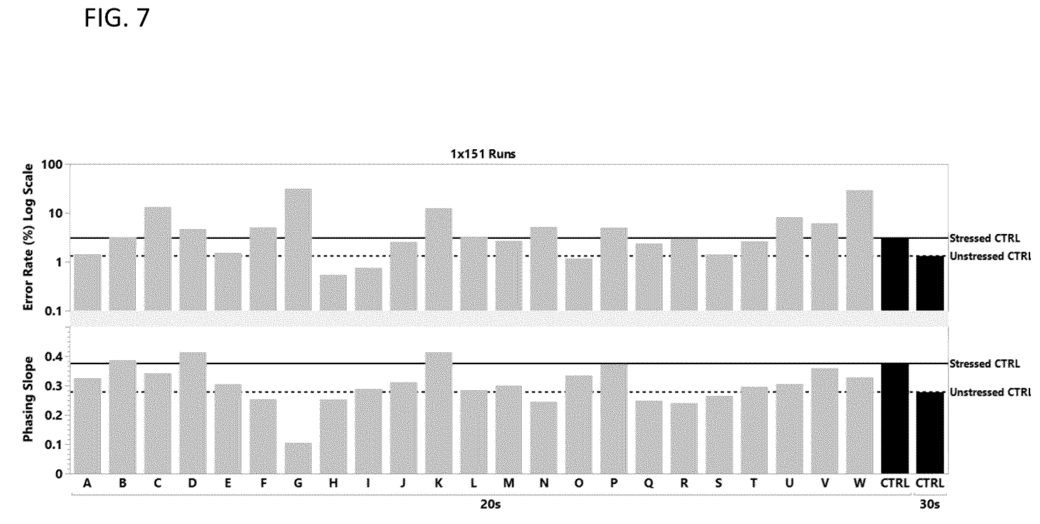

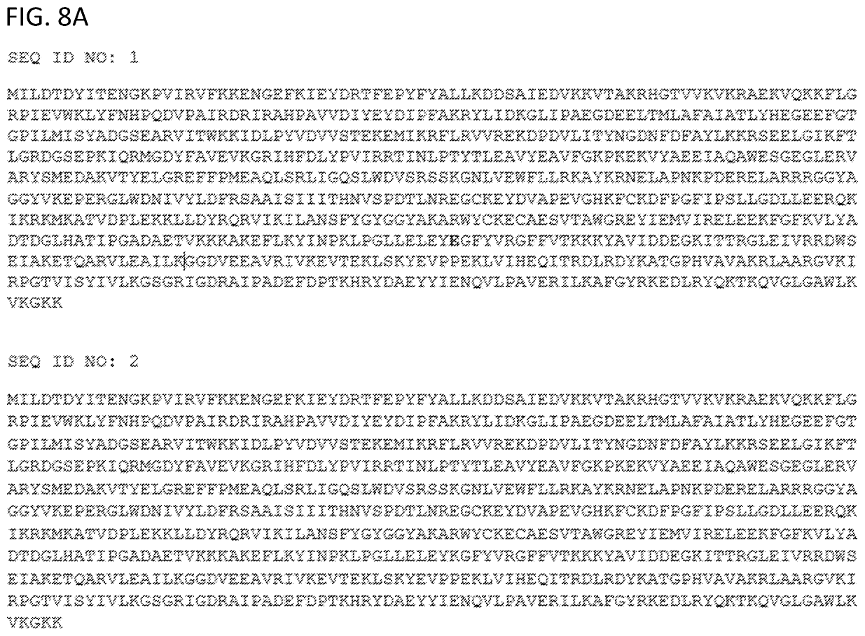

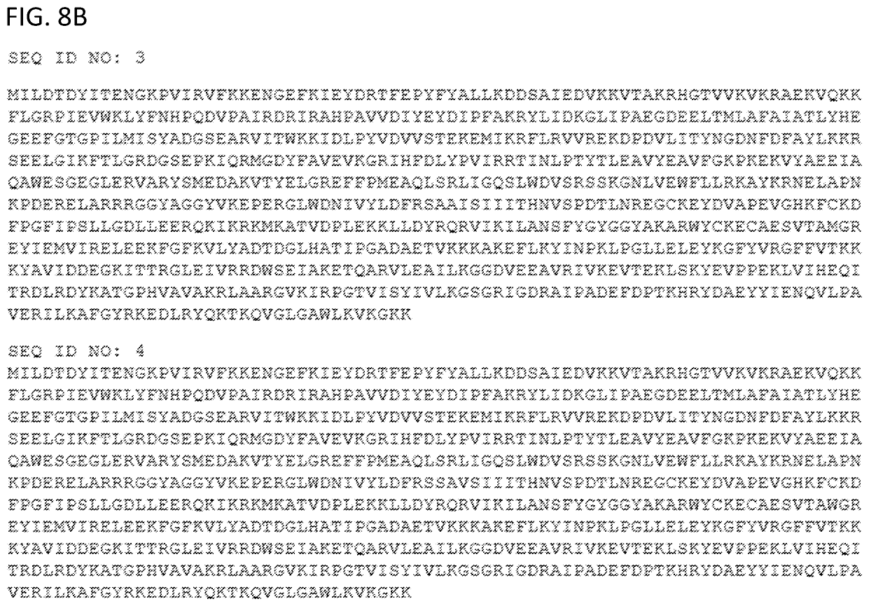

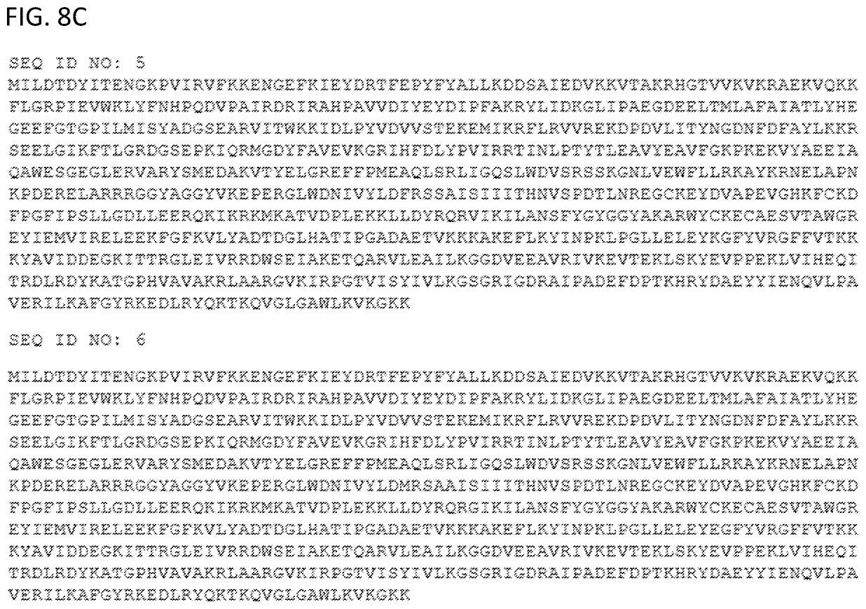

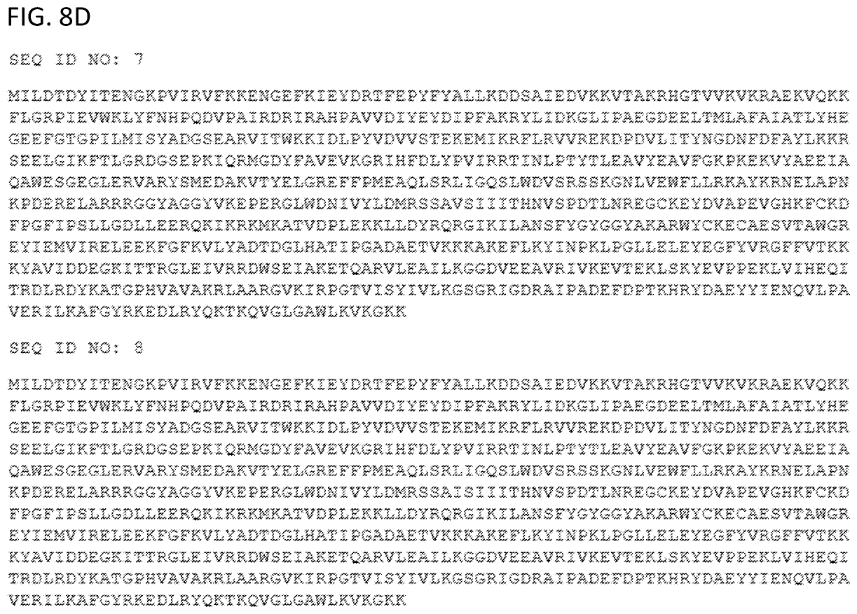

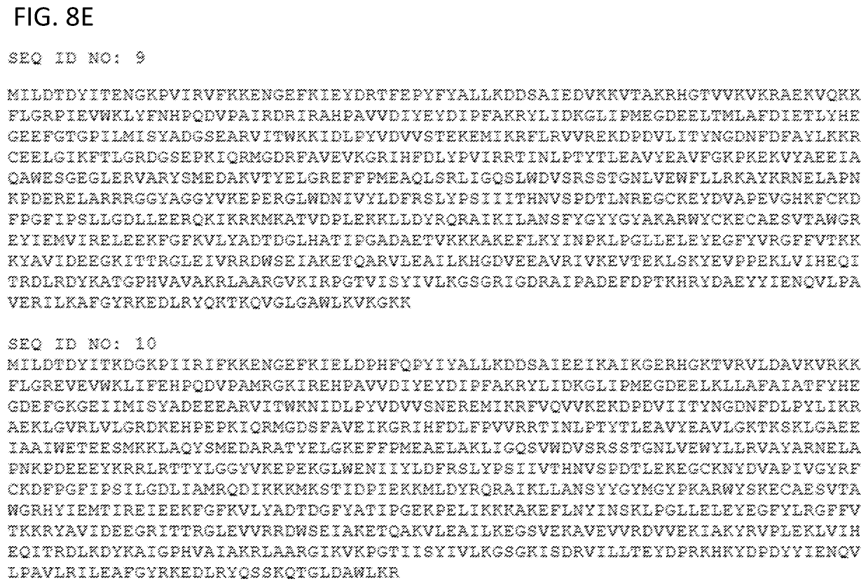

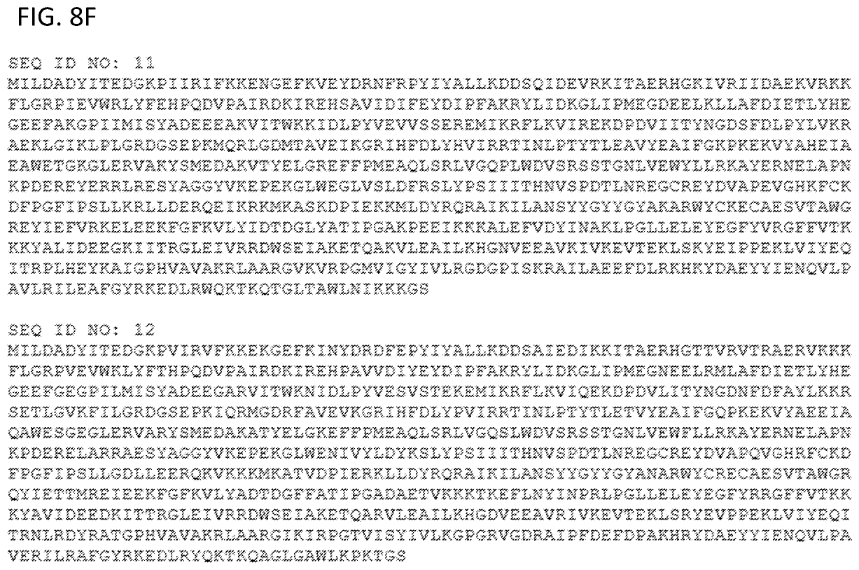

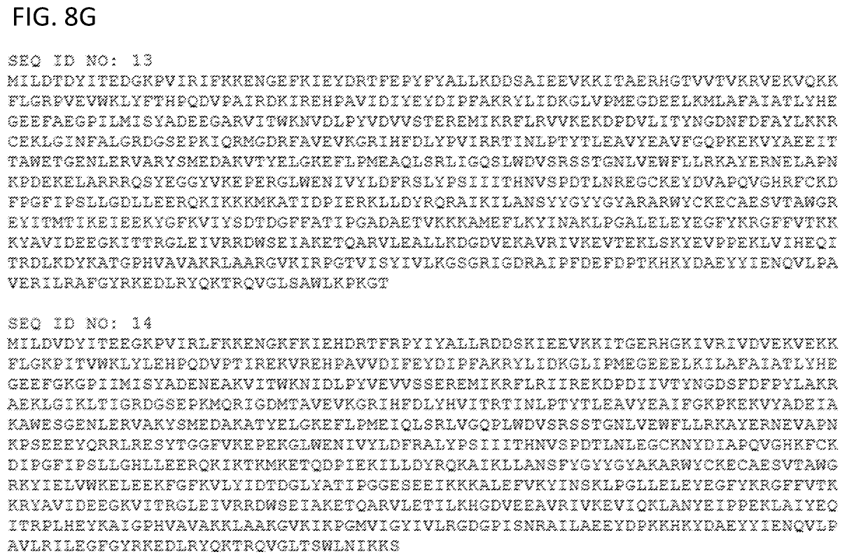

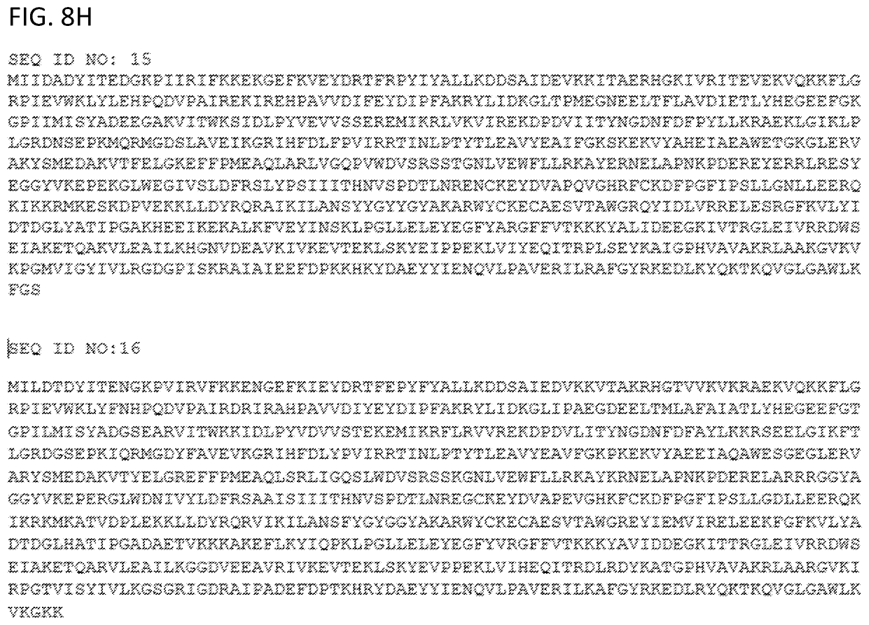

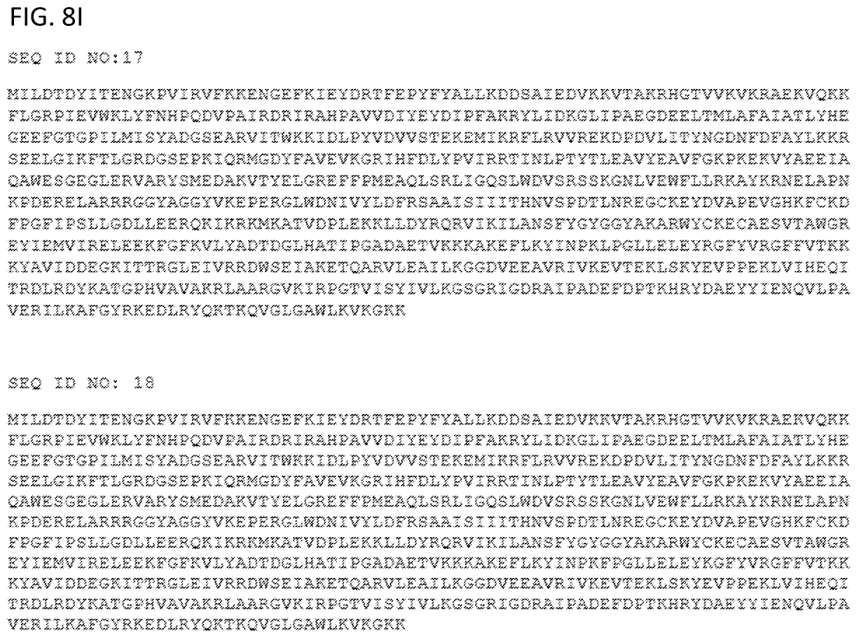

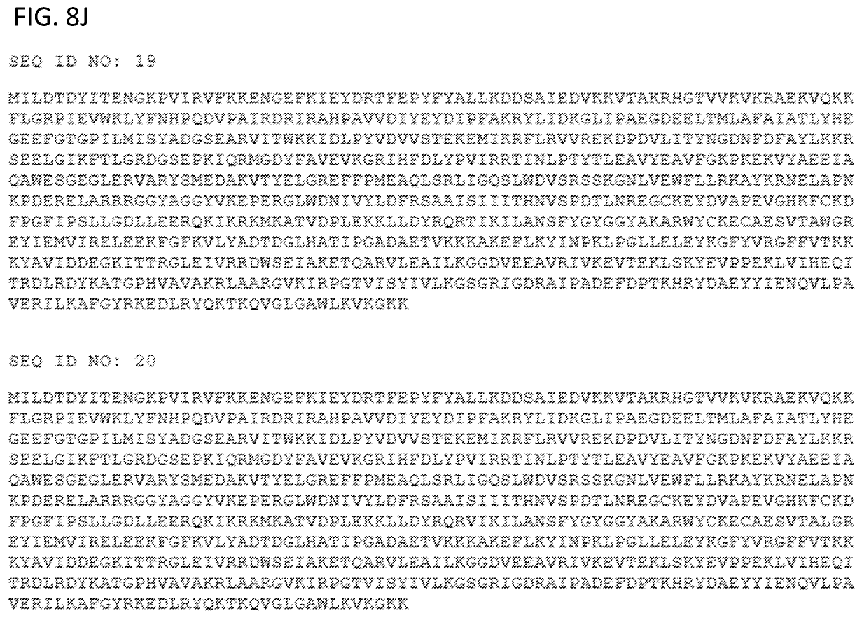

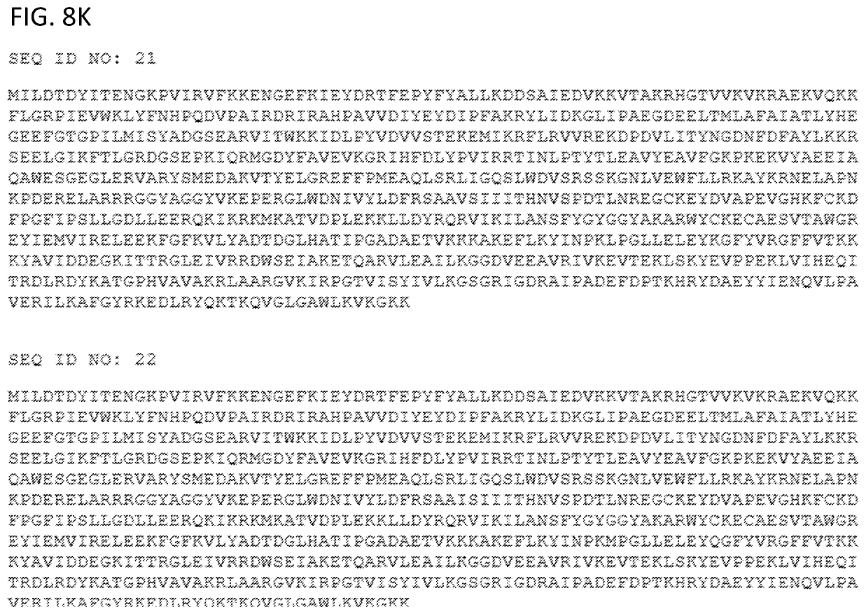

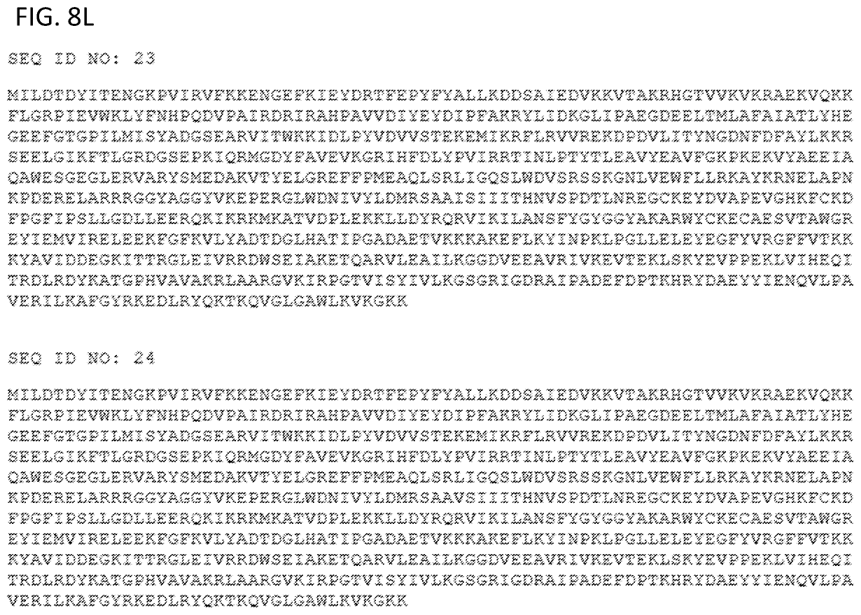

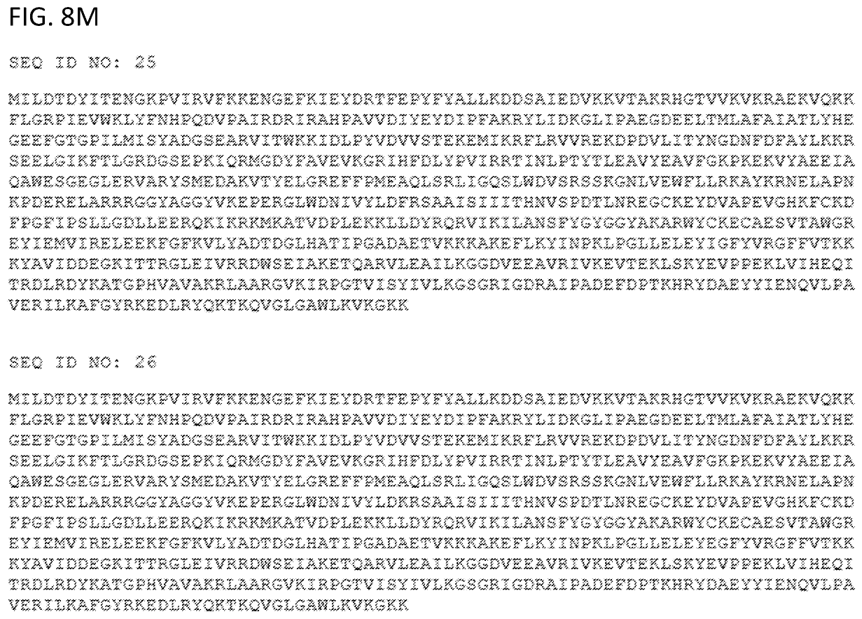

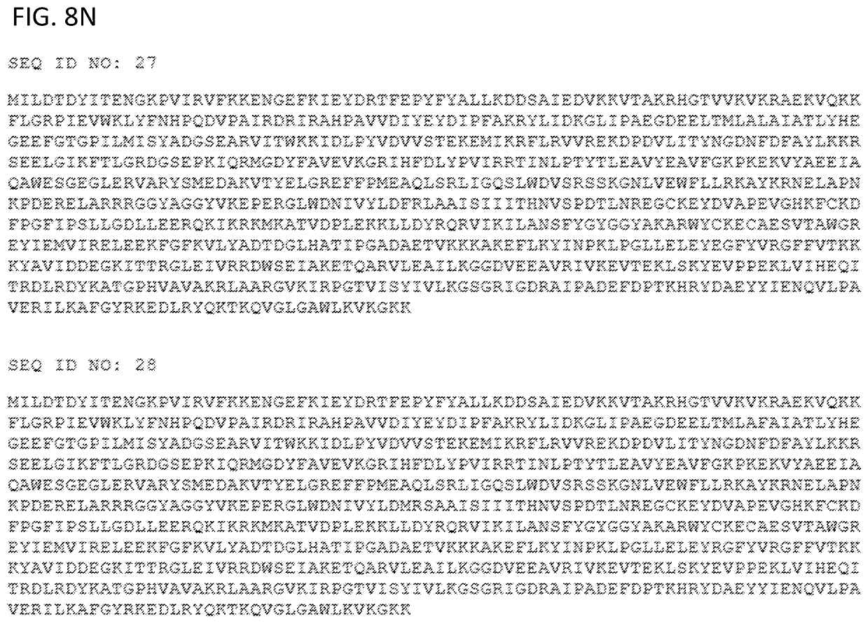

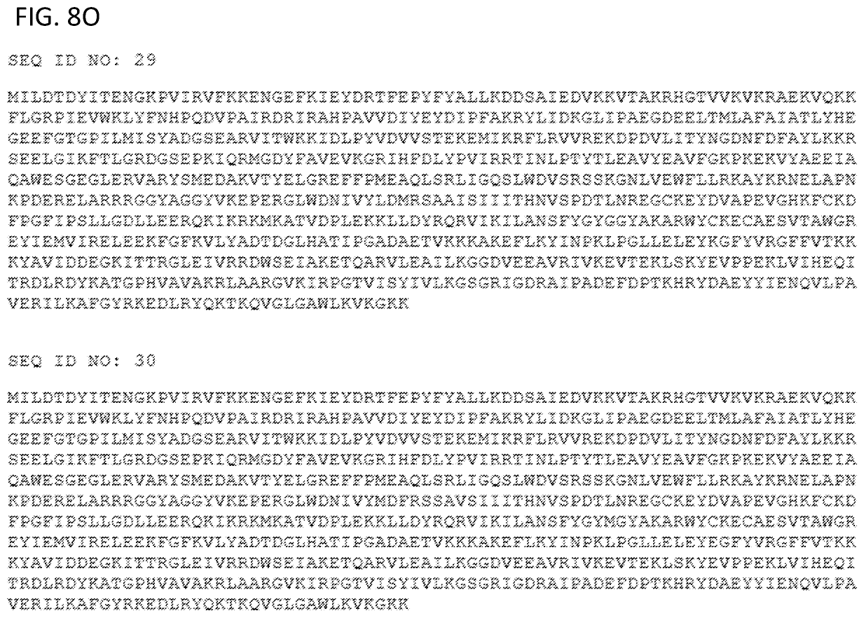

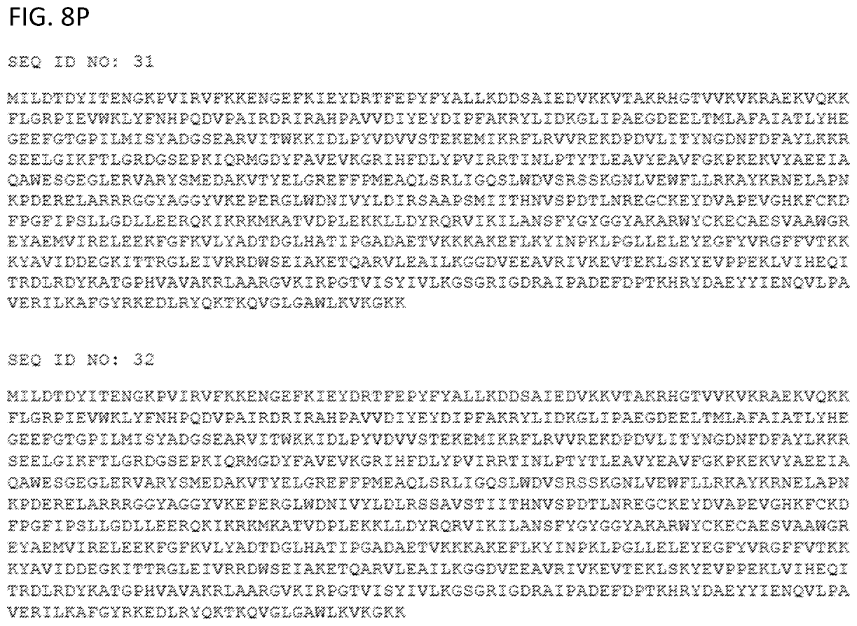

In some instances, polymerases were prepared for long runs (FIG. 7). Modified MiniSeq Mid Output Reagent Cartridge formulations were used, with the standard polymerase substituted with the polymerase being tested. The time for incubation of IMX on the flowcell varied as noted in the Examples herein. Phasing values and error rates were reported and compared to the values observed for the control under the same conditions as noted.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference in their entirety. Supplementary materials referenced in publications (such as supplementary tables, supplementary figures, supplementary materials and methods, and/or supplementary experimental data) are likewise incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The disclosure is not limited to the exact details shown and described, for variations obvious to one skilled in the art will be included within the disclosure defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present disclosure. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the disclosure are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

1. An altered archaeal Family B DNA polymerase, wherein the altered archaeal Family B DNA polymerase comprises at least one an amino acid substitution mutation at a position functionally equivalent to an amino acid in a reference archaeal Family B DNA polymerase of SEQ ID NO:1, and the altered archaeal Family B DNA polymerase is capable of incorporating a modified nucleotide comprising a 3′-OH acetal blocking group or a 3′-OH thiocarbamate blocking group at (i) a lower error rate, (ii) a lower phasing rate, or both (i) and (ii), compared to SEQ ID NO:1;
wherein the amino acid is at position Phe405
wherein the amino acid is at position Glu580, and a second amino acid is at position Phe405;
wherein the amino acid is at position Glu580, and a second amino acid is at position Val485;
wherein the amino acid is at position Glu580, and a second amino acid is at position Trp516;
wherein the amino acid is at position Glu580, and a second amino acid is at position Leu571;
wherein the amino acid is at position Glu580, and a second amino acid is at position Leu571;
wherein the amino acid is at position Glu580, a second amino acid is at position Ala408, and a third mutation is at position Ile410;
wherein the amino acid is at position Glu580, and a second amino acid is at position Ala408;
wherein the amino acid is at position Glu580, and a second amino acid is at position Phe405;
wherein the amino acid is at position Glu580, and a second amino acid is at position Ile410;
wherein the amino acid is at position Phe405, and a second amino acid is at position Val485;
wherein the amino acid is at position Phe405, a second amino acid is at position Val485, and a third mutation is at position Ala408;
wherein the amino acid is at position Phe405, and a second amino acid is at position Ile410;
wherein the amino acid is at position Phe140, and a second amino acid is at position Ser407;
wherein the amino acid is at position Leu403, a second amino acid is at position Ala408, a third amino acid is at position Ile410, and a fourth amino acid is at position Gly497;
wherein the amino acid is at position Phe405, a second amino acid is at position Ile410, a third amino acid is at position Ile412, a fourth amino acid is at position Thr514, and a fifth amino acid is at position Ile521; or
wherein the amino acid is at position Phe405, a second amino acid is at position Ala408, a third amino acid is at position Ile410, a fourth amino acid is at position Ile412, a fifth amino acid is at position Thr514, and a sixth amino acid is at position Ile521.

2-96. (canceled)

97. An altered archaeal Family B DNA polymerase comprising the amino acid sequence of any one of SEQ ID NOs:2-8 or any one of SEQ ID NOs: 16-32.

98. The altered archaeal Family B DNA polymerase of claim 1, wherein the polymerase comprises reduced exonuclease activity as compared to SEQ ID NO:9.

99. A nucleic acid molecule encoding a DNA polymerase as defined in claim 1.

100. An expression vector comprising the nucleic acid molecule of claim 99.

101. A host cell comprising the vector of claim 100.

102. A method for incorporating modified nucleotides into a polynucleotide complementary to a target nucleic acid comprising allowing the following components to interact: (i) the altered archaeal Family B DNA polymerase of claim 1, (ii) a DNA template; and (iii) a nucleotide solution.

103. The method of claim 102, wherein the DNA template comprises a clustered array.

104. A kit for performing a nucleotide incorporation reaction comprising: the altered archaeal Family B DNA polymerase of claim 1, and a solution comprising second generation fully functional nucleotides.

105. The kit of claim 104, wherein the second generation fully functional nucleotides comprise a detectable label.

106. The kit of claim 104, wherein the second generation fully functional nucleotides have been modified at the 3′ sugar hydroxyl such that the substituent is larger in size than the naturally occurring 3′ hydroxyl group.

107. The kit of claim 106, wherein the second generation fully functional nucleotides comprise a modified nucleotide molecule comprising a purine or pyrimidine base and a deoxyribose sugar moiety comprising a removable 3′-OH acetal blocking group or a 3′-OH thiocarbamate blocking group attached to the 3′ carbon of the deoxyribose sugar moiety.

108. The kit of claim 107, wherein the 3′-OH acetal blocking group has the structure 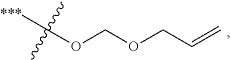
wherein the *** indicates the attachment point of the 3′-OH acetal blocking group to the 3′ carbon of the modified nucleotide sugar.

109. The kit of claim 104, wherein the second generation fully functional nucleotides comprise a nucleotide molecule comprising a purine or pyrimidine base attached to a detectable label via a cleavable linker.

110. The kit of claim 109, wherein the detectable label comprises a fluorescent label.

111. The kit of claim 109, wherein the cleavable linker is selected from the group consisting of 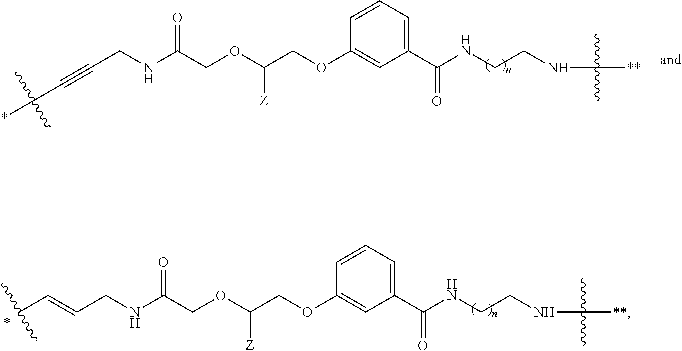
wherein Z is —O—CH₂—CH═CH₂; n is an integer of 1, 2, 3, 4 or 5; * indicates the attachment point of the cleavable linker to the purine or pyrimidine base; and ** indicates the attachment point of the cleavable linker to the detectable label.

112. The kit of claim 104, further comprising one or more DNA template molecules and/or primers.


SEQUENCE LISTING (32 sequences)

SEQ ID NO: 1 - protein, 775 aa, synthetic construct
MILDTDYITENGKPVIRVFKKENGEFKIEYDRTFEPYFYALLKDDSAIEDVKKVTAKRHGTVVKVKRAEKVQKKFLGRPIEVWKLYFNHPQDVPAIRDRIRAHPAVVDIYEYDIPFAKRYLIDKGLIPAEGDEELTMLAFAIATLYHEGEEFGTGPILMISYADGSEARVITWKKIDLPYVDVVSTEKEMIKRFLRVVREKDPDVLITYNGDNFDFAYLKKRSEELGIKFTLGRDGSEPKIQRMGDYFAVEVKGRIHFDLYPVIRRTINLPTYTLEAVYEAVFGKPKEKVYAEEIAQAWESGEGLERVARYSMEDAKVTYELGREFFPMEAQLSRLIGQSLWDVSRSSKGNLVEWFLLRKAYKRNELAPNKPDERELARRRGGYAGGYVKEPERGLWDNIVYLDFRSAAISIIITHNVSPDTLNREGCKEYDVAPEVGHKFCKDFPGFIPSLLGDLLEERQKIKRKMKATVDPLEKKLLDYRQRVIKILANSFYGYGGYAKARWYCKECAESVTAWGREYIEMVIRELEEKFGFKVLYADTDGLHATIPGADAETVKKKAKEFLKYINPKLPGLLELEYEGFYVRGFFVTKKKYAVIDDEGKITTRGLEIVRRDWSEIAKETQARVLEAILKGGDVEEAVRIVKEVTEKLSKYEVPPEKLVIHEQITRDLRDYKATGPHVAVAKRLAARGVKIRPGTVISYIVLKGSGRIGDRAIPADEFDPTKHRYDAEYYIENQVLPAVERILKAFGYRKEDLRYQKTKQVGLGAWLKVKGKK

SEQ ID NO: 2 - protein, 775 aa, synthetic construct
MILDTDYITENGKPVIRVFKKENGEFKIEYDRTFEPYFYALLKDDSAIEDVKKVTAKRHGTVVKVKRAEKVQKKFLGRPIEVWKLYFNHPQDVPAIRDRIRAHPAVVDIYEYDIPFAKRYLIDKGLIPAEGDEELTMLAFAIATLYHEGEEFGTGPILMISYADGSEARVITWKKIDLPYVDVVSTEKEMIKRFLRVVREKDPDVLITYNGDNFDFAYLKKRSEELGIKFTLGRDGSEPKIQRMGDYFAVEVKGRIHFDLYPVIRRTINLPTYTLEAVYEAVFGKPKEKVYAEEIAQAWESGEGLERVARYSMEDAKVTYELGREFFPMEAQLSRLIGQSLWDVSRSSKGNLVEWFLLRKAYKRNELAPNKPDERELARRRGGYAGGYVKEPERGLWDNIVYLDFRSAAISIIITHNVSPDTLNREGCKEYDVAPEVGHKFCKDFPGFIPSLLGDLLEERQKIKRKMKATVDPLEKKLLDYRQRVIKILANSFYGYGGYAKARWYCKECAESVTAWGREYIEMVIRELEEKFGFKVLYADTDGLHATIPGADAETVKKKAKEFLKYINPKLPGLLELEYKGFYVRGFFVTKKKYAVIDDEGKITTRGLEIVRRDWSEIAKETQARVLEAILKGGDVEEAVRIVKEVTEKLSKYEVPPEKLVIHEQITRDLRDYKATGPHVAVAKRLAARGVKIRPGTVISYIVLKGSGRIGDRAIPADEFDPTKHRYDAEYYIENQVLPAVERILKAFGYRKEDLRYQKTKQVGLGAWLKVKGKK

SEQ ID NO: 3 - protein, 775 aa, synthetic construct
MILDTDYITENGKPVIRVFKKENGEFKIEYDRTFEPYFYALLKDDSAIEDVKKVTAKRHGTVVKVKRAEKVQKKFLGRPIEVWKLYFNHPQDVPAIRDRIRAHPAVVDIYEYDIPFAKRYLIDKGLIPAEGDEELTMLAFAIATLYHEGEEFGTGPILMISYADGSEARVITWKKIDLPYVDVVSTEKEMIKRFLRVVREKDPDVLITYNGDNFDFAYLKKRSEELGIKFTLGRDGSEPKIQRMGDYFAVEVKGRIHFDLYPVIRRTINLPTYTLEAVYEAVFGKPKEKVYAEEIAQAWESGEGLERVARYSMEDAKVTYELGREFFPMEAQLSRLIGQSLWDVSRSSKGNLVEWFLLRKAYKRNELAPNKPDERELARRRGGYAGGYVKEPERGLWDNIVYLDFRSAAISIIITHNVSPDTLNREGCKEYDVAPEVGHKFCKDFPGFIPSLLGDLLEERQKIKRKMKATVDPLEKKLLDYRQRVIKILANSFYGYGGYAKARWYCKECAESVTAMGREYIEMVIRELEEKFGFKVLYADTDGLHATIPGADAETVKKKAKEFLKYINPKLPGLLELEYKGFYVRGFFVTKKKYAVIDDEGKITTRGLEIVRRDWSEIAKETQARVLEAILKGGDVEEAVRIVKEVTEKLSKYEVPPEKLVIHEQITRDLRDYKATGPHVAVAKRLAARGVKIRPGTVISYIVLKGSGRIGDRAIPADEFDPTKHRYDAEYYIENQVLPAVERILKAFGYRKEDLRYQKTKQVGLGAWLKVKGKK

SEQ ID NO: 4 - protein, 775 aa, synthetic construct
MILDTDYITENGKPVIRVFKKENGEFKIEYDRTFEPYFYALLKDDSAIEDVKKVTAKRHGTVVKVKRAEKVQKKFLGRPIEVWKLYFNHPQDVPAIRDRIRAHPAVVDIYEYDIPFAKRYLIDKGLIPAEGDEELTMLAFAIATLYHEGEEFGTGPILMISYADGSEARVITWKKIDLPYVDVVSTEKEMIKRFLRVVREKDPDVLITYNGDNFDFAYLKKRSEELGIKFTLGRDGSEPKIQRMGDYFAVEVKGRIHFDLYPVIRRTINLPTYTLEAVYEAVFGKPKEKVYAEEIAQAWESGEGLERVARYSMEDAKVTYELGREFFPMEAQLSRLIGQSLWDVSRSSKGNLVEWFLLRKAYKRNELAPNKPDERELARRRGGYAGGYVKEPERGLWDNIVYLDFRSSAVSIIITHNVSPDTLNREGCKEYDVAPEVGHKFCKDFPGFIPSLLGDLLEERQKIKRKMKATVDPLEKKLLDYRQRVIKILANSFYGYGGYAKARWYCKECAESVTAWGREYIEMVIRELEEKFGFKVLYADTDGLHATIPGADAETVKKKAKEFLKYINPKLPGLLELEYKGFYVRGFFVTKKKYAVIDDEGKITTRGLEIVRRDWSEIAKETQARVLEAILKGGDVEEAVRIVKEVTEKLSKYEVPPEKLVIHEQITRDLRDYKATGPHVAVAKRLAARGVKIRPGTVISYIVLKGSGRIGDRAIPADEFDPTKHRYDAEYYIENQVLPAVERILKAFGYRKEDLRYQKTKQVGLGAWLKVKGKK

SEQ ID NO: 5 - protein, 775 aa, synthetic construct
MILDTDYITENGKPVIRVFKKENGEFKIEYDRTFEPYFYALLKDDSAIEDVKKVTAKRHGTVVKVKRAEKVQKKFLGRPIEVWKLYFNHPQDVPAIRDRIRAHPAVVDIYEYDIPFAKRYLIDKGLIPAEGDEELTMLAFAIATLYHEGEEFGTGPILMISYADGSEARVITWKKIDLPYVDVVSTEKEMIKRFLRVVREKDPDVLITYNGDNFDFAYLKKRSEELGIKFTLGRDGSEPKIQRMGDYFAVEVKGRIHFDLYPVIRRTINLPTYTLEAVYEAVFGKPKEKVYAEEIAQAWESGEGLERVARYSMEDAKVTYELGREFFPMEAQLSRLIGQSLWDVSRSSKGNLVEWFLLRKAYKRNELAPNKPDERELARRRGGYAGGYVKEPERGLWDNIVYLDFRSSAISIIITHNVSPDTLNREGCKEYDVAPEVGHKFCKDFPGFIPSLLGDLLEERQKIKRKMKATVDPLEKKLLDYRQRVIKILANSFYGYGGYAKARWYCKECAESVTAWGREYIEMVIRELEEKFGFKVLYADTDGLHATIPGADAETVKKKAKEFLKYINPKLPGLLELEYKGFYVRGFFVTKKKYAVIDDEGKITTRGLEIVRRDWSEIAKETQARVLEAILKGGDVEEAVRIVKEVTEKLSKYEVPPEKLVIHEQITRDLRDYKATGPHVAVAKRLAARGVKIRPGTVISYIVLKGSGRIGDRAIPADEFDPTKHRYDAEYYIENQVLPAVERILKAFGYRKEDLRYQKTKQVGLGAWLKVKGKK

SEQ ID NO: 6 - protein, 775 aa, synthetic construct
MILDTDYITENGKPVIRVFKKENGEFKIEYDRTFEPYFYALLKDDSAIEDVKKVTAKRHGTVVKVKRAEKVQKKFLGRPIEVWKLYFNHPQDVPAIRDRIRAHPAVVDIYEYDIPFAKRYLIDKGLIPAEGDEELTMLAFAIATLYHEGEEFGTGPILMISYADGSEARVITWKKIDLPYVDVVSTEKEMIKRFLRVVREKDPDVLITYNGDNFDFAYLKKRSEELGIKFTLGRDGSEPKIQRMGDYFAVEVKGRIHFDLYPVIRRTINLPTYTLEAVYEAVFGKPKEKVYAEEIAQAWESGEGLERVARYSMEDAKVTYELGREFFPMEAQLSRLIGQSLWDVSRSSKGNLVEWFLLRKAYKRNELAPNKPDERELARRRGGYAGGYVKEPERGLWDNIVYLDMRSAAISIIITHNVSPDTLNREGCKEYDVAPEVGHKFCKDFPGFIPSLLGDLLEERQKIKRKMKATVDPLEKKLLDYRQRGIKILANSFYGYGGYAKARWYCKECAESVTAWGREYIEMVIRELEEKFGFKVLYADTDGLHATIPGADAETVKKKAKEFLKYINPKLPGLLELEYEGFYVRGFFVTKKKYAVIDDEGKITTRGLEIVRRDWSEIAKETQARVLEAILKGGDVEEAVRIVKEVTEKLSKYEVPPEKLVIHEQITRDLRDYKATGPHVAVAKRLAARGVKIRPGTVISYIVLKGSGRIGDRAIPADEFDPTKHRYDAEYYIENQVLPAVERILKAFGYRKEDLRYQKTKQVGLGAWLKVKGKK

SEQ ID NO: 7 - protein, 775 aa, synthetic construct
MILDTDYITENGKPVIRVFKKENGEFKIEYDRTFEPYFYALLKDDSAIEDVKKVTAKRHGTVVKVKRAEKVQKKFLGRPIEVWKLYFNHPQDVPAIRDRIRAHPAVVDIYEYDIPFAKRYLIDKGLIPAEGDEELTMLAFAIATLYHEGEEFGTGPILMISYADGSEARVITWKKIDLPYVDVVSTEKEMIKRFLRVVREKDPDVLITYNGDNFDFAYLKKRSEELGIKFTLGRDGSEPKIQRMGDYFAVEVKGRIHFDLYPVIRRTINLPTYTLEAVYEAVFGKPKEKVYAEEIAQAWESGEGLERVARYSMEDAKVTYELGREFFPMEAQLSRLIGQSLWDVSRSSKGNLVEWFLLRKAYKRNELAPNKPDERELARRRGGYAGGYVKEPERGLWDNIVYLDMRSSAVSIIITHNVSPDTLNREGCKEYDVAPEVGHKFCKDFPGFIPSLLGDLLEERQKIKRKMKATVDPLEKKLLDYRQRGIKILANSFYGYGGYAKARWYCKECAESVTAWGREYIEMVIRELEEKFGFKVLYADTDGLHATIPGADAETVKKKAKEFLKYINPKLPGLLELEYEGFYVRGFFVTKKKYAVIDDEGKITTRGLEIVRRDWSEIAKETQARVLEAILKGGDVEEAVRIVKEVTEKLSKYEVPPEKLVIHEQITRDLRDYKATGPHVAVAKRLAARGVKIRPGTVISYIVLKGSGRIGDRAIPADEFDPTKHRYDAEYYIENQVLPAVERILKAFGYRKEDLRYQKTKQVGLGAWLKVKGKK

SEQ ID NO: 8 - protein, 775 aa, synthetic construct
MILDTDYITENGKPVIRVFKKENGEFKIEYDRTFEPYFYALLKDDSAIEDVKKVTAKRHGTVVKVKRAEKVQKKFLGRPIEVWKLYFNHPQDVPAIRDRIRAHPAVVDIYEYDIPFAKRYLIDKGLIPAEGDEELTMLAFAIATLYHEGEEFGTGPILMISYADGSEARVITWKKIDLPYVDVVSTEKEMIKRFLRVVREKDPDVLITYNGDNFDFAYLKKRSEELGIKFTLGRDGSEPKIQRMGDYFAVEVKGRIHFDLYPVIRRTINLPTYTLEAVYEAVFGKPKEKVYAEEIAQAWESGEGLERVARYSMEDAKVTYELGREFFPMEAQLSRLIGQSLWDVSRSSKGNLVEWFLLRKAYKRNELAPNKPDERELARRRGGYAGGYVKEPERGLWDNIVYLDMRSSAISIIITHNVSPDTLNREGCKEYDVAPEVGHKFCKDFPGFIPSLLGDLLEERQKIKRKMKATVDPLEKKLLDYRQRGIKILANSFYGYGGYAKARWYCKECAESVTAWGREYIEMVIRELEEKFGFKVLYADTDGLHATIPGADAETVKKKAKEFLKYINPKLPGLLELEYEGFYVRGFFVTKKKYAVIDDEGKITTRGLEIVRRDWSEIAKETQARVLEAILKGGDVEEAVRIVKEVTEKLSKYEVPPEKLVIHEQITRDLRDYKATGPHVAVAKRLAARGVKIRPGTVISYIVLKGSGRIGDRAIPADEFDPTKHRYDAEYYIENQVLPAVERILKAFGYRKEDLRYQKTKQVGLGAWLKVKGKK

SEQ ID NO: 9 - protein, 775 aa, Thermococcus sp.
MILDTDYITENGKPVIRVFKKENGEFKIEYDRTFEPYFYALLKDDSAIEDVKKVTAKRHGTVVKVKRAEKVQKKFLGRPIEVWKLYFNHPQDVPAIRDRIRAHPAVVDIYEYDIPFAKRYLIDKGLIPMEGDEELTMLAFDIETLYHEGEEFGTGPILMISYADGSEARVITWKKIDLPYVDVVSTEKEMIKRFLRVVREKDPDVLITYNGDNFDFAYLKKRCEELGIKFTLGRDGSEPKIQRMGDRFAVEVKGRIHFDLYPVIRRTINLPTYTLEAVYEAVFGKPKEKVYAEEIAQAWESGEGLERVARYSMEDAKVTYELGREFFPMEAQLSRLIGQSLWDVSRSSTGNLVEWFLLRKAYKRNELAPNKPDERELARRRGGYAGGYVKEPERGLWDNIVYLDFRSLYPSIIITHNVSPDTLNREGCKEYDVAPEVGHKFCKDFPGFIPSLLGDLLEERQKIKRKMKATVDPLEKKLLDYRQRAIKILANSFYGYYGYAKARWYCKECAESVTAWGREYIEMVIRELEEKFGFKVLYADTDGLHATIPGADAETVKKKAKEFLKYINPKLPGLLELEYEGFYVRGFFVTKKKYAVIDEEGKITTRGLEIVRRDWSEIAKETQARVLEAILKHGDVEEAVRIVKEVTEKLSKYEVPPEKLVIHEQITRDLRDYKATGPHVAVAKRLAARGVKIRPGTVISYIVLKGSGRIGDRAIPADEFDPTKHRYDAEYYIENQVLPAVERILKAFGYRKEDLRYQKTKQVGLGAWLKVKGKK

SEQ ID NO: 10 - protein, 774 aa, synthetic construct
MILDTDYITKDGKPIIRIFKKENGEFKIELDPHFQPYIYALLKDDSAIEEIKAIKGERHGKTVRVLDAVKVRKKFLGREVEVWKLIFEHPQDVPAMRGKIREHPAVVDIYEYDIPFAKRYLIDKGLIPMEGDEELKLLAFAIATFYHEGDEFGKGEIIMISYADEEEARVITWKNIDLPYVDVVSNEREMIKRFVQVVKEKDPDVIITYNGDNFDLPYLIKRAEKLGVRLVLGRDKEHPEPKIQRMGDSFAVEIKGRIHFDLFPVVRRTINLPTYTLEAVYEAVLGKTKSKLGAEEIAAIWETEESMKKLAQYSMEDARATYELGKEFFPMEAELAKLIGQSVWDVSRSSTGNLVEWYLLRVAYARNELAPNKPDEEEYKRRLRTTYLGGYVKEPEKGLWENIIYLDFRSLYPSIIVTHNVSPDTLEKEGCKNYDVAPIVGYRFCKDFPGFIPSILGDLIAMRQDIKKKMKSTIDPIEKKMLDYRQRAIKLLANSYYGYMGYPKARWYSKECAESVTAWGRHYIEMTIREIEEKFGFKVLYADTDGFYATIPGEKPELIKKKAKEFLNYINSKLPGLLELEYEGFYLRGFFVTKKRYAVIDEEGRITTRGLEVVRRDWSEIAKETQAKVLEAILKEGSVEKAVEVVRDVVEKIAKYRVPLEKLVIHEQITRDLKDYKAIGPHVAIAKRLAARGIKVKPGTIISYIVLKGSGKISDRVILLTEYDPRKHKYDPDYYIENQVLPAVLRILEAFGYRKEDLRYQSSKQTGLDAWLKR

SEQ ID NO: 11 - protein, 777 aa, synthetic construct
MILDADYITEDGKPIIRIFKKENGEFKVEYDRNFRPYIYALLKDDSQIDEVRKITAERHGKIVRIIDAEKVRKKFLGRPIEVWRLYFEHPQDVPAIRDKIREHSAVIDIFEYDIPFAKRYLIDKGLIPMEGDEELKLLAFDIETLYHEGEEFAKGPIIMISYADEEEAKVITWKKIDLPYVEVVSSEREMIKRFLKVIREKDPDVIITYNGDSFDLPYLVKRAEKLGIKLPLGRDGSEPKMQRLGDMTAVEIKGRIHFDLYHVIRRTINLPTYTLEAVYEAIFGKPKEKVYAHEIAEAWETGKGLERVAKYSMEDAKVTYELGREFFPMEAQLSRLVGQPLWDVSRSSTGNLVEWYLLRKAYERNELAPNKPDEREYERRLRESYAGGYVKEPEKGLWEGLVSLDFRSLYPSIIITHNVSPDTLNREGCREYDVAPEVGHKFCKDFPGFIPSLLKRLLDERQEIKRKMKASKDPIEKKMLDYRQRAIKILANSYYGYYGYAKARWYCKECAESVTAWGREYIEFVRKELEEKFGFKVLYIDTDGLYATIPGAKPEEIKKKALEFVDYINAKLPGLLELEYEGFYVRGFFVTKKKYALIDEEGKIITRGLEIVRRDWSEIAKETQAKVLEAILKHGNVEEAVKIVKEVTEKLSKYEIPPEKLVIYEQITRPLHEYKAIGPHVAVAKRLAARGVKVRPGMVIGYIVLRGDGPISKRAILAEEFDLRKHKYDAEYYIENQVLPAVLRILEAFGYRKEDLRWQKTKQTGLTAWLNIKKKGS

SEQ ID NO: 12 - protein, 775 aa, Thermococcus waiotapuensis
MILDADYITEDGKPVIRVFKKEKGEFKINYDRDFEPYIYALLKDDSAIEDIKKITAERHGTTVRVTRAERVKKKFLGRPVEVWKLYFTHPQDVPAIRDKIREHPAVVDIYEYDIPFAKRYLIDKGLIPMEGNEELRMLAFDIETLYHEGEEFGEGPILMISYADEEGARVITWKNIDLPYVESVSTEKEMIKRFLKVIQEKDPDVLITYNGDNFDFAYLKKRSETLGVKFILGRDGSEPKIQRMGDRFAVEVKGRIHFDLYPVIRRTINLPTYTLETVYEAIFGQPKEKVYAEEIAQAWESGEGLERVARYSMEDAKATYELGKEFFPMEAQLSRLVGQSLWDVSRSSTGNLVEWFLLRKAYERNELAPNKPDERELARRAESYAGGYVKEPEKGLWENIVYLDYKSLYPSIIITHNVSPDTLNREGCREYDVAPQVGHRFCKDFPGFIPSLLGDLLEERQKVKKKMKATVDPIERKLLDYRQRAIKILANSYYGYYGYANARWYCRECAESVTAWGRQYIETTMREIEEKFGFKVLYADTDGFFATIPGADAETVKKKTKEFLNYINPRLPGLLELEYEGFYRRGFFVTKKKYAVIDEEDKITTRGLEIVRRDWSEIAKETQARVLEAILKHGDVEEAVRIVKEVTEKLSRYEVPPEKLVIYEQITRNLRDYRATGPHVAVAKRLAARGIKIRPGTVISYIVLKGPGRVGDRAIPFDEFDPAKHRYDAEYYIENQVLPAVERILRAFGYRKEDLRYQKTKQAGLGAWLKPKTGS

SEQ ID NO: 13 - protein, 774 aa, Thermococcus kodakarensis
MILDTDYITEDGKPVIRIFKKENGEFKIEYDRTFEPYFYALLKDDSAIEEVKKITAERHGTVVTVKRVEKVQKKFLGRPVEVWKLYFTHPQDVPAIRDKIREHPAVIDIYEYDIPFAKRYLIDKGLVPMEGDEELKMLAFAIATLYHEGEEFAEGPILMISYADEEGARVITWKNVDLPYVDVVSTEREMIKRFLRVVKEKDPDVLITYNGDNFDFAYLKKRCEKLGINFALGRDGSEPKIQRMGDRFAVEVKGRIHFDLYPVIRRTINLPTYTLEAVYEAVFGQPKEKVYAEEITTAWETGENLERVARYSMEDAKVTYELGKEFLPMEAQLSRLIGQSLWDVSRSSTGNLVEWFLLRKAYERNELAPNKPDEKELARRRQSYEGGYVKEPERGLWENIVYLDFRSLYPSIIITHNVSPDTLNREGCKEYDVAPQVGHRFCKDFPGFIPSLLGDLLEERQKIKKKMKATIDPIERKLLDYRQRAIKILANSYYGYYGYARARWYCKECAESVTAWGREYITMTIKEIEEKYGFKVIYSDTDGFFATIPGADAETVKKKAMEFLKYINAKLPGALELEYEGFYKRGFFVTKKKYAVIDEEGKITTRGLEIVRRDWSEIAKETQARVLEALLKDGDVEKAVRIVKEVTEKLSKYEVPPEKLVIHEQITRDLKDYKATGPHVAVAKRLAARGVKIRPGTVISYIVLKGSGRIGDRAIPFDEFDPTKHKYDAEYYIENQVLPAVERILRAFGYRKEDLRYQKTRQVGLSAWLKPKGT

SEQ ID NO: 14 - protein, 775 aa, Pyrococcus furiosus
MILDVDYITEEGKPVIRLFKKENGKFKIEHDRTFRPYIYALLRDDSKIEEVKKITGERHGKIVRIVDVEKVEKKFLGKPITVWKLYLEHPQDVPTIREKVREHPAVVDIFEYDIPFAKRYLIDKGLIPMEGEEELKILAFAIATLYHEGEEFGKGPIIMISYADENEAKVITWKNIDLPYVEVVSSEREMIKRFLRIIREKDPDIIVTYNGDSFDFPYLAKRAEKLGIKLTIGRDGSEPKMQRIGDMTAVEVKGRIHFDLYHVITRTINLPTYTLEAVYEAIFGKPKEKVYADEIAKAWESGENLERVAKYSMEDAKATYELGKEFLPMEIQLSRLVGQPLWDVSRSSTGNLVEWFLLRKAYERNEVAPNKPSEEEYQRRLRESYTGGFVKEPEKGLWENIVYLDFRALYPSIIITHNVSPDTLNLEGCKNYDIAPQVGHKFCKDIPGFIPSLLGHLLEERQKIKTKMKETQDPIEKILLDYRQKAIKLLANSFYGYYGYAKARWYCKECAESVTAWGRKYIELVWKELEEKFGFKVLYIDTDGLYATIPGGESEEIKKKALEFVKYINSKLPGLLELEYEGFYKRGFFVTKKRYAVIDEEGKVITRGLEIVRRDWSEIAKETQARVLETILKHGDVEEAVRIVKEVIQKLANYEIPPEKLAIYEQITRPLHEYKAIGPHVAVAKKLAAKGVKIKPGMVIGYIVLRGDGPISNRAILAEEYDPKKHKYDAEYYIENQVLPAVLRILEGFGYRKEDLRYQKTRQVGLTSWLNIKKS

SEQ ID NO: 15 - protein, 773 aa, Pyrococcus abyssi
MIIDADYITEDGKPIIRIFKKEKGEFKVEYDRTFRPYIYALLKDDSAIDEVKKITAERHGKIVRITEVEKVQKKFLGRPIEVWKLYLEHPQDVPAIREKIREHPAVVDIFEYDIPFAKRYLIDKGLTPMEGNEELTFLAVDIETLYHEGEEFGKGPIIMISYADEEGAKVITWKSIDLPYVEVVSSEREMIKRLVKVIREKDPDVIITYNGDNFDFPYLLKRAEKLGIKLPLGRDNSEPKMQRMGDSLAVEIKGRIHFDLFPVIRRTINLPTYTLEAVYEAIFGKSKEKVYAHEIAEAWETGKGLERVAKYSMEDAKVTFELGKEFFPMEAQLARLVGQPVWDVSRSSTGNLVEWFLLRKAYERNELAPNKPDEREYERRLRESYEGGYVKEPEKGLWEGIVSLDFRSLYPSIIITHNVSPDTLNRENCKEYDVAPQVGHRFCKDFPGFIPSLLGNLLEERQKIKKRMKESKDPVEKKLLDYRQRAIKILANSYYGYYGYAKARWYCKECAESVTAWGRQYIDLVRRELESRGFKVLYIDTDGLYATIPGAKHEEIKEKALKFVEYINSKLPGLLELEYEGFYARGFFVTKKKYALIDEEGKIVTRGLEIVRRDWSEIAKETQAKVLEAILKHGNVDEAVKIVKEVTEKLSKYEIPPEKLVIYEQITRPLSEYKAIGPHVAVAKRLAAKGVKVKPGMVIGYIVLRGDGPISKRAIAIEEFDPKKHKYDAEYYIENQVLPAVERILRAFGYRKEDLKYQKTKQVGLGAWLKFGS

SEQ ID NO: 16 - protein, 775 aa, synthetic construct
MILDTDYITENGKPVIRVFKKENGEFKIEYDRTFEPYFYALLKDDSAIEDVKKVTAKRHGTVVKVKRAEKVQKKFLGRPIEVWKLYFNHPQDVPAIRDRIRAHPAVVDIYEYDIPFAKRYLIDKGLIPAEGDEELTMLAFAIATLYHEGEEFGTGPILMISYADGSEARVITWKKIDLPYVDVVSTEKEMIKRFLRVVREKDPDVLITYNGDNFDFAYLKKRSEELGIKFTLGRDGSEPKIQRMGDYFAVEVKGRIHFDLYPVIRRTINLPTYTLEAVYEAVFGKPKEKVYAEEIAQAWESGEGLERVARYSMEDAKVTYELGREFFPMEAQLSRLIGQSLWDVSRSSKGNLVEWFLLRKAYKRNELAPNKPDERELARRRGGYAGGYVKEPERGLWDNIVYLDFRSAAISIIITHNVSPDTLNREGCKEYDVAPEVGHKFCKDFPGFIPSLLGDLLEERQKIKRKMKATVDPLEKKLLDYRQRVIKILANSFYGYGGYAKARWYCKECAESVTAWGREYIEMVIRELEEKFGFKVLYADTDGLHATIPGADAETVKKKAKEFLKYIQPKLPGLLELEYEGFYVRGFFVTKKKYAVIDDEGKITTRGLEIVRRDWSEIAKETQARVLEAILKGGDVEEAVRIVKEVTEKLSKYEVPPEKLVIHEQITRDLRDYKATGPHVAVAKRLAARGVKIRPGTVISYIVLKGSGRIGDRAIPADEFDPTKHRYDAEYYIENQVLPAVERILKAFGYRKEDLRYQKTKQVGLGAWLKVKGKK

SEQ ID NO: 17 - protein, 775 aa, synthetic construct
MILDTDYITENGKPVIRVFKKENGEFKIEYDRTFEPYFYALLKDDSAIEDVKKVTAKRHGTVVKVKRAEKVQKKFLGRPIEVWKLYFNHPQDVPAIRDRIRAHPAVVDIYEYDIPFAKRYLIDKGLIPAEGDEELTMLAFAIATLYHEGEEFGTGPILMISYADGSEARVITWKKIDLPYVDVVSTEKEMIKRFLRVVREKDPDVLITYNGDNFDFAYLKKRSEELGIKFTLGRDGSEPKIQRMGDYFAVEVKGRIHFDLYPVIRRTINLPTYTLEAVYEAVFGKPKEKVYAEEIAQAWESGEGLERVARYSMEDAKVTYELGREFFPMEAQLSRLIGQSLWDVSRSSKGNLVEWFLLRKAYKRNELAPNKPDERELARRRGGYAGGYVKEPERGLWDNIVYLDFRSAAISIIITHNVSPDTLNREGCKEYDVAPEVGHKFCKDFPGFIPSLLGDLLEERQKIKRKMKATVDPLEKKLLDYRQRVIKILANSFYGYGGYAKARWYCKECAESVTAWGREYIEMVIRELEEKFGFKVLYADTDGLHATIPGADAETVKKKAKEFLKYINPKLPGLLELEYRGFYVRGFFVTKKKYAVIDDEGKITTRGLEIVRRDWSEIAKETQARVLEAILKGGDVEEAVRIVKEVTEKLSKYEVPPEKLVIHEQITRDLRDYKATGPHVAVAKRLAARGVKIRPGTVISYIVLKGSGRIGDRAIPADEFDPTKHRYDAEYYIENQVLPAVERILKAFGYRKEDLRYQKTKQVGLGAWLKVKGKK

SEQ ID NO: 18 - protein, 775 aa, synthetic construct
MILDTDYITENGKPVIRVFKKENGEFKIEYDRTFEPYFYALLKDDSAIEDVKKVTAKRHGTVVKVKRAEKVQKKFLGRPIEVWKLYFNHPQDVPAIRDRIRAHPAVVDIYEYDIPFAKRYLIDKGLIPAEGDEELTMLAFAIATLYHEGEEFGTGPILMISYADGSEARVITWKKIDLPYVDVVSTEKEMIKRFLRVVREKDPDVLITYNGDNFDFAYLKKRSEELGIKFTLGRDGSEPKIQRMGDYFAVEVKGRIHFDLYPVIRRTINLPTYTLEAVYEAVFGKPKEKVYAEEIAQAWESGEGLERVARYSMEDAKVTYELGREFFPMEAQLSRLIGQSLWDVSRSSKGNLVEWFLLRKAYKRNELAPNKPDERELARRRGGYAGGYVKEPERGLWDNIVYLDFRSAAISIIITHNVSPDTLNREGCKEYDVAPEVGHKFCKDFPGFIPSLLGDLLEERQKIKRKMKATVDPLEKKLLDYRQRVIKILANSFYGYGGYAKARWYCKECAESVTAWGREYIEMVIRELEEKFGFKVLYADTDGLHATIPGADAETVKKKAKEFLKYINPKFPGLLELEYKGFYVRGFFVTKKKYAVIDDEGKITTRGLEIVRRDWSEIAKETQARVLEAILKGGDVEEAVRIVKEVTEKLSKYEVPPEKLVIHEQITRDLRDYKATGPHVAVAKRLAARGVKIRPGTVISYIVLKGSGRIGDRAIPADEFDPTKHRYDAEYYIENQVLPAVERILKAFGYRKEDLRYQKTKQVGLGAWLKVKGKK

SEQ ID NO: 19 - protein, 775 aa, synthetic construct
MILDTDYITENGKPVIRVFKKENGEFKIEYDRTFEPYFYALLKDDSAIEDVKKVTAKRHGTVVKVKRAEKVQKKFLGRPIEVWKLYFNHPQDVPAIRDRIRAHPAVVDIYEYDIPFAKRYLIDKGLIPAEGDEELTMLAFAIATLYHEGEEFGTGPILMISYADGSEARVITWKKIDLPYVDVVSTEKEMIKRFLRVVREKDPDVLITYNGDNFDFAYLKKRSEELGIKFTLGRDGSEPKIQRMGDYFAVEVKGRIHFDLYPVIRRTINLPTYTLEAVYEAVFGKPKEKVYAEEIAQAWESGEGLERVARYSMEDAKVTYELGREFFPMEAQLSRLIGQSLWDVSRSSKGNLVEWFLLRKAYKRNELAPNKPDERELARRRGGYAGGYVKEPERGLWDNIVYLDFRSAAISIIITHNVSPDTLNREGCKEYDVAPEVGHKFCKDFPGFIPSLLGDLLEERQKIKRKMKATVDPLEKKLLDYRQRTIKILANSFYGYGGYAKARWYCKECAESVTAWGREYIEMVIRELEEKFGFKVLYADTDGLHATIPGADAETVKKKAKEFLKYINPKLPGLLELEYKGFYVRGFFVTKKKYAVIDDEGKITTRGLEIVRRDWSEIAKETQARVLEAILKGGDVEEAVRIVKEVTEKLSKYEVPPEKLVIHEQITRDLRDYKATGPHVAVAKRLAARGVKIRPGTVISYIVLKGSGRIGDRAIPADEFDPTKHRYDAEYYIENQVLPAVERILKAFGYRKEDLRYQKTKQVGLGAWLKVKGKK

SEQ ID NO: 20 - protein, 775 aa, synthetic construct
MILDTDYITENGKPVIRVFKKENGEFKIEYDRTFEPYFYALLKDDSAIEDVKKVTAKRHGTVVKVKRAEKVQKKFLGRPIEVWKLYFNHPQDVPAIRDRIRAHPAVVDIYEYDIPFAKRYLIDKGLIPAEGDEELTMLAFAIATLYHEGEEFGTGPILMISYADGSEARVITWKKIDLPYVDVVSTEKEMIKRFLRVVREKDPDVLITYNGDNFDFAYLKKRSEELGIKFTLGRDGSEPKIQRMGDYFAVEVKGRIHFDLYPVIRRTINLPTYTLEAVYEAVFGKPKEKVYAEEIAQAWESGEGLERVARYSMEDAKVTYELGREFFPMEAQLSRLIGQSLWDVSRSSKGNLVEWFLLRKAYKRNELAPNKPDERELARRRGGYAGGYVKEPERGLWDNIVYLDFRSAAISIIITHNVSPDTLNREGCKEYDVAPEVGHKFCKDFPGFIPSLLGDLLEERQKIKRKMKATVDPLEKKLLDYRQRVIKILANSFYGYGGYAKARWYCKECAESVTALGREYIEMVIRELEEKFGFKVLYADTDGLHATIPGADAETVKKKAKEFLKYINPKLPGLLELEYKGFYVRGFFVTKKKYAVIDDEGKITTRGLEIVRRDWSEIAKETQARVLEAILKGGDVEEAVRIVKEVTEKLSKYEVPPEKLVIHEQITRDLRDYKATGPHVAVAKRLAARGVKIRPGTVISYIVLKGSGRIGDRAIPADEFDPTKHRYDAEYYIENQVLPAVERILKAFGYRKEDLRYQKTKQVGLGAWLKVKGKK

SEQ ID NO: 21 - protein, 775 aa, synthetic construct
MILDTDYITENGKPVIRVFKKENGEFKIEYDRTFEPYFYALLKDDSAIEDVKKVTAKRHGTVVKVKRAEKVQKKFLGRPIEVWKLYFNHPQDVPAIRDRIRAHPAVVDIYEYDIPFAKRYLIDKGLIPAEGDEELTMLAFAIATLYHEGEEFGTGPILMISYADGSEARVITWKKIDLPYVDVVSTEKEMIKRFLRVVREKDPDVLITYNGDNFDFAYLKKRSEELGIKFTLGRDGSEPKIQRMGDYFAVEVKGRIHFDLYPVIRRTINLPTYTLEAVYEAVFGKPKEKVYAEEIAQAWESGEGLERVARYSMEDAKVTYELGREFFPMEAQLSRLIGQSLWDVSRSSKGNLVEWFLLRKAYKRNELAPNKPDERELARRRGGYAGGYVKEPERGLWDNIVYLDFRSAAVSIIITHNVSPDTLNREGCKEYDVAPEVGHKFCKDFPGFIPSLLGDLLEERQKIKRKMKATVDPLEKKLLDYRQRVIKILANSFYGYGGYAKARWYCKECAESVTAWGREYIEMVIRELEEKFGFKVLYADTDGLHATIPGADAETVKKKAKEFLKYINPKLPGLLELEYKGFYVRGFFVTKKKYAVIDDEGKITTRGLEIVRRDWSEIAKETQARVLEAILKGGDVEEAVRIVKEVTEKLSKYEVPPEKLVIHEQITRDLRDYKATGPHVAVAKRLAARGVKIRPGTVISYIVLKGSGRIGDRAIPADEFDPTKHRYDAEYYIENQVLPAVERILKAFGYRKEDLRYQKTKQVGLGAWLKVKGKK

SEQ ID NO: 22 - protein, 775 aa, synthetic construct
MILDTDYITENGKPVIRVFKKENGEFKIEYDRTFEPYFYALLKDDSAIEDVKKVTAKRHGTVVKVKRAEKVQKKFLGRPIEVWKLYFNHPQDVPAIRDRIRAHPAVVDIYEYDIPFAKRYLIDKGLIPAEGDEELTMLAFAIATLYHEGEEFGTGPILMISYADGSEARVITWKKIDLPYVDVVSTEKEMIKRFLRVVREKDPDVLITYNGDNFDFAYLKKRSEELGIKFTLGRDGSEPKIQRMGDYFAVEVKGRIHFDLYPVIRRTINLPTYTLEAVYEAVFGKPKEKVYAEEIAQAWESGEGLERVARYSMEDAKVTYELGREFFPMEAQLSRLIGQSLWDVSRSSKGNLVEWFLLRKAYKRNELAPNKPDERELARRRGGYAGGYVKEPERGLWDNIVYLDFRSAAISIIITHNVSPDTLNREGCKEYDVAPEVGHKFCKDFPGFIPSLLGDLLEERQKIKRKMKATVDPLEKKLLDYRQRVIKILANSFYGYGGYAKARWYCKECAESVTAWGREYIEMVIRELEEKFGFKVLYADTDGLHATIPGADAETVKKKAKEFLKYINPKMPGLLELEYQGFYVRGFFVTKKKYAVIDDEGKITTRGLEIVRRDWSEIAKETQARVLEAILKGGDVEEAVRIVKEVTEKLSKYEVPPEKLVIHEQITRDLRDYKATGPHVAVAKRLAARGVKIRPGTVISYIVLKGSGRIGDRAIPADEFDPTKHRYDAEYYIENQVLPAVERILKAFGYRKEDLRYQKTKQVGLGAWLKVKGKK

SEQ ID NO: 23 - protein, 775 aa, synthetic construct
MILDTDYITENGKPVIRVFKKENGEFKIEYDRTFEPYFYALLKDDSAIEDVKKVTAKRHGTVVKVKRAEKVQKKFLGRPIEVWKLYFNHPQDVPAIRDRIRAHPAVVDIYEYDIPFAKRYLIDKGLIPAEGDEELTMLAFAIATLYHEGEEFGTGPILMISYADGSEARVITWKKIDLPYVDVVSTEKEMIKRFLRVVREKDPDVLITYNGDNFDFAYLKKRSEELGIKFTLGRDGSEPKIQRMGDYFAVEVKGRIHFDLYPVIRRTINLPTYTLEAVYEAVFGKPKEKVYAEEIAQAWESGEGLERVARYSMEDAKVTYELGREFFPMEAQLSRLIGQSLWDVSRSSKGNLVEWFLLRKAYKRNELAPNKPDERELARRRGGYAGGYVKEPERGLWDNIVYLDMRSAAISIIITHNVSPDTLNREGCKEYDVAPEVGHKFCKDFPGFIPSLLGDLLEERQKIKRKMKATVDPLEKKLLDYRQRVIKILANSFYGYGGYAKARWYCKECAESVTAWGREYIEMVIRELEEKFGFKVLYADTDGLHATIPGADAETVKKKAKEFLKYINPKLPGLLELEYEGFYVRGFFVTKKKYAVIDDEGKITTRGLEIVRRDWSEIAKETQARVLEAILKGGDVEEAVRIVKEVTEKLSKYEVPPEKLVIHEQITRDLRDYKATGPHVAVAKRLAARGVKIRPGTVISYIVLKGSGRIGDRAIPADEFDPTKHRYDAEYYIENQVLPAVERILKAFGYRKEDLRYQKTKQVGLGAWLKVKGKK

SEQ ID NO: 24 - protein, 775 aa, synthetic construct
MILDTDYITENGKPVIRVFKKENGEFKIEYDRTFEPYFYALLKDDSAIEDVKKVTAKRHGTVVKVKRAEKVQKKFLGRPIEVWKLYFNHPQDVPAIRDRIRAHPAVVDIYEYDIPFAKRYLIDKGLIPAEGDEELTMLAFAIATLYHEGEEFGTGPILMISYADGSEARVITWKKIDLPYVDVVSTEKEMIKRFLRVVREKDPDVLITYNGDNFDFAYLKKRSEELGIKFTLGRDGSEPKIQRMGDYFAVEVKGRIHFDLYPVIRRTINLPTYTLEAVYEAVFGKPKEKVYAEEIAQAWESGEGLERVARYSMEDAKVTYELGREFFPMEAQLSRLIGQSLWDVSRSSKGNLVEWFLLRKAYKRNELAPNKPDERELARRRGGYAGGYVKEPERGLWDNIVYLDMRSAAVSIIITHNVSPDTLNREGCKEYDVAPEVGHKFCKDFPGFIPSLLGDLLEERQKIKRKMKATVDPLEKKLLDYRQRVIKILANSFYGYGGYAKARWYCKECAESVTAWGREYIEMVIRELEEKFGFKVLYADTDGLHATIPGADAETVKKKAKEFLKYINPKLPGLLELEYEGFYVRGFFVTKKKYAVIDDEGKITTRGLEIVRRDWSEIAKETQARVLEAILKGGDVEEAVRIVKEVTEKLSKYEVPPEKLVIHEQITRDLRDYKATGPHVAVAKRLAARGVKIRPGTVISYIVLKGSGRIGDRAIPADEFDPTKHRYDAEYYIENQVLPAVERILKAFGYRKEDLRYQKTKQVGLGAWLKVKGKK

SEQ ID NO: 25 - protein, 775 aa, synthetic construct
MILDTDYITENGKPVIRVFKKENGEFKIEYDRTFEPYFYALLKDDSAIEDVKKVTAKRHGTVVKVKRAEKVQKKFLGRPIEVWKLYFNHPQDVPAIRDRIRAHPAVVDIYEYDIPFAKRYLIDKGLIPAEGDEELTMLAFAIATLYHEGEEFGTGPILMISYADGSEARVITWKKIDLPYVDVVSTEKEMIKRFLRVVREKDPDVLITYNGDNFDFAYLKKRSEELGIKFTLGRDGSEPKIQRMGDYFAVEVKGRIHFDLYPVIRRTINLPTYTLEAVYEAVFGKPKEKVYAEEIAQAWESGEGLERVARYSMEDAKVTYELGREFFPMEAQLSRLIGQSLWDVSRSSKGNLVEWFLLRKAYKRNELAPNKPDERELARRRGGYAGGYVKEPERGLWDNIVYLDFRSAAISIIITHNVSPDTLNREGCKEYDVAPEVGHKFCKDFPGFIPSLLGDLLEERQKIKRKMKATVDPLEKKLLDYRQRVIKILANSFYGYGGYAKARWYCKECAESVTAWGREYIEMVIRELEEKFGFKVLYADTDGLHATIPGADAETVKKKAKEFLKYINPKLPGLLELEYIGFYVRGFFVTKKKYAVIDDEGKITTRGLEIVRRDWSEIAKETQARVLEAILKGGDVEEAVRIVKEVTEKLSKYEVPPEKLVIHEQITRDLRDYKATGPHVAVAKRLAARGVKIRPGTVISYIVLKGSGRIGDRAIPADEFDPTKHRYDAEYYIENQVLPAVERILKAFGYRKEDLRYQKTKQVGLGAWLKVKGKK

SEQ ID NO: 26 - protein, 775 aa, synthetic construct
MILDTDYITENGKPVIRVFKKENGEFKIEYDRTFEPYFYALLKDDSAIEDVKKVTAKRHGTVVKVKRAEKVQKKFLGRPIEVWKLYFNHPQDVPAIRDRIRAHPAVVDIYEYDIPFAKRYLIDKGLIPAEGDEELTMLAFAIATLYHEGEEFGTGPILMISYADGSEARVITWKKIDLPYVDVVSTEKEMIKRFLRVVREKDPDVLITYNGDNFDFAYLKKRSEELGIKFTLGRDGSEPKIQRMGDYFAVEVKGRIHFDLYPVIRRTINLPTYTLEAVYEAVFGKPKEKVYAEEIAQAWESGEGLERVARYSMEDAKVTYELGREFFPMEAQLSRLIGQSLWDVSRSSKGNLVEWFLLRKAYKRNELAPNKPDERELARRRGGYAGGYVKEPERGLWDNIVYLDKRSAAISIIITHNVSPDTLNREGCKEYDVAPEVGHKFCKDFPGFIPSLLGDLLEERQKIKRKMKATVDPLEKKLLDYRQRVIKILANSFYGYGGYAKARWYCKECAESVTAWGREYIEMVIRELEEKFGFKVLYADTDGLHATIPGADAETVKKKAKEFLKYINPKLPGLLELEYEGFYVRGFFVTKKKYAVIDDEGKITTRGLEIVRRDWSEIAKETQARVLEAILKGGDVEEAVRIVKEVTEKLSKYEVPPEKLVIHEQITRDLRDYKATGPHVAVAKRLAARGVKIRPGTVISYIVLKGSGRIGDRAIPADEFDPTKHRYDAEYYIENQVLPAVERILKAFGYRKEDLRYQKTKQVGLGAWLKVKGKK

SEQ ID NO: 27 - protein, 775 aa, synthetic construct
MILDTDYITENGKPVIRVFKKENGEFKIEYDRTFEPYFYALLKDDSAIEDVKKVTAKRHGTVVKVKRAEKVQKKFLGRPIEVWKLYFNHPQDVPAIRDRIRAHPAVVDIYEYDIPFAKRYLIDKGLIPAEGDEELTMLALAIATLYHEGEEFGTGPILMISYADGSEARVITWKKIDLPYVDVVSTEKEMIKRFLRVVREKDPDVLITYNGDNFDFAYLKKRSEELGIKFTLGRDGSEPKIQRMGDYFAVEVKGRIHFDLYPVIRRTINLPTYTLEAVYEAVFGKPKEKVYAEEIAQAWESGEGLERVARYSMEDAKVTYELGREFFPMEAQLSRLIGQSLWDVSRSSKGNLVEWFLLRKAYKRNELAPNKPDERELARRRGGYAGGYVKEPERGLWDNIVYLDFRLAAISIIITHNVSPDTLNREGCKEYDVAPEVGHKFCKDFPGFIPSLLGDLLEERQKIKRKMKATVDPLEKKLLDYRQRVIKILANSFYGYGGYAKARWYCKECAESVTAWGREYIEMVIRELEEKFGFKVLYADTDGLHATIPGADAETVKKKAKEFLKYINPKLPGLLELEYEGFYVRGFFVTKKKYAVIDDEGKITTRGLEIVRRDWSEIAKETQARVLEAILKGGDVEEAVRIVKEVTEKLSKYEVPPEKLVIHEQITRDLRDYKATGPHVAVAKRLAARGVKIRPGTVISYIVLKGSGRIGDRAIPADEFDPTKHRYDAEYYIENQVLPAVERILKAFGYRKEDLRYQKTKQVGLGAWLKVKGKK

SEQ ID NO: 28 - protein, 775 aa, synthetic construct
MILDTDYITENGKPVIRVFKKENGEFKIEYDRTFEPYFYALLKDDSAIEDVKKVTAKRHGTVVKVKRAEKVQKKFLGRPIEVWKLYFNHPQDVPAIRDRIRAHPAVVDIYEYDIPFAKRYLIDKGLIPAEGDEELTMLAFAIATLYHEGEEFGTGPILMISYADGSEARVITWKKIDLPYVDVVSTEKEMIKRFLRVVREKDPDVLITYNGDNFDFAYLKKRSEELGIKFTLGRDGSEPKIQRMGDYFAVEVKGRIHFDLYPVIRRTINLPTYTLEAVYEAVFGKPKEKVYAEEIAQAWESGEGLERVARYSMEDAKVTYELGREFFPMEAQLSRLIGQSLWDVSRSSKGNLVEWFLLRKAYKRNELAPNKPDERELARRRGGYAGGYVKEPERGLWDNIVYLDMRSAAISIIITHNVSPDTLNREGCKEYDVAPEVGHKFCKDFPGFIPSLLGDLLEERQKIKRKMKATVDPLEKKLLDYRQRVIKILANSFYGYGGYAKARWYCKECAESVTAWGREYIEMVIRELEEKFGFKVLYADTDGLHATIPGADAETVKKKAKEFLKYINPKLPGLLELEYRGFYVRGFFVTKKKYAVIDDEGKITTRGLEIVRRDWSEIAKETQARVLEAILKGGDVEEAVRIVKEVTEKLSKYEVPPEKLVIHEQITRDLRDYKATGPHVAVAKRLAARGVKIRPGTVISYIVLKGSGRIGDRAIPADEFDPTKHRYDAEYYIENQVLPAVERILKAFGYRKEDLRYQKTKQVGLGAWLKVKGKK

SEQ ID NO: 29 - protein, 775 aa, synthetic construct
MILDTDYITENGKPVIRVFKKENGEFKIEYDRTFEPYFYALLKDDSAIEDVKKVTAKRHGTVVKVKRAEKVQKKFLGRPIEVWKLYFNHPQDVPAIRDRIRAHPAVVDIYEYDIPFAKRYLIDKGLIPAEGDEELTMLAFAIATLYHEGEEFGTGPILMISYADGSEARVITWKKIDLPYVDVVSTEKEMIKRFLRVVREKDPDVLITYNGDNFDFAYLKKRSEELGIKFTLGRDGSEPKIQRMGDYFAVEVKGRIHFDLYPVIRRTINLPTYTLEAVYEAVFGKPKEKVYAEEIAQAWESGEGLERVARYSMEDAKVTYELGREFFPMEAQLSRLIGQSLWDVSRSSKGNLVEWFLLRKAYKRNELAPNKPDERELARRRGGYAGGYVKEPERGLWDNIVYLDMRSAAISIIITHNVSPDTLNREGCKEYDVAPEVGHKFCKDFPGFIPSLLGDLLEERQKIKRKMKATVDPLEKKLLDYRQRVIKILANSFYGYGGYAKARWYCKECAESVTAWGREYIEMVIRELEEKFGFKVLYADTDGLHATIPGADAETVKKKAKEFLKYINPKLPGLLELEYKGFYVRGFFVTKKKYAVIDDEGKITTRGLEIVRRDWSEIAKETQARVLEAILKGGDVEEAVRIVKEVTEKLSKYEVPPEKLVIHEQITRDLRDYKATGPHVAVAKRLAARGVKIRPGTVISYIVLKGSGRIGDRAIPADEFDPTKHRYDAEYYIENQVLPAVERILKAFGYRKEDLRYQKTKQVGLGAWLKVKGKK

SEQ ID NO: 30 - protein, 775 aa, synthetic construct
MILDTDYITENGKPVIRVFKKENGEFKIEYDRTFEPYFYALLKDDSAIEDVKKVTAKRHGTVVKVKRAEKVQKKFLGRPIEVWKLYFNHPQDVPAIRDRIRAHPAVVDIYEYDIPFAKRYLIDKGLIPAEGDEELTMLAFAIATLYHEGEEFGTGPILMISYADGSEARVITWKKIDLPYVDVVSTEKEMIKRFLRVVREKDPDVLITYNGDNFDFAYLKKRSEELGIKFTLGRDGSEPKIQRMGDYFAVEVKGRIHFDLYPVIRRTINLPTYTLEAVYEAVFGKPKEKVYAEEIAQAWESGEGLERVARYSMEDAKVTYELGREFFPMEAQLSRLIGQSLWDVSRSSKGNLVEWFLLRKAYKRNELAPNKPDERELARRRGGYAGGYVKEPERGLWDNIVYMDFRSSAVSIIITHNVSPDTLNREGCKEYDVAPEVGHKFCKDFPGFIPSLLGDLLEERQKIKRKMKATVDPLEKKLLDYRQRVIKILANSFYGYMGYAKARWYCKECAESVTAWGREYIEMVIRELEEKFGFKVLYADTDGLHATIPGADAETVKKKAKEFLKYINPKLPGLLELEYEGFYVRGFFVTKKKYAVIDDEGKITTRGLEIVRRDWSEIAKETQARVLEAILKGGDVEEAVRIVKEVTEKLSKYEVPPEKLVIHEQITRDLRDYKATGPHVAVAKRLAARGVKIRPGTVISYIVLKGSGRIGDRAIPADEFDPTKHRYDAEYYIENQVLPAVERILKAFGYRKEDLRYQKTKQVGLGAWLKVKGKK

SEQ ID NO: 31 - protein, 775 aa, synthetic construct
MILDTDYITENGKPVIRVFKKENGEFKIEYDRTFEPYFYALLKDDSAIEDVKKVTAKRHGTVVKVKRAEKVQKKFLGRPIEVWKLYFNHPQDVPAIRDRIRAHPAVVDIYEYDIPFAKRYLIDKGLIPAEGDEELTMLAFAIATLYHEGEEFGTGPILMISYADGSEARVITWKKIDLPYVDVVSTEKEMIKRFLRVVREKDPDVLITYNGDNFDFAYLKKRSEELGIKFTLGRDGSEPKIQRMGDYFAVEVKGRIHFDLYPVIRRTINLPTYTLEAVYEAVFGKPKEKVYAEEIAQAWESGEGLERVARYSMEDAKVTYELGREFFPMEAQLSRLIGQSLWDVSRSSKGNLVEWFLLRKAYKRNELAPNKPDERELARRRGGYAGGYVKEPERGLWDNIVYLDIRSAAPSMIITHNVSPDTLNREGCKEYDVAPEVGHKFCKDFPGFIPSLLGDLLEERQKIKRKMKATVDPLEKKLLDYRQRVIKILANSFYGYGGYAKARWYCKECAESVAAWGREYAEMVIRELEEKFGFKVLYADTDGLHATIPGADAETVKKKAKEFLKYINPKLPGLLELEYEGFYVRGFFVTKKKYAVIDDEGKITTRGLEIVRRDWSEIAKETQARVLEAILKGGDVEEAVRIVKEVTEKLSKYEVPPEKLVIHEQITRDLRDYKATGPHVAVAKRLAARGVKIRPGTVISYIVLKGSGRIGDRAIPADEFDPTKHRYDAEYYIENQVLPAVERILKAFGYRKEDLRYQKTKQVGLGAWLKVKGKK

SEQ ID NO: 32 - protein, 775 aa, synthetic construct
MILDTDYITENGKPVIRVFKKENGEFKIEYDRTFEPYFYALLKDDSAIEDVKKVTAKRHGTVVKVKRAEKVQKKFLGRPIEVWKLYFNHPQDVPAIRDRIRAHPAVVDIYEYDIPFAKRYLIDKGLIPAEGDEELTMLAFAIATLYHEGEEFGTGPILMISYADGSEARVITWKKIDLPYVDVVSTEKEMIKRFLRVVREKDPDVLITYNGDNFDFAYLKKRSEELGIKFTLGRDGSEPKIQRMGDYFAVEVKGRIHFDLYPVIRRTINLPTYTLEAVYEAVFGKPKEKVYAEEIAQAWESGEGLERVARYSMEDAKVTYELGREFFPMEAQLSRLIGQSLWDVSRSSKGNLVEWFLLRKAYKRNELAPNKPDERELARRRGGYAGGYVKEPERGLWDNIVYLDLRSSAVSTIITHNVSPDTLNREGCKEYDVAPEVGHKFCKDFPGFIPSLLGDLLEERQKIKRKMKATVDPLEKKLLDYRQRVIKILANSFYGYGGYAKARWYCKECAESVAAWGREYAEMVIRELEEKFGFKVLYADTDGLHATIPGADAETVKKKAKEFLKYINPKLPGLLELEYEGFYVRGFFVTKKKYAVIDDEGKITTRGLEIVRRDWSEIAKETQARVLEAILKGGDVEEAVRIVKEVTEKLSKYEVPPEKLVIHEQITRDLRDYKATGPHVAVAKRLAARGVKIRPGTVISYIVLKGSGRIGDRAIPADEFDPTKHRYDAEYYIENQVLPAVERILKAFGYRKEDLRYQKTKQVGLGAWLKVKGKK